(19) United States
(12) Patent Application Publication
(10) Pub. No.: US 2024/0301413 A1
(43) Pub. Date: Sep. 12, 2024

(54) COMPOSITIONS AND METHODS USEFUL FOR THE TREATMENT OF BONE MARROW FAILURE DISEASES ASSOCIATED WITH RIBOSOMOPATHIES

(71) Applicant: THE CHILDREN'S HOSPITAL OF PHILADELPHIA, Philadelphia, PA (US)

(72) Inventor: Wei Tong, Wynnewood, PA (US)

(73) Assignee: THE CHILDREN'S HOSPITAL OF PHILADELPHIA, Philadelphia, PA (US)

(21) Appl. No.: 18/550,070

(22) PCT Filed: Mar. 11, 2022
(86) PCT No.: PCT/US2022/019989

§ 371 Date: Sep. 11, 2023

Related U.S. Application Data
(60) Provisional application No. 63/159,937, filed on Mar. 11, 2021.

(51) Int. Cl. C12N 15/113; A61P 7/00; A61P 7/06

(52) U.S. Cl. CPC C12N 15/113; A61P 7/00; A61P 7/06; C12N 2310/20; C12N 2310/531

(57) ABSTRACT

Compositions and methods for the treatment of ribosomopathies and regeneration of hematopoietic stems cells are provided.

### CROSS-REFERENCE TO RELATED APPLICATION

This application claims priority of U.S. Provisional application No. 63/159,937 filed Mar. 11, 2021, the entire contents being incorporated herein by reference as though set forth in full.

### GRANT SUPPORT STATEMENT

The application was made with Government support under grant numbers R01DK127738, 2R01HL095675, and R01HL133828 awarded by the National Institutes of Health. The US government has certain rights in the invention.

### INCORPORATION BY REFERENCE OF MATERIAL SUBMITTED IN ELECTRONIC FORM

Applicant hereby incorporates by reference the Sequence Listing material filed in electronic form herewith. This file is labelled “SEQLIST.txt” dated Mar. 11, 2022 and is 11,716 bytes in size.

### FIELD OF THE INVENTION

This invention relates the fields of bone marrow failure diseases associated with ribosomal dysfunction and methods and compositions for ameliorating symptoms thereof. More specifically, the invention provides nucleic acid-based therapeutics which down modulate ZNF622, thereby restoring hematopoietic regeneration of CD34+ stem cells in patients in need thereof.

### BACKGROUND OF THE INVENTION

Several publications and patent documents are cited throughout the specification in order to describe the state of the art to which this invention pertains. Each of these citations is incorporated herein by reference as though set forth in full.

At the steady-state, protein synthesis rate is low in adult hematopoietic stem cells (HSCs) as compared with committed progenitors or differentiated cells (Signer et al., 2014). Tightly-regulated protein synthesis rate is critical for HSC maintenance and function (Signer et al., 2014; Signer et al., 2016). HSC expansion under regenerative or stress conditions demands increased ribosome biogenesis and protein synthesis. Appropriate ribosome biogenesis and assembly ensures the translation efficiency and fidelity of proteins, which are important for normal development as well as prevention of cancer. Mutations in ribosomal proteins and gene products affecting ribosome biogenesis and protein synthesis are associated with human diseases marked by hematopoietic dysfunction (Sulima et al., 2017). However, how ribosome assembly is regulated in HSCs remains poorly understood, as is its contribution to hematopoietic diseases.

The biogenesis of the two ribosomal subunits 40S and 60S occurs largely in the nucleus; upon nuclear export, they separately undergo final stages of maturation in the cytoplasm that are regulated by distinct assembly factors and incorporation of the last few cytoplasmic ribosome proteins (de la Cruz et al., 2015). These assembly factors proofread and protect key functional sites on the ribosome and prevent premature joining of 60S and 40S subunits, to ensure regulated formation of functional 80S monosomes and appropriate translation (Klinge and Woolford, 2019).

“Ribosomopathies” are characterized by a group of inherited bone marrow failure (BMF) syndromes with impaired ribosome function. Individuals with “ribosomopathies” are deficient in HSCs or specific lineages of blood formation, and yet are predisposed to elevated leukemia and cancer risks (Ruggero and Shimamura, 2014). One such example is Shwachman-Diamond syndrome (SDS), that is etiologically linked to ribosome dysfunction arising from mutations of ribosome assembly factors (Warren, 2018; Woloszynek et al., 2004). Germline mutations in three different genes (SBDS, DNAJC21, and EFL1) involved in the 60S maturation and assembly all cause SDS (Boocock et al., 2003; Dhanraj et al., 2017; Tan et al., 2019; Tummala et al., 2016; Woloszynek et al., 2004), implying that HSCs are especially sensitive to perturbations in ribosome assembly. These mutations result in ribosomal subunit joining defects and decreased protein synthesis rate (Finch et al., 2011; Menne et al., 2007; Tan et al., 2019; Wong et al., 2011). The question of how ribosomal abnormalities cause marrow failure and cancer predisposition is therefore of fundamental biological and clinical interest. However, how assembly factors themselves are regulated and their impact on hematopoietic regeneration remain poorly understood.

### SUMMARY OF THE INVENTION

In accordance with the present invention, a method of lowering ZNF622 levels in a human stem cell (HSC) and improving HSC reconstitution ability of the cell is disclosed. An exemplary method entails administration of a therapeutically effective amount of a composition comprising a synthetic nucleic acid molecule targeting ZNF622 to a cell, wherein lowering ZNF622 levels in said cell increases ribosomal subunit joining thereby improving protein synthesis rates in the cell or tissue, wherein the synthetic nucleic acid molecule is selected from an shRNA, an siRNA, an antisense oligonucleotide, and a guide strand suitable for CRISPR editing ZNF622 targeted nucleic acids. Also provided is a method of treating, delaying the onset of, ameliorating, and/or reducing a disease, disorder and/or condition, or a symptom thereof, associated with one or more ribosomopathy in a patient in need thereof, comprising administering to cells in the patient a therapeutically effective amount of a synthetic nucleic acid targeting ZNF622, wherein the disease, disorder and/or condition, or the symptom thereof, associated with altered ribosomal subunit joining is treated, inhibited, the onset delayed, ameliorated, and/or reduced in the patient, wherein the synthetic nucleic acid molecule is selected from an shRNA, an siRNA, an antisense oligonucleotide, and a guide strand suitable for CRISPR editing. In certain embodiments, the synthetic nucleic acid targeting ZNF622 is modified to increase stability and/or uptake in vivo. In other embodiments, the synthetic nucleic acid targeting ZNF622 is an shRNA. In a preferred embodiment, the shRNA is cloned into a lentiviral vector using sequences shown in FIG. 15A. As noted, the synthetic nucleic acids may be delivered to the cell in a vector. Other suitable vectors include without limitation, a plasmid vector, a retroviral vector, an AAV vector, and an adenovirus associated vector. In other approaches, CRISPR editing of a ZNF622 encoding nucleic acid is employed using guide strands shown in FIGS. 15B and 15C.

In certain embodiments, the nucleic acid is modified and has a nucleobase sequence that is at least 90%, at least 95%, at least 99%, or 100% complementary to all or a portion of a human ZNF622 nucleic acid. The modified oligonucleotide can comprise at least one modified internucleoside linkage, at least one nucleoside of the modified oligonucleotide comprises a modified sugar or at least one nucleoside of the modified oligonucleotide comprises a modified nucleobase. Any of the methods described above can comprise administration of one or more additional agents useful for treatment of bone marrow failure.

The present invention also provides compositions for reducing expression of ZNF622, comprising a synthetic nucleic acid molecule targeting, and specifically hybridizing to, a ZNF622 encoding nucleic acid, selected from an shRNA, an siRNA, an antisense oligonucleotide, and a guide strand suitable for CRISPR editing in a biologically acceptable carrier. In certain aspects, the synthetic nucleic acid is modified to increase stability in bodily fluids and/or uptake in a cell of interest. In preferred embodiments, the synthetic nucleic acid targets all or a portion of the ZNF622 encoding nucleic acid shown in FIG. 15. The compositions of the invention can be formulated for ex vivo intracellular administration, parenteral administration, and intravenous administration. In particularly preferred embodiments, the composition is formulated for administration into autologous human stem cells, which are then reintroduced into the patient to be treated. Accordingly, methods for transforming human stem cells with the ZNF622 inhibitory nucleic acids of the invention and reintroduction of said transformed cells into a subject for the treatment of ribosomapathy also form an aspect of the invention. Stem cells to be transformed can be autologous or obtained from an immunologically compatible donor.

As discussed above, the compositions and methods disclosed herein can be used to advantage for the treatment of bone marrow failure. In certain embodiments, the bone marrow failure disorder is associated with anemia. The anemia may be hereditary anemia, myelodysplastic syndrome or severe chronic hemolysis. In other embodiments, the anemia is associated with cancer. Hereditary anemias include, for example, sickle cell anemia, thalassemia, Fanconi anemia, Diamond Blackfan anemia, Shwachman Diamond syndrome, and red cell membrane disorders.

### BRIEF DESCRIPTION OF THE DRAWINGS

FIGS. 1A-1E. Generation of hematopoietic-specific Hectd1 conditional knockout mice. Related to FIG. 3. (FIG. 1A) Strategy of generating Vav-cre mediated conditional Hectd1 knockout (Hectd1 f/f;Vav) mice. Exon3 was flanked by floxP sites. Deletion of exon 3 results in an early stop codon in exon4, producing a 50 aa (amino acid) truncated protein. (FIG. 1B) Genotyping of individual bone marrow-derived hematopoietic colonies from Hectd1 f/f and Hectd1 f/f;Vav mice to evaluate Hectd1 deletion efficiency. (FIG. 1C) RT-qPCR analysis to evaluate the deletion efficiency of Hectd1 at the mRNA level. Hectd1 contains 43 exons. qPCR primer pairs targeting to exon 3 are designed and used for qPCR analysis. BM cells from two mice of each genotype were tested. (FIG. 1D) As negative controls, qPCR primer pairs targeted to Hectd1 exon 6, 22, 38 were designed and used to confirm the specificity of Hectd1 excision in mice. (FIG. 1E) Western blot analysis of BM cells to confirm Hectd1 knockout efficiency at the protein level. Data are represented by mean±SD in (FIG. 1C, FIG. 1D).

FIGS. 2A-2Q. Hematopoietic-specific Hectd1 knockout mice show relatively normal steady-state hematopoiesis. Related to FIG. 3. (FIG. 2A) Complete blood count (CBC) analysis of peripheral blood (PB) from Hectd1 f/f (n=13) and Hectd1 f/f;Vav (n=13) mice. WBC, whole blood count; NE, neutrophils; LY, lymphocytes; MO, monocytes; PLT, platelets; RBC, red blood cells; HB, hemoglobin; HCT, hematocrit. (FIG. 2B) Lineage cell percentages in the PB of Hectd1f/f (n=8) and Hectd1 f/f;Vav (n=6) mice were analyzed by flow cytometry. (FIG. 2C) Total BM cellularity of Hectd1f/f (n=5) and Hectd1 f/f;Vav (n=6) mice. (FIG. 2D) Spleen weight of Hectd1f/f (n=5) and Hectd1 f/f;Vav (n=5) mice. (FIG. 2E, FIG. 2F) Flow cytometry analysis of the frequency of various HSPC subpopulations in BM (FIG. 2E) and spleen (FIG. 2F) of Hectd1f/f (n=5) and Hectd1 f/f;Vav (n=6) mice. Long-term hematopoietic stem cell (LT-HSC) is defined as CD150+CD48⁻Flk2⁻LSK cells. Short-term HSC (ST-HSC): CD150⁻CD48⁻Flk2⁻LSK. MPP2: CD150+CD48+Flk2⁻LSK. MPP3: CD150⁻CD48+Flk2⁻LSK. MPP4: CD150⁻Flk2+LSK. (FIG. 2G-FIG. 2H) Cell numbers of HSPC subpopulations in BM (FIG. 2G) and spleen (FIG. 2H) of Hectd1f/f (n=5) and Hectd1 f/f;Vav (n=5) mice. (FIG. 2I, FIG. 2J) Frequency (FIG. 2I) and cell number (FIG. 2J) of different hematopoietic progenitors in BM of Hectd1f/f (n=5) and Hectd1 f/f;Vav (n=6) mice as determined by flow cytometry are shown. (FIG. 2K) Colony-forming units (CFU)-E (left) and CFU-G/M (right) of the BM are shown. (FIG. 2L, FIG. 2M), Frequency (FIG. 2L) and cell number (FIG. 2M) of different hematopoietic progenitors in the spleen of Hectd1f/f (n=5) and Hectd1 f/f;Vav (n=6) mice are shown. (N) CFU-E (left) and CFU-G/M (right) analysis of the spleen are shown. (FIG. 2O-FIG. 2P) Cell cycle analysis of HSCs (FIG. 2O) and MPPs (FIG. 2P) in primary Hectd1f/f (n=5) and Hectd1 f/f;Vav (n=5) mice as determined by Ki67 and DAPI. (FIG. 2Q) Cell apoptosis analysis of LSKs, HSCs and MPPs in primary Hectd1f/f (n=4) and Hectd1 f/f;Vav (n=3) mice by Annexin V staining. All the experiments above were performed in 8-10 weeks old young mice. Each symbol represents an individual mouse; bars indicate mean values; error bars indicate SE. *: p<0.05; **: p<0.01; ***: p<0.001; ns: not significant, as determined by two-tailed Student's t-tests.

FIGS. 3A-3I. Hectd1-deficient BMs display a defective reconstituting ability and reduced functional HSC frequency. (FIG. 3A) Experimental scheme of serial BM transplantation assay. (FIG. 3B) Representative flow plots of donor/competitor/host chimerism in the peripheral blood (PB) of recipient mice after transplantation. (FIG. 3C) Donor chimerisms in the PB of recipient mice were measured every 4 weeks and the results are graphed. HectdJf (n=11) and Hectd1^{f/f;Vav} (n=11). (FIG. 3D) Lineage reconstitutions of donor-derived cells in primary recipients at 16 weeks post-transplantation are shown. Hectd1^{f/f} (n=11) and Hectd1^{f/f;Vav} (n=11). (FIG. 3E) Percentages of donor-derived HSPC subpopulation in the BM of primary transplanted mice at 16 weeks are shown. Hectd1^{f/f} (n=6) and Hectd1^{f/f;Vav} (n=6). (FIG. 3F) Donor percentages in the PB of secondary BMT recipients were analyzed every four weeks and the results are graphed. Hectd1^{f/f} (n=10) and Hectd1^{f/f;Vav} (n=11). (FIG. 3G) Experimental scheme of limiting dilution BMT to assess functional HSC frequency of Hectd1f/f and Hectd1^{f/f;Vav} BMs. (FIG. 3H) The results are presented as number of positively engrafted mice versus total number of mice analyzed for the indicated doses. Positive engraftment was defined as >1% donor-derived cells in the PB. CRU: competitive repopulating unit. 1SE: one standard deviation. (FIG. 3I) Donor chimerisms in the PB of recipient mice transplanted with different doses of BM cells at 16 weeks are shown. 100k: Hectd1f/f(n=5) and Hectd1^{f/f;Vav} (n=6); 30k Hectd1^{f/f} (n=10) and Hectd1^{f/f;Vav} (n=13); 10k: Hectd1^{f/f} (n=9) and Hectd1^{f/f;Vav} (n=7). In all relevant panels, each symbol represents an individual mouse; bars indicate mean frequencies; error bars indicate SE. *: p<0.05; **: p<0.01; ***: p<0.001; ns: not significant, as determined unpaired by two-tailed Student's t-test. See also FIGS. 1, 2 and 4A-4K.

FIGS. 4A-4M. Loss of Hectd1 does not impair hematopoietic differentiation, bone marrow niche or MPP reconstitution in transplanted mice. Panels 4A-4K are related to FIG. 3 and panels 4L-S4M are related to FIG. 5. (FIGS. 4A-4D) Loss of Hectd1 does not impair hematopoietic differentiation in transplanted mice. Examination of donor HSC differentiation in competitive total BM transplanted mice. 1*106 unfractionated BM cells from Hectd1f/f and Hectd1f/f;Vav mice were injected with the same number of competitor BM cells into lethally irradiated recipient mice. Donor chimerisms in different lineages of the PB were shown. (FIG. 4A) Granulocytes, (FIG. 4B) monocytes, (FIG. 4C) B cells and (FIG. 4D) T cells. Hectd1f/f (n=11) and Hectd1f/f;Vav (n=11). (FIGS. 4E-4G) Hectd1 deficiency leads to a decreased donor reconstitution in limiting dilution BMT. Donor cell percentage in recipients' PB injected with 100k (FIG. 4E), 30k (FIG. 4F), 10k (FIG. 4G) donor BM cells mixed with 300k competitor BM cells were examined every 4 weeks post-BMT. 100k: Hectd1f/f (n=5) and Hectd1f/f;Vav (n=6); 30k: Hectd1f/f (n=10) and Hectd1f/f;Vav (n=13); 10k: Hectd1f/f (n=9) and Hectd1f/f;Vav (n=7). (FIGS. 4H-4K) Hectd1 deficiency does not affect bone marrow niche as shown in reciprocal BMT. (FIG. 4H) Experimental scheme of reciprocal BMT 1×10⁶ total BM cells from SJL mice (CD45.1) were transplanted into lethally-irradiated Hectd1^{f/f} (n=8) and Hectd1 f/f;Vav (n=7) littermates (CD45.2). (FIG. 4I) Donor chimerism was analyzed by flow cytometry every 4 weeks post-BMT. (FIG. 4J) Donor-derived cells in each lineage 16 weeks post-BMT are shown. (FIG. 4K) Donor percentages in different HSPC subpopulations in the BM of Hectd1f/f and Hectd1 f/f;Vav recipient mice are shown. (FIGS. 4L-4M) HectD1 is required for HSC but not MPP/HPC reconstitution ability. (FIG. 4L) Percentages of donor HSC-derived myeloid cells (Gr1/Mac1+), B cells (B220+) and T cells (CD4+CD8+) are shown. (FIG. 4M) Percentages of donor MPP/HPC-derived myeloid cells (Gr1/Mac1+), B cells (B220+) and T cells (CD4+CD8+) are shown. In all relevant panels, each symbol represents an individual mouse; bars indicate mean frequencies; error bars indicate SE. *: p<0.05; **: p<0.01; ***: p<0.001; ns, not significant, as determined by two-tailed Student's t-tests.

FIGS. 5A-5L. HectD1 is required for HSC self-renewal in vivo and maintenance ex vivo. (FIG. 5A) Experimental scheme of HSC transplantation assay. LT-HSCs (LSK CD150+CD48⁻) from Hectd1^{f/f} and Hectd1^{f/f;Vav} mice were purified by flow cytometric sorting and 100 HSCs were either injected with 500K Scal-depleted competitor BMs into lethally irradiated recipient mice (Day0-BMT) or the resultant culture after 12 days was injected with 300K BMs into recipient mice (Day12-BMT). (FIG. 5B) Donor chimerisms in the PB of recipient mice transplanted with fresh HSCs (day0-BMT) were measured every 4 weeks and the results are shown in the graph. Hectd1^{f/f} (n=8) and Hectd1^{f/f;Vav} (n=8). (FIG. 5C) Percentages of donor-derived HSPC subpopulations in the BM of day0-BMT recipient mice 16 weeks post-transplant are shown. Hectd1^{f/f} (n=6) and Hectd1^{f/f;Vav} (n=5). (FIG. 5D) Donor chimerisms of day12 cultured HSC transplants (Day12-BMT) in the PB of recipient mice were measured every 4 weeks and the results are shown in the graph. Hectd1^{f/f} (n=7) and Hectd1^{f/f;Vav} (n=5). (Fig. SE) Percentages of donor-derived HSPC subpopulations in the BM of day12-BMT recipient mice 16 weeks post-transplant are shown. Hectd1^{f/f} (n=6) and Hectd1^{f/f;Vav} (n=4). (FIG. 5F) Representative images of ex vivo cultured HSCs at day 8. (FIGS. 5G-5I) Cell numbers of ex vivo cultured HSCs at different time points in different combinations of cytokines are shown. n=3 in each group. (FIG. 5J) Experimental scheme of HSC versus MPP/HPC transplantation assay. HSCs (LSK CD150⁺CD48⁻) or MPP/HPCs (LSK CD150⁻CD48⁺) were sorted from Hectd1f/f and Hectd1^{f/f;Vav} mice. 500 HSCs or 5000 MPP/HPCs were transplanted into each sub-lethally irradiated recipient mice. (FIGS. 5K-5L) Donor chimerisms in the PB of recipient mice were measured by flow cytometry every week post-BMT. Donor chimerisms of HSC (FIG. 5K) and MPP/HPC (FIG. 5L) transplants are shown. Hectd1^{f/f} (n=7-9) and Hectd1^{f/f;Vav} (n=6-7). Data in (Figs. SG-5I) are represented by mean±SD. In all relevant panels, each symbol represents an individual mouse; bars indicate mean frequencies; error bars indicate SE. *: p<0.05; **: p<0.01; ***: p<0.001, as determined by unpaired two-tailed Student's t-test. See also FIG. 4L-4M.

FIGS. 6A-6J. Hectd1 deficiency decreases RPS6 phosphorylation independent of the mTOR pathway. Related to FIG. 7. (FIGS. 6A-6B) Hectd1 deficiency decreases RPS6 phosphorylation in 2 day-cultured LSK cells. (FIG. 6A) A diagram showing different signaling pathways that we examined in LSKs and TF-1 cells. (FIG. 6B) Two-day cultured LSKs from Hectd1f/f and Hectd1 f/f;Vav mice were used to examine various signaling molecules in the mTOR pathway, peIF2a, and JAK/STAT, PI3K-AKT, MAPK pathways by WB. HECTD1 depletion in TF-1 cells recapitulates the signaling properties observed in Hectd1-deficient LSK cells. (FIGS. 6C-6D) Signal kinetics (FIG. 6C) and sensitivity to cytokines (FIG. 6D) of JAK-STAT, PI3K-AKT, RAS-ERK and mTOR pathways were compared in shLuc and shHECTD1 TF-1/MPL cells stimulated with hTPO. (FIG. 6E) Activation of the mTOR pathway was compared in shLuc vs shHECTD1 cells upon stimulation of a graded concentration of fetal calf serum (FCS). Phospho-RPS6 (pRPS6) are highlighted in red. (FIGS. 6F-6J) HectD1 does not affect RPS6 ubiquitination, S6K1-RPS6 interaction or the transcription of RiBi genes. (FIG. 6F) 293T cells were transfected with constructs expressing HA control or HA-tagged HectD1 WT or HectdD1 E3-dead mutant C2579G. 48 hrs later, cells were treated with or without proteasome inhibitor MG132 for 2 hr, followed by His-Ub pull down under denatured conditions. (FIG. 6G) 293T cells transfected with HA control or HA-tagged HectD1 WT or mutant C2579G were either untreated or deprived of serum. Activation of S6K1 and RPS6 by serum is shown in the WB (Pre-IP). S6K1-RPS6 interaction was assessed by S6K1 immunoprecipitation (IP) followed by WB with anti-RPS6 antibodies. (FIG. 6H) HectD1 depletion does not affect S6K1-RPS6 interaction as determined by IP/WB in TF-1 cells. (FIG. 6I) Total RNA was extracted and compared in HSCs and MPPs of Hectd1f/f and Hectd1 f/f;Vav mice. (FIG. 6J) qRT-PCR analysis of the gene expression of RiBi proteins and ribosome proteins in HSCs of Hectd1f/f and Hectd1 f/f;Vav mice. n=3 in each group.

FIGS. 7A-7M. HectD1 interacts with, ubiquitinates, and degrades ZNF622. (FIG. 7A) Freshly purified LSKs from Hectd1f/f and Hectd1^{f/f;Vav} mice were used to examine various signaling molecules by WB using the indicated antibodies. (FIG. 7B) TF-1/hMPL cells stably depleted of HECTD1 using two different shRNAs were generated along with shRNA to Luciferase (Luc). Cell lysates were subjected to WB analysis using indicated antibodies. (FIGS. 7C, 7D) TF-1/hMPL shLuc or shHECTD1 cells were cultured in triplicates in different concentrations of GM-CSF (FIG. 7C) or TPO (FIG. 7D). Cell growth after 3 days' culture were determined by MTT absorbance. (FIG. 7E) Silver staining gel image of a representative large-scale protein purification result to evaluate the efficiency and specificity of affinity purification of HA-HectD1 interacting proteins. * indicates the HA-HectD1 bait. IgG-H: indicates the Immunoglobin heavy chain. (FIG. 7F) CRAPome analysis of Hectd1-intearacting proteins from three independent IP-MS results revealed the SAINT probability over fold changes. ZNF622 was identified as an Hectd1 interactor and highlighted in red. (FIG. 7G) co-IP/WB analysis confirmed the interaction between Flag-ZNF622 and endogenous HectD1 in Flag-ZNF622 reconstituted TF-1 cells. (FIG. 7H) ZNF622 protein levels were increased in Hectd1^{f/f;Vav} LSKs compared to that of Hectd1^{f/f}/LSKs. (FIG. 7I) Quantification of ZNF622 protein levels from three independent experiments as in (FIG. 7H) is plotted. (FIG. 7J) ZNF622 mRNA levels were not affected in Hectd1-deficient LSKs as shown by qRT-PCR analysis. n=3 in each group. (FIG. 7K) TF-1 cells stably depleted of HECTD1 using two different shRNAs were treated with cycloheximide (CHX) for indicated times. ZNF622 half-lives were determined by WB. Representative blots of 3 independent experiments are shown. S. E., short exposure; L.E., long exposure. (FIG. 7L) Relative ZNF622 levels normalized to Luc time 0 (left panel) and that normalized to respective time 0 (right panel) as shown in (FIG. 7J). (FIG. 7M) 293T cells were transfected with HA-HectD1 or E3-dead mutant HectD1, along with Flag-ZNF622 and His-Ub or Ub mutant constructs as indicated. Cells were subjected to lysis in denatured condition followed by Ni²⁺beads-pulldown. Ubiquitinated proteins were detected by WB using indicated antibodies. In all relevant panels, data are represented by mean±SD. p-values are determined by unpaired two-tailed Students' t-test. *: p<0.05; **: p<0.01; ***: p<0.001. See also FIG. 6.

FIGS. 8A-81. Gene Ontology (GO) cellular component analysis of HectD1 interactors identified in three biological replicates of HA-HectD1 immunoprecipitation/mass spectrometry (IP/MS) Panels 8A-8F are related to FIG. 7. Panels 8G-8I, related to FIG. 9. (FIG. 8A). GO categories with fold enrichment>5 and FDR cutoff of 5% are shown. (FIGS. 8B-8E) Mapping the interacting domains/regions of HectD1 and ZNF622 by IP/WB. (FIG. 8B) Schematic demonstration of HectD1 WT, E3-dead mutant (C2579G) and deletion mutants (Fragment1-5). Positive interaction is labeled with “+”; no interaction “−”. (FIG. 8C) HA-IP/WB in293T cells transfected with HA-tagged HectD1 WT or indicated mutants. Endogenous ZNF622 was immunoblotted. The full-length HectD1 band was marked by asterisk. Fragment 2 (F2) encompassing amino acid 396-613 was lowly expressed and marked by a solid arrow. (FIG. 8D) Schematic demonstration of full-length ZNF622 and its deletion mutants. Positive interaction is labeled with “+”; no interaction “−”. (FIG. 8E) Flag-IP/WB in293T cells transfected with Flag-tagged ZNF622 FL or indicated truncates/mutants. Endogenous HectD1 was immunoblotted. (FIG. 8F) qRT-PCR analysis of the mRNA level of ZNF622 in TF-1 cells with shLuc or shHECTD1 #1 and #2. n=3 in each group. Data are represented by mean: SD. ns, not significant; ***: p<0.001, as determined by unpaired two-tailed Student's t-tests. (FIGS. 8G-81) Both protein synthesis rate and cell apoptosis are not changed in primary Hectd1 f/f;Vav mice.

FIG. 9A-9J. Hectd1 deficiency reduces HSC frequency and protein translational rate upon proliferative stress (FIGS. 9A-9D) Hectd1^{f/f} and Hectd1^{f/f;Vav} mice were injected with 150 mg/kg 5-FU, and euthanized at 10 days later for subsequent analysis. (FIG. 9A) HSC and MPP numbers in the BM of 5-FU challenged Hectd1^{f/f} (n=8) and Hectd1^{f/f;Vav} (n=8) mice are shown. (FIG. 9B) Representative histogram plot of protein synthesis rate in BM HSCs of 5-FU challenged mice as determined by in vivo OP-Puro assay. (FIG. 9C) Quantification of protein synthesis rate in HSCs and MPPs of 5-FU challenged Hectd1^{f/f} (n=6) and Hectd1^{f/f;Vav} (n=5) mice as shown in (FIG. 9B). (FIG. 9D) Percentages of BM HSCs and MPPs in the S phase of the cell cycle as determined by in vivo BrdU assay. Hectd1^{f/f} (n=3) and Hectd1^{f/f;Vav} (n=3). (FIG. 9E-9H) Hectd1^{f/f} and Hectd1^{f/f;Vav} mice were injected with cyclophosphamide (Cy) followed by two consecutive daily injections of G-CSF. Mice were euthanized one day after the last injection for subsequent analysis. (FIG. 9E) HSC and MPP numbers in the BM of Cy+2GCSF challenged Hectd1^{f/f} (n=7) and Hectd1^{f/f;Vav} (n=7) mice. Data are pooled from 4 independent experiments and unique symbols indicate mice from different experiments. (FIG. 9F) Representative histogram plot of protein synthesis rate in BM HSCs of Hecad1^{f/f} and Hectd1^{f/f;Vav} mice as determined by in vivo OP-Puro assay. (FIG. 9G) Quantification of protein synthesis rate in HSCs and MPPs of Hectd1^{f/f} (n=4) and Hectd1^{f/f;Vav} (n=4) mice as shown in (FIG. 9F). (FIG. 9H) Percentages of BM HSCs and MPPs in the S phase of the cell cycle as determined by in vivo BrdU assay. Hectd1^{f/f} (n=3) and Hectd1^{f/f;Vav} (n=4). (I) Protein synthesis rates of 2-day cultured LSKs from Hectd1^{f/f} and Hectd1^{f/f;Vav} mice were determined by OP-puro incorporation of newly synthesized protein after 1 hr labelling. Representative histogram plot is shown. (FIG. 9J) Quantification of relative protein synthesis rates of 2-day cultured LSKs from three independent experiments using OP-Puro assays as shown in (FIG. 9I). (FIG. 9K) Relative CFU-GM progenitors from Hectd1^{f/f} (n=3) and Hecad1^{f/f;Vav} (n=3) BMs in the presence of various concentrations of the translation elongation inhibitor puromycin is shown. Data in (9A, 9C, 9D, 9E, 9G and 9H) are represented by mean±SE. Data in (FIG. 9J, and FIG. 9K) are represented by mean±SD. p-value in (FIG. 9E) is determined by paired two-tailed Students' t-test; p-values in other panels are determined by unpaired two-tailed Students' t-test. *: p<0.05; **: p<0.01; ***: p<0.001; ns, not significant. See also FIG. 8.

FIGS. 10A-10J. Hectd1 deficiency results in an accumulation of ZNF622 and eIF6 in the 60S and a reduction in ribosomal subunit joining, which is restored by ZNF622 depletion. (FIG. 10A) Polysome profiling analysis of 2 day-cultured LSKs from Hectd1^{f/f} and Hectd1^{f/f;Vav} mice. (FIG. 10B) Quantifications of 60S:40S ratio (left panel) and 60S:80S ratio (right panel) from polysome profiling assay of TF-1 cells expressing shLuc or shHECTD1. Three independent experiments were performed. (FIG. 10C) Fractions from sucrose gradients (7%-45%) of TF-1 cell lysates stably expressing shLuc or shHECTD1 were collected and subjected to WB analysis. Representative results of three independent experiments are shown. Fractions 1-3 are cytoplasmic soluble proteins. 40S, 60S, 80S monosome and polysome fractions are indicated by colored lines, arrows, and fonts. Whole cell lysate (WCL). AF: assembly factor; RPL: ribosome protein large unit; RPS: ribosome protein small unit. WCL and sucrose fractions (shLuc and shHECTD1) were resolved in three SDS-PAGE gels in parallel. Sucrose fraction immunoblots were processed and developed in parallel, and images presented side-by-side. (FIG. 10D) Quantification of relative protein distribution in different polysome fractions as shown in (FIG. 10C). Relative protein levels in each fraction was normalized to the peak fraction of the indicated protein from the shLuc cells and plotted. n=3-4. (FIGS. 10E-10J) Knockdown of ZNF622 in HECTD1-deficient cells rescues ribosome composition, eIF6 release, as well as 60S/40S joining. (FIG. 10E) WB examination of knockdown efficiency in shLuc, HECTD1 single and HECTD1;ZNF622 double knockdown (DKD) cells. (FIG. 10F) Representative polysome profiles of TF-1 shLuc, HECTD1 and DKD cells. (FIG. 10G) Quantifications of 60S:40S ratio (left panel) and 60S:80S ratio (right panel) of polysome profiles as shown in (FIG. 10F). n=3. (H) Representative result of WB analysis with protein fractions from sucrose gradients (7%-45%) of TF-1 shLuc, HECTD1 and DKD cells (top panel). Quantification of eIF6 distribution in polysome fractions (bottom panel). N=3. (I) Ribosome dissociation/reassociation assay. Indicated TF-1 cell lines were lysed in 0.25 mM low Mg²⁺ buffer to dissociate ribosomal subunits (Top graph). MgCl₂ was subsequently added to a final concentration of 10 mM for ribosomal subunit reassociation (Bottom graph). Resultant cell lysates were loaded on a 7-45% sucrose gradient profiled. Representative graphs from three independent experiments are shown. (FIG. 10J) Quantification of 60S:40S ratios in the dissociated profiles (Top panel) and 80S:40S ratios in the reassociated profiles (Bottom panel). N=3. Note that the black line (shLuc) and the blue line (DKD) in (FIGS. 10F, 10H and 10I) superimpose. All data are represented by mean±SD. p-values are determined by unpaired two-tailed Students' t-test. *: p<0.05; **: p<0.01; ***: p<0.001; ns, not significant. See also FIG. 11A-11F.

FIG. 11. HECTD1 depletion reduces phospho-RPS6, but not NMD3 or select RPLs in ribosome fractions. Panels S6A-S6F are related to FIG. 10; 11G, related to FIG. 12. (FIGS. 11A-11F) Quantification of the relative distribution of indicated proteins in three independent polysome profiling assays of shLuc and shHECTD1 TF-1 cells. Data are represented by mean: SE. *: p<0.05; ns, not significant, as determined by unpaired two-tailed Student's t-tests. (FIG. 11G) Knockdown of ZNF622 does not change the phosphorylation of eIF2a or mTOR activation. WB of indicated proteins in TF-1 cells with single or double knockdown of HECTD1 and ZNF622.

FIG. 12. Knockdown of Znf622 in Hectd1-deficient cells restores protein synthesis rate and HSC reconstitution ability. (A) TF-1 cells stably expressing control shLuc, single or double knockdown of HECTD1 and ZNF622 were generated by lentiviral infection and sorting. WB analysis with indicated antibodies is shown. (B) Global protein synthesis rates of various TF-1 cells as in (A) were measured using OP-Puro assay. (C) Knockdown efficiency of 3 different shRNAs to mouse Znf622 in BaF3 cells is shown. shRNA #1 and #2 are chosen for subsequent BMT. (D) Schematic illustration of HSC lentiviral transduction/BMT strategy. (E) mCherry⁺ donor fractions in the PB were analyzed every 4 weeks post-BMT. Quantifications of mCherry⁺ % within donor from each group are shown. f/fVav+shLuc, n=5; f/f;Vav+shZnf622 #1, n=6; f/f;Vav+shZnf622 #2, n=5. (F) Quantifications of mCherry+donor % in the HSC and MPP fractions 16-weeks post BMT are shown. f/f;Vav+shLuc, n=5; f/f;Vav+shZnf622 #1, n=3; f/f;Vav+shZnf622 #2, n=4. (G) In a separate experiment, LSK cells were purified from Hectd1^{f/f} and Hectd1^{f/f;Vav} mice, infected with lentivirus expressing shLuc or shZnf622 #1, and subsequently transplanted. Quantifications of mCherry⁺ donor % in the PB from each group are shown. f/f+shLuc, n=8; f/f+shZnf622 #1, n=8; f/f;Vav+shLuc, n=7; f/f;Vav+shZnf622 #1, n=7. (H) Quantifications of mCherry⁺ donor percentages in the HSC and MPP fractions at the end of primary BMT are shown. f/f+shLuc, n=8; f/f+shZnf622 #1, n=8; f/f;Vav+shLuc, n=7; f/f;Vav+shZnf622 #1, n=7. (I) Two million BM cells from primary transplanted mice were harvested and transplanted into each secondary recipient. Quantifications of mCherry⁺ % within donor from each group in the secondary transplants are shown. f/f+shLuc, n=16; f/f+shZnf622 #1, n=13; f/f;Vav+shLuc, n=14; f/f;Vav+shZnf622 #1, n=9. In all relevant experiments, each symbol represents an individual mouse; horizontal lines indicate mean frequencies; error bars indicate SE. *: p<0.05; **: p<0.01; ***: p<0.001; ns, not significant, as determined by unpaired two-tailed Student's t-test. See also Figure S6G.

FIG. 13. Overlapping and distinct clinical features of inherited marrow failure associated with ribosomopathies. DBA, SDS, and DC are all characterized by marrow failure, predisposition to MDS/AML, and congenital abnormalities. The primary feature of marrow failure in DBA is red-cell aplasia, although other hematologc lineages may also be variably affected. Although neutropenia is the most common feature of marrow failure in SDS, all 3 lineages may be depressed. Cellular and humoral immunologic abnormalities have been reported in DC and SDS. The spectrum of physical anomalies in these three syndromes shares both overlapping and distinct features. Exocrine pancreatic lipomatosis is characteristic of SDS, whereas pulmonary fibrosis is a common characteristic of DC. The risk of soft tissue sarcoma is increased in DBA, and the risk of squamous cell carcinoma of the oropharynx and gastrointestinal tract is elevated in DC. Data are insufficient to determine whether solid tumor risk is elevated in SDS.

FIGS. 14A-14E: ZNF622 depletion restores protein synthesis and cell growth in SBDS-depleted hematopoietic cell lines. (FIG. 14A) SBDS-depleted TF-1 cells were generated which recapitulate the growth defects observed in human patients. The left panel shows the efficient knockdown of SBDS proteins using shRNA #2, and #2 by WB. The right panel shows that SBDS-depletion significantly reduces the growth of cytokine-dependent hematopoietic cell line, TF1 cells. (FIG. 14B-14C) To test if ZNF622 downregulation restores protein synthesis and cell growth of SBDS-depleted cells, an OP-Puro assay was performed to determine protein synthesis rates in shLuc, shZNF622, shSBDS or ZNF622; SBDS dual-depleted cells (left panels), and cell growth kinetics (right panels). The far right panel shows a representative OP-Puro flow cytometry plot. (FIG. 14B) shows the effect of ZNF622 knockdown on SBDS depleted cells transformed with shRNA-SBDS #2, while (FIG. 14C) shows the effect of ZNF622 knockdown on SBDS depleted cells transformed with shRNA-SBDS #3. (FIG. 14D) ZNF622 depletion restores clonogenic growth of primary CD34+ cells from SDS patients. Primary CD34⁺HSPCs (hematopoietic stem and progenitor cells) were isolated from BM aspirates of SDS patients and transduced with lentivirus expressing shLuc or two different shRNAs to ZNF622 along with the mCherry marker. Transduced cells were purified via flow cytometric sorting of mCherry positivity 2 days post-transduction, then plated onto semi-solid methylcellulose culture media. for CFU (colony forming unit) progenitors were enumerated 12-14 days later. Bars indicate mean values; error bars indicate SD. In all relevant panels, *: p<0.05; **: p<0.01; ***: p<0.001, as determined by two-tailed Student's t-tests. n=3 patient samples. (FIG. 14E) A schematic diagram illustrating how HectD1 controls ribosome assembly, protein translation, and HSC function via ZNF622. HectD1 insufficiency disrupts ribosome assembly and reduces protein synthesis rate, via an aberrant accumulation of ZNF622 in the 60S ribosome. This results in an SDS-like phenotype with a reduction in 60S/40S joining.

FIGS. 15A-15C. ShRNA (FIG. 15A) and CRISPR mediated (FIGS. 15B and 15C) modulation of ZNF622 expression. (FIG. 15A) Left: Western blot demonstrating the knockdown efficiency of ZNF622 by two different shRNAs in TF1 cells. Right: cDNA sequence of human ZNF622 gene with two shRNA target sequences highlighted in color. Bottom: nucleotide sequences of shRNA #1 and #2 to ZNF622. The 19-mer can be used as siRNA, while the 22-mer was used to generate shRNA. The oligo nucleotide sequences used to generate miR30-based shRNAs are also indicated. (FIG. 15B) Top: schematic representation of the domain structure of human ZNF622 protein. ZnF: Zinc finger domain; LR: Linker region. Right: Western blot demonstrating the knockout efficiency of ZNF622 by different gRNAs in TF1 cells. Middle: nucleotide sequences of gRNA 1, 2, and 3, which are located in exon 1. Bottom: Exon 1 nucleic acid sequence and protein sequence encoded by exon 1 of ZNF622 gene, with 3 gRNAs marked to top. (FIG. 15C) Top: nucleotide sequences of gRNA 4, 5, and 6, which are located in exon 2. Bottom: Exon 2 nucleic acid sequence and protein sequence encoded by exon 2 of ZNF622 gene, with 3 gRNAs marked to top.

### DETAILED DESCRIPTION OF THE INVENTION

Ribosomopathies compose a collection of disorders in which genetic abnormalities cause impaired ribosome biogenesis and function, resulting in specific clinical phenotypes. Congenital mutations in RPS19 and other genes encoding ribosomal proteins cause Diamond-Blackfan anemia, a disorder characterized by hypoplastic, macrocytic anemia. Mutations in other genes required for normal ribosome biogenesis have been implicated in other rare congenital syndromes, Schwachman-Diamond syndrome, dyskeratosis congenita, cartilage hair hypoplasia, and Treacher Collins syndrome. In addition, the 5q—syndrome, a subtype of myelodysplastic syndrome, is caused by a somatically acquired deletion of chromosome 5q, which leads to haploinsufficiency of the ribosomal protein RPS14 and an erythroid phenotype highly similar to Diamond-Blackfan anemia. Acquired abnormalities in ribosome function have been implicated more broadly in human malignancies.

HectD1, a member of HECT domain E3 ligases, plays an indispensable role in early embryogenesis. The HECT domain of HectD1 catalyzes the ubiquitination of its substrates to modulate protein stability, protein-protein interaction, and cellular localization. HectD1 has been reported to regulate various biological processes, including signaling transduction, gene transcription, development, and lipid homeostasis (Aleidi et al., 2018; Li et al., 2015; Sarkar and Zohn, 2012; Sugrue et al., 2019; Tran et al., 2013). To date, there have been no reports regarding the role of HectD1 in ribosomal functions or hematopoiesis. In the present invention, HectD1 has been identified as a critical determinant of HSC function via its direct ubiquitination of a ribosomal assembly factor. This discovery provides an important new therapeutic avenue to modulate ubiquitin-coordinated ribosomal assembly, thereby providing benefit to patients suffering from ribosomopathies.

### Definitions

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As employed above and throughout the disclosure, the following terms and abbreviations, unless otherwise indicated, shall be understood to have the following meanings.

In this invention, “a” or “an” means “at least one” or “one or more,” etc., unless clearly indicated otherwise by context. The term “or” means “and/of” unless stated otherwise. In the case of a multiple-dependent claim, however, use of the term “or” refers back to more than one preceding claim in the alternative only.

A “sample” refers to a sample from a subject that may be tested. The sample may comprise cells, and it may comprise body fluids, such as blood, serum, plasma, cerebral spinal fluid, urine, saliva, tears, pleural fluid, and the like. The sample may also be a tissue sample, or cells derived from a tissue.

The term “ribosomopathy” refers to a disease caused by abnormalities in the structure or function of ribosomal component proteins or rRNA genes, or other genes whose products are involved in ribosome biogenesis. “Hematopoiesis” refers to the highly orchestrated process of blood cell development and homeostasis. Prenatally, hematopoiesis occurs in the yolk sack, then liver, and eventually the bone marrow. In normal adults it occurs in bone marrow and lymphatic tissues.

The terms “stem cells” and “hematopoietic stem cells” are used interchangeably herein. Stem cells are distinguished from other cell types by two important characteristics. First, stem cells are unspecialized cells capable of renewing themselves through cell division, sometimes after long periods of inactivity. Second, under certain physiologic or experimental conditions, stem cells can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide under special conditions.

“Stem cells” can refer to multipotent stem cells that are capable of differentiating into all blood cells including erythrocytes, leukocytes and platelets. For instance, the “hematopoietic stem cells” or “stem cells” as used in the present invention are contained not only in bone marrow but also in umbilical cord blood derived cells.

As used herein, the term “hematopoietic compartment” refers to the cell compartment in a subject that contains all blood cell lineages, including without limitation, the myeloid lineage, which includes, without limitation, monocytes, macrophages, neutrophils, basophils, eosinophils, erythrocytes, megakaryocytes, platelets, and dendritic cells; and the lymphoid lineage, which includes, without limitation, T-cells, B-cells, NKT-cells, and NK cells. The “hematopoietic compartment” can contain all immature, mature, undifferentiated, and differentiated white blood cell populations and sub-populations, including tissue-specific and specialized varieties.

As used herein, the term “hematopoietic compartment cell formation” in a subject refers to the production and/or expansion of one or more cells of any blood cell lineages of the hematopoietic compartment in the hematopoietic compartment from hematopoietic stem cell (HSC) differentiation, HSC proliferation, and/or HSC survival. “Hematopoietic compartment cell formation” may be the result of HSC engraftment by exogenous HSCs, such as hematopoietic compartment reconstitution in an HSC transplant recipient. Alternatively, hematopoietic compartment cell formation” may be the result of endogenous HSC differentiation, endogenous HSC proliferation, and/or endogenous HSC survival, such as from hematopoietic compartment autoreconstitution in a subject.

A “patient,” “subject,” or “host” to be treated by the present methods refers to either a human or non-human animal, such as primates, mammals, and vertebrates. In particular, the terms refer to a human.

A “small molecule” refers to a composition that has a molecular weight of less than 3 about kilodaltons (kDa), less than about 1.5 kilodaltons, or less than about 1 kilodalton. Small molecules may be nucleic acids, peptides, polypeptides, peptidomimetics, carbohydrates, lipids or other organic (carbon-containing) or inorganic molecules. A “small organic molecule” is an organic compound (or organic compound complexed with an inorganic compound (e.g., metal)) that has a molecular weight of less than about 3 kilodaltons, less than about 1.5 kilodaltons, or less than about 1 kDa.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter, such as a chemical compound, a mixture of chemical compounds, a biological macromolecule, or an extract made from biological materials such as bacteria, plants, fungi, or animal (particularly mammalian) cells or tissues, which, when administered to a subject (human or animal) induces a desired pharmacological and/or physiologic effect by local and/or systemic action.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The terms “polynucleotide”, “nucleotide”, “nucleotide sequence”, “nucleic acid”, and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Upstream” and “Downstream” respectively refer to moving along a nucleotide strand in a 3′ to 5′ direction or a 5′ to 3′ direction.

“Introducing into” means uptake or absorption in the cell, as is understood by those skilled in the art. Absorption or uptake of oligos can occur through cellular processes, or via the use of auxiliary agents or devices.

The term “identity” as used herein and as known in the art, is the relationship between two or more oligo sequences, and is determined by comparing the sequences. Identity also means the degree of sequence relatedness between oligo sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While a number of methods to measure identity between two polynucleotide sequences are available, the term is well known to skilled artisans (see, e.g., Sequence Analysis in Molecular Biology, von Heinje, G., Academic Press (1987); and Sequence Analysis Primer, Gribskovm, M. and Devereux, J., eds., M. Stockton Press, New York (1991)). Methods commonly employed to determine identity between oligo sequences include, for example, those disclosed in Carillo, H., and Lipman, D., Siam J. Applied Math. (1988) 48:1073. In certain embodiments, the present invention may have 75, 80, 85, 90, 91, 92, 93, 94, 95, 96, 97, 98, 99, or 100% identity with the SEQ ID NOs disclosed herein.

“Substantially identical,” as used herein, means there is a very high degree of homology preferably >90% sequence identity.

The term “exogenous” nucleic acid can refer to a nucleic acid that is not normally or naturally found in or produced by a given bacterium, organism, or cell in nature. The term “endogenous” nucleic acid can refer to a nucleic acid that is normally found in or produced by a given bacterium, organism, or cell in nature.

The term “recombinant” is understood to mean that a particular nucleic acid (DNA or RNA) or protein is the product of various combinations of cloning, restriction, or ligation steps resulting in a construct having a structural coding or non-coding sequence distinguishable from endogenous nucleic acids found in natural systems.

As used herein the phrase “target transcript” refers to an RNA molecule encoding a target protein of interest.

As used herein, “target protein” means a protein that one desires to decrease in amount, concentration, or activity, thereby effecting a beneficial therapeutic change. In certain embodiments, the target protein is encoded by the primary open reading frame of a target transcript.

As used herein, “primary open reading frame” or “pORF” means the portion of the target transcript that encodes the primary protein associated with the transcript. In certain embodiments, the pORF encodes the target protein.

As used herein, “target site” means the portion of the target transcript having a nucleobase sequence that is complementary to a portion of the nucleobase sequence of a modified oligonucleotide. In certain embodiments, the modified oligonucleotide is complementary to the target site across the entire length of the modified oligonucleotide.

As used herein, “start site” means a group of nucleobases on a transcript at which a ribosomal subunit is recruited. In certain embodiments, a start site may result in initiation of translation. In certain embodiments, a start site is an AUG codon. In certain embodiments, a start site is a non-canonical start codon.

The term “RNA interference (RNAi) molecule” refers to any molecule inhibiting RNA expression or translation via the RNA reducing silencing complex (RISC) in a cell's cytoplasm, where the RNAi molecule interact with the catalytic RISC component argonaute. A small interfering RNA (siRNA) is typically a double-stranded RNA complex comprising a passenger (sense) and a guide (antisense) oligonucleotide (strand), which when administered to a cell, results in the incorporation of the guide (antisense) strand into the RISC complex (siRISC) resulting in the RISC associated inhibition of translation or degradation of complementary RNA target nucleic acids in the cell. The sense strand is also referred to as the passenger strand, and the antisense strand as the guide strand. A small hairpin RNA (shRNA) is a single nucleic acid molecule which forms a stem loop (hairpin) structure that is able to degrade mRNA via RISC.

RNAi nucleic acid molecules may be synthesized chemically (typical for siRNA complexes) or by in vitro transcription, or expressed from a vector. shRNA molecules are generally between 40 and 70 nucleotides in length, such as between 45 and 65 nucleotides in length, such as 50 and 60 nucleotides in length, and interacts with the endonuclease known as Dicer which is believed to processes dsRNA into 19-23 base pair short interfering RNAs with characteristic two base 3′ overhangs which are then incorporated into an RNA-induced silencing complex (RISC).

Typically, the guide (antisense) strand of an siRNA (or antisense region of a shRNA) is 17-25 nucleotide in length, such as 19-23 nucleotides in length and complementary to the target nucleic acid or target sequence. In an siRNA complex, the guide (antisense) strand and passenger (sense) strand form a double stranded duplex, which may comprise 3′ terminal overhangs of e.g. 1-3 nucleotides (resembles the product produced by Dicer), or may be blunt ended (no overhang at one or both ends of the duplex).

It will be recognized that RNAi may be mediated by longer dsRNA substrates which are processed into siRNAs within the cell (a process which is thought to involve the dsRNA endonuclease DICER). Effective extended forms of Dicer substrates have been described in U.S. Pat. Nos. 8,349,809 and 8,513,207, hereby incorporated by reference.

RNAi oligonucleotides may be chemically modified using modified internucleotide linkages and high affinity nucleosides such as 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA. See for example WO 2002/044321 which discloses 2′O-Methyl modified siRNAs, WO2004083430 which discloses the use of LNA nucleosides in siRNA complexes, known as siLNAs, and WO2007107162 which discloses the use of discontinuous passenger strands in siRNA such as siLNA complexes. WO03006477 discloses siRNA and shRNA (also referred to as stRNA) oligonucleotide mediators of RNAi. Harborth et al., Antisense Nucleic Acid Drug Dev. 2003 April; 13(2):83-105 refers to the sequence, chemical, and structural variation of small interfering RNAs and short hairpin RNAs and the effect on mammalian gene silencing.

In some embodiments RNAi oligonucleotides comprise one or more phosphorothioate internucleoside linkages. In RNAi molecules phosphorothioate internucleoside linkages may reduce or the nuclease cleavage in RICS it is therefore advantageous that not al internucleoside linkages are modified. Phosphorothioate internucleoside linkages can advantageously be place in the 3′ and/or 5′ end of the RNAi nucleic acid molecule, in particular in the of the part of the molecule that is not complementary to the target nucleic acid (e.g. the sense strand or passenger strand in an siRNA molecule). The region of the RNAi molecule that is complementary to the target nucleic acid (e.g. the antisense or guide strand in a siRNA molecule) may however also be modified in the first 2 to 3 internucleoside linkages in the 3′ and/or 5′ terminal.

The phrase “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as the flank (also known as wing) and gap region of a gapmer, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid.

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

The phrase “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprises a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

The phrase “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage, this is a particular advantage for therapeutic oligonucleotides. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In other approaches, the oligonucleotide, e.g. the therapeutic antisense oligonucleotide, shRNA or siRNA, comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate. The use of fully phosphorothioate modified oligonucleotides or contiguous nucleotide sequences is often used in antisense oligonucleotides, although in siRNAs partial phosphorothioate modifications may be preferred as fully phosphorothioate modifications have been reported to limit RNAi activity, particularly when used in the guide (antisense) strand. Phosphorothioate modifications may be incorporated into the 5′ and 3′ ends of an antisense strand of a siRNA without unduly limiting RNAi activity.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F, which the internucleoside linkage in region G may be fully phosphorothioate. Advantageously, all the internucleoside linkages in the contiguous nucleotide sequence of the antisense oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleosides, which according to EP2 742 135 may for example be tolerated in an otherwise DNA phosphorothioate the gap region.

The term “nucleobase” includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1. In some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

The phrase “modified oligonucleotide” describes an oligonucleotide comprising one or more sugar-modified nucleosides and/or modified internucleoside linkages. The term chimeric” oligonucleotide is a term that has been used in the literature to describe oligonucleotides with modified nucleosides.

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. nucleotides in length.

In certain approaches, a CRISPR system may be utilized to introduce nucleic acid changes that reduce expression of ZNF622. CRISPR is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

According to the present invention, the target nucleic acid is a nucleic acid which encodes mammalian ZNF622 and may for example be a gene, a RNA, a mRNA, and pre-mRNA, a mature mRNA or a cDNA sequence. The target may therefore be referred to as an ZNF622 target nucleic acid. The oligonucleotide of the invention may for example target exon regions of a mammalian ZNF622 RNA, or may for example target intron region in the ZNF622 pre-mRNA. An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting Hg-RPS23 mRNA may be between 15-35 nucleotides in length, and more typically about 21 nucleotides in length.

“Antisense oligos or strands” are oligos that are complementary to sense oligos, pre-mRNA, RNA or sense strands of particular genes and which bind to such genes and gene products by means of base pairing. When binding to a sense oligo, the antisense oligo need not base pair with every nucleoside in the sense oligo. All that is necessary is that there be sufficient binding to provide for a Tm of greater than or equal to 40° C. under physiologic salt conditions at sub-micromolar oligo concentrations.

“Antisense oligonucleotides” are single stranded oligonucleotides or “oligos” that inhibit the expression of the targeted gene by interfering with some step in the sequence of events leading to gene expression subsequent protein production by directly interfering with the step. Other oligo act by inducing gene target transcript digestion.

“Native RNA” is naturally occurring RNA (i.e., RNA with normal C, G, U and A bases, ribose sugar and phosphodiester linkages).

As used herein, “antisense activity” or “silencing activity” means any detectable and/or measurable inhibition of expression or function of the target attributable to the hybridization of an antisense compound to its target nucleic acid.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measurable activity” means a measurable activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenylation, addition of 5′-cap), translation, and post-translational modification.

As used herein, “translation” means the process in which a polypeptide (e.g. a protein) is translated from an mRNA. In certain embodiments, an increase in translation means an increase in the number of polypeptide (e.g. a protein) molecules that are made per copy of mRNA that encodes said polypeptide.

As used herein, “targeting” or “targeted to” means the association of an antisense compound or silencing compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

The terms “construct”, “cassette”, “expression cassette”, “plasmid”, “vector”, or “expression vector” is understood to mean a recombinant nucleic acid, generally recombinant DNA, which has been generated for the purpose of the expression or propagation of a nucleotide sequence(s) of interest, or is to be used in the construction of other recombinant nucleotide sequences.

The term “promoter” or “promoter polynucleotide” is understood to mean a regulatory sequence/element or control sequence/element that is capable of binding/recruiting an RNA polymerase and initiating transcription of sequence downstream or in a 3′ direction from the promoter. A promoter can be, for example, constitutively active, or always on, or inducible in which the promoter is active or inactive in the presence of an external stimulus. Example of promoters include T7 promoters or U6 promoters.

The term “operably linked” can mean the positioning of components in a relationship which permits them to function in their intended manner. For example, a promoter can be linked to a polynucleotide sequence to induce transcription of the polynucleotide sequence.

The terms “complementarity” or “complement” refer to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

As noted above, a nucleic acid as described herein can be “modified” to increase stability in vivo. Such modifications include, without limitation, sugar modifications such as 2′fluoro, 2′-O-methyl, 2′-NH₂. The phosphodiester backbone linkage can also be substituted with phosphorothioate as disclosed herein, but other backbone modifications such as triazole linked, or phNA are known to the skilled artisan. Additionally, modified bases can be employed, including without limitation, 7-deaza-dA, and carboxamide-dU.

In some aspects, the invention provides methods comprising delivering one or more polynucleotides, such as or one or more vectors as described herein (e.g., encoding all or portions of the base editing complexes discussed below), one or more transcripts thereof, and/or one or proteins transcribed therefrom, to a host cell. In some aspects, the invention further provides cells produced by such methods, and organisms (such as animals, plants, or fungi) comprising or produced from such cells. Conventional viral and non-viral based gene transfer methods can be used to introduce nucleic acids in mammalian cells or target tissues. Such methods can be used to administer nucleic acids encoding components of a base editing system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. In particularly preferred embodiments, the cell is a human stem cell.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be re-introduced into the human or non-human animal.

The following materials and methods are provided to illustrate certain embodiments of the invention. They are not intended to limit the invention in any way.

### Mouse Models

Hectd1 “knockout-first, conditional-ready” mouse line (C57B6/NGpt-Hectd1^{tm1a}/Gpt, #T000502) was obtained from EuComm (the ES lines were generated by European Consortium) and Model Animal Resource Information Platform of Nanjing University, China. FLPI recombinase transgenic mice (B6;SJL-Tg(ACTFLPe)9205Dym/J, #003800) and SJL (CD45.1) recipient mice were purchased from the Jackson Laboratory (Rodriguez et al., 2000). Vav1-Cre mice were originally generated by Dr. Thomas Graf (Stadtfeld and Graf, 2005) and generously provided by Dr. Nancy Speck (Chen et al., 2009). Hectd1 transgenic mice were first crossed with Rosa-Flippase mice to eliminate FRT-flanked LacZ and Neo mini-gene, and then with wild type mice to get rid of the Rosa-Flippase gene to minimize the possible effects of these elements in hematopoiesis. The resultant mice with loxP-flanked Hectd1 alleles (Hectd1^{f/f}) targeting exon 3 were crossed with Vav-cre transgenic mice to obtain the control Hectd1^{f/f} and Hectd1^{f/f;Vav} conditional knockout mice (cKO). All mice were bred and grown in house in pathogen-free animal facilities. Both male and female mice (8˜12 weeks old) were used and randomly assigned for all experiments. All the animal studies were performed under an approved protocol by the Institutional Animal Care and Use committee of the Children's Hospital of Philadelphia.

For 5-FU challenge, sex and age-matched (8˜12 weeks old) adult Hectd1^{f/f} and Hectd1^{f/f;Vav} mice were injected intraperitoneally with 5-Fluorouracil (5-FU, Cat #F6627, Sigma, 150 mg/kg body weight, pH8.5 in PBS) (Rozenova et al., 2015). Total BM cell count, HSC frequency, cell cycle and apoptosis assay were determined after 10 days of 5-FU administration. For Cy/GCSF induced stress, we intraperitoneally injected mice with 1 dose of cyclophosphamide (4 mg/mouse, Baxer) followed by two daily subcutaneous injections of 5 ug GCSF (Neupogen, Amgen). 1 day after the last GCSF injection, mice were euthanized for subsequent analysis (Morrison et al., 1997; Signer et al., 2014).

### Cell Lines

TF-1 cell lines were purchased from American Type Culture Collection (ATCC) and grown in RPMI-1640 media supplemented with 10% bovine calf serum (Cat #SH30072.03, HyClone), 2 mM L-glutamine (Cat #25030-081, Gibco) and penicillin/Streptomycin (Gibco) and 2 ng/mL GM-CSF (PeproTech) at 37° C. and 95% humidity in an atmosphere of 5% CO₂. 293T cells were from ATCC and grown in DMEM media supplemented with 10% bovine calf serum, 2 mM L-glutamine and penicillin/Streptomycin at 37° C. and 95% humidity in an atmosphere of 5% CO₂. BaF3 cells were maintained in RPMI-1640 media supplemented with 10% bovine calf serum and 10% WEHI supernatant at 37° C. and 95% humidity in an atmosphere of 5% CO₂.

Genotyping and qPCR

Mouse tail genomic DNAs were isolated with the standard proteinase K lysis protocol. Genotyping was performed by PCR. To evaluated Vav-cre excision efficiency in hematopoietic stem and progenitor cells, we plated total BM cells into M3434 methylcellulose semisolid media (StemCell Technologies Inc) for colony formation. Single colonies were picked, resuspended in 90 uL buffer (50 mM NaOH, 0.2 mM EDTA), and boiled for 20 min, followed by mixing with 10 uL 1M Tris, pH8.0 for neutralization. The lysates were subsequently used for genotyping. .

For quantitative real time PCR assay, total RNA was extracted using RNeasy Mini Kit (Qiagen). Reverse-transcription reaction was performed with random primers using qScript cDNA Supermix (Quanta Biosciences), and quantitative PCR was done with SYBR Green Master Mix (Applied Biosystems). Sequences of genotyping and qPCR primers are listed in Table 1.

TABLE 1
Genotyping and qPCR primer sequences
(F: forward; R: Reverse)

| | |
|---|---|
| Genotyping Primers | |
| Hectd1 FRTt F | TGCCTGCTATCAAGCCTGAT |
| Common_En2 R | CCAACTGACCTTGGGCAAGAACAT |
| Hectd1 FRTt R | CAGAGAGCTGGCAGGAAAGT |
| Hectd1 ES loxp F | TCCAGCTTGGTATTTGCCCA |
| Hectd1 ES loxp R | ACTGATGGCGAGCTCAGACC |
| Cre F | GCGGTCTGGCAGTAAAAACTATC |
| Cre R | GTGAAACAGCATTGCTGTCACTT |
| Internal control F | CTAGGCCACAGAATTGAAAGATCT |
| Internal control R | GTAGGTGGAAATTCTAGCATCATCC |
| qPCR Primers | |
| (F: Forward; | |
| R: Reverse) | |
| Human HECTD1 F | CCAGTGAGAGTGACACAGGT |
| Human HECTD1 R | ATCATCATCGAGCTCAGCCA |
| Human ZNF622 F | GGCTCACATGACCAAAGACC |
| Human ZNF622 R | CGTTGCACCACAAGCAAATC |
| Human GAPDH F | CCACCCATGGCAAATTCC |
| Human GAPDH R | TGGGATTTCCATTGATGACAAG |
| Mouse Hectd1-exon3 | AGCCCGTGCCATAACATACT |
| pair1 F | |
| Mouse Hectd1-exon3 | TCTCTGCTGGTCCTGTTGTT |
| pair1 R | |
| Mouse Hectd1-exon3 | ACTTTCCTGCCAGCTCTCTG |
| pair2 F | |
| Mouse Hectd1-exon3 | CTATCCTCCGGGTGCATTCT |
| pair2 R | |
| Mouse Hectd1-exon6 F | CTGGTTGACCTCCTCTTGGT |
| Mouse Hectd1-exon6 R | TCTATAAGCTGCCGATGGGA |
| Mouse Hectd1-exon22 F | GCCGCTTGGAGGACATACTA |
| Mouse Hectd1-exon22 R | AGACCTTCCATAGCCACGAG |
| Mouse Hectd1-exon38 F | ATGTACAGCGGTCATGTGGA |
| Mouse Hectd1-exon38 R | GTCGCCCATGCACATAAGTT |
| Mouse ZNF622 F | AGAGGAGGAGGGAGAGGAAG |
| Mouse ZNF622 R | CACTCCAGGGTCCTCACATT |
| Mouse Rps6 F | AGCTCCGCACCTTCTATGAGA |
| Mouse Rps6 R | GGGAAAACCTTGCTTGTCATTC |
| Mouse Rps7 F | AGCGCCAAGATCGTGAAGC |
| Mouse Rps7 R | CACCACCAACTTCGATTTCCTT |
| Mouse Rpl5 F | GGAGGTGAATGGAGGTGAATAC |
| Mouse Rpl5 R | AGTTGTAGTTCGGGCAAGAC |
| Mouse Rpl11 F | ATGGCGCAAGATCAAGGGG |
| Mouse Rpl11 R | GACTGTGCAGTGAACAGCAAT |

The sequence of qPCR primers for mouse NOP56, PWP2, GAR1, NOP14, Rrp9, Rrp12, Ddx18, Ub-C is from Chauvin et al., 2014 (SEQ ID NOS: 1 to 35 are shown in descending order)

### Constructs and Virus Packaging

pCMV-HA-HectD1 WT or C2579G mutant constructs were kindly gifted by Dr. Irene E. Zohn (Children's Research Institute, United States, Washington, DC, United States) (Sarkar and Zohn, 2012). The truncation mutants were amplified by PCR and subcloned into pCMV-HA vector. Human ZNF622 cDNA was amplified by PCR from a homemade cDNA library from TF-1 cells and constructed into a retroviral pOZ-FH vector that contains a Flag and a HA tag. All the deletion or truncation mutants were subcloned into the pOZ-FH vector. HectD1 or ZNF622 miR30-based shRNA constructs were subcloned into Lentiviral vector (pCL20.MSCV.mir30.PGK.mCherry) generously provided by Dr. Shannon McKinney-Freeman

### Complete Blood Count (CBC), Flow Cytometry of HSPCs and Lineage Cells

Peripheral blood was collected from 8˜12 week-old Hectd1^{f/f} and Hectd1^{f/f;Vav} mice into EDTA-coated tubes. CBC analysis was performed with a Hemavet 950 (Drew Scientific, Inc). For lineage staining, cells from peripheral blood were lysed with RBC lysis buffer (0.8% NH₄Cl, 10 uM EDTA, pH 7.4-7.6) for 10 min at 4° C. to remove red blood cells, followed by staining with different fluorochrome-conjugated anti-Gr-1 (RB6-8C5) (granulocytes), -Mac1 (M1/70) (macrophages), -B220 (RA3-6B2) (mature B cells), -CD4 (GK1.5) and -CD8 (53-6.7) (T cells) antibodies, for 30 min at 4° C. After washing with flow buffer (PBS containing 0.5% BSA), cells were suspended in flow buffer containing lug/mL propidium iodide (PI) or 2.5 ug/mL DAPI for flow cytometry analysis.

HSPCs staining was conducted as described previously (Lv et al., 2017). Cells from BM (2 femurs+2 tibias+2 hips for one mouse) or spleen were harvested in PBS containing 0.5% BSA, and quickly lysed with RBC lysis buffer for 1 min at 4° C. Cells were then stained with biotin-conjugated anti-Gr-1 (RB6-8C5), -Mac1 (M1/70), -B220 (RA3-6B2), -CD19 (eBio1D3), -Terl19 (TER-119), -CD5 (53-7.3), -CD4 (GK1.5), -CD8 (53-6.7), in combination with APC-Cy7-c-Kit (2B8), PerCP-Cy5.5-Scal (E13-161.7 or D7), FITC-CD48 (HM48-1), PE-Cy7-CD150 (TC15-12F12.2), APC-CD34 (RAM34), and PE-Flk2 (A2F10.1) antibodies for 30 min on ice, followed by secondary staining with streptavidin-PE-TexasRed (Invitrogen SA1017, 1:50) for 30 min on ice. Different HSPC subpopulations were defined as long-term stem cells (LT-HSCs, Lin⁻Sca1⁺c-Kit⁺Flk2⁻CD150⁺CD48⁻), short-term stem cells (ST-HSCs, Flk2⁻CD150⁻CD48⁻LSK), multiple potent progenitors (megakaryocyte/erythroid-biased MPP2, Flk2⁻CD150⁺CD48⁺LSK; myeloid-biased MPP3, Flk2⁻CD150⁻CD48⁺rLSK; lymphoid-biased MPP4, Flk2⁺CD150⁻LSK) (Pietras et al., 2015).

For committed progenitor cell staining, namely granulocyte-monocyte progenitor (GMP, CD34⁺CD16/32⁺Lin⁻c-Kit⁺Sca1⁻) cells, common myeloid progenitor (CMP, CD34⁺CD16/32⁻LKS⁻) cells and megakaryocyte-erythrocyte progenitor (MEP, CD34⁺CD16/32⁻LKS⁻), cells from the BM or spleen were stained with PE-FrRIII/II (CD16/CD32) for 30 minutes on ice after a quick RBC lysis, followed by blocking with rat serum, then stained with biotin-conjugated lineage panel as described above, along with APC-Cy7-c-Kit (2B8), PerCP-cy5.5-Sca1 (E13-161.7 or D7), APC-CD34 for 1h on ice.

Lineage cell FACS samples were analyzed on a BD FACS Canto flow cytometer, while HSPC and progenitor samples were analyzed on a BD FACS Fortessa flow cytometer. Data were analyzed on FlowJo (FlowJo, LLC).

### Competitive BMT and Limiting Dilution BMT

For competitive BMT, 1 million total BM cells from 8˜12-week-old Hectd1f/for Hectd1^{f/f;Vav} (CD45.2) mice were mixed with the same number of competitor total BM cells (CD45.1), and transplanted into lethally irradiated (a split dose of 10 Gy) recipient mice (CD45.1/2) by retro-orbital injection. Every four weeks after BMT, donor cell reconstitution in periphery blood (PB) was evaluated by flow cytometry. 16 weeks after BMT, reconstituted donor stem and progenitor cells (HSPCs) from BM or spleen were analyzed by flow cytometry.

For limiting dilution BMT, an increasing number (10k, 30k, 100k) of total BM cells from Hectd1^{f/f} or Hectd1^{f/f;Vav} (CD45.2) mice were mixed with a fixed number (300k) of competitor BM cells and transplanted into lethally irradiated recipient mice (Bersenev et al., 2008). 16 weeks after BMT, donor cell percentage in the PB was evaluated by flow cytometry. Mice with more than 1% of donor-derived cells were defined as “positive”. Data were analyzed by ELDA (Hu and Smyth, 2009).

### HSC Sorting and Transplantation

HSC purification and BMT were performed as described previously (Balcerek et al., 2018). Lineage positive cells were first depleted using a lineage cell depletion kit (Cat #130-090-858, Miltenyi Biotec). Lineage negative (Lin−) cells were then stained with APC-Cy7-c-Kit (2B8), PerCP-Cy5.5-Sca1 (E13-161.7 or D7), FITC-CD48 (HM48-1), PE-Cy7⁻CD150 (TC15-12F12.2). LT-HSCs were purified with MoFlo Astrios Sorter and 100 LT-HSCs were seeded in a round-bottom 96-well plate. These LT-HSCs were either transplanted on the day (DO BMT) or cultured in SFEM media supplemented with 100 ng/mL SCF and 20 ng/mL TPO for 12 days, and all the resultant cells were then transplanted (Day12 BMT). Specifically, 100 LT-HSCs (DO BMT) or 100 LT-HSCs-derived cells (CD45.2) at day12 (D12 BMT) were mixed with 500k Seal-depleted competitor BM cells (CD45.1 or CD45.1/2) and injected retro-orbically into lethally irradiated (10Gy) recipient mice (CD45.1/2 or CD45.1). Every four weeks after BMT, donor cell reconstitution in the PB was evaluated by flow cytometry. 16 weeks after BMT, reconstituted donor stem and progenitor cells (HSPCs) from BM or spleen were analyzed by flow cytometry.

### OP-Puro (O-Propargyl-Puromycin) Click-iT Protein Synthesis Assay

To detect protein synthesis rate in vivo, primary or stressed mice were injected intraperitoneally with OP-Puro (Cat #HY-15680, MCE; 50 mg/kg body weight, pH6.4-6.6 in PBS) for 1 hour before euthanasia (Signer et al., 2014). Total BM cells were harvested and live stained with cell surface markers for HSCs/MPPs after a quick RBC lysis. Cells were then fixed with BD Cytofix solution for 20 min on ice. After washing with BD Perm/Wash buffer, cells were permeabilized with BD Cytoperm Plus solution for 10 min on ice, followed by refixing in Cytofix solution for 5 min. The azide-alkyne reaction was performed using Click-iT plus OPP Alexa Fluor 647 or 488 kit (Cat #C10458, Invitrogen) for 30 min at room temperature. Cells were then washed and resuspended in flow buffer, and analyzed by on a BD FACS Fortessa flow cytometer.

For ex vivo analysis, OP-Puro was added to cell culture at the final concentration of 20 uM for 1 hour at a 37° C. incubator. The azide-alkyne reaction was performed as described above.

### Cell Cycle and Cell Apoptosis Assay

For BrdU cell cycle analysis, mice were injected with 200 uL BrdU (10 mg/mL, Cat #550891, BD Pharmingen) for 2 hours. Total BM cells were stained with cell surface markers for HSCs/MPPs, and then fixed and permeabilized with BD Cytofix/Cytoperm kit, followed by treatment with 300 ug/mL DNaseI for 1 hours at a 37° C. water bath. After washing with BD Perm/Wash buffer, cells were stained with FITC-anti-BrdU (Cat #5133284, BD Pharmingen) for 20 min at room temperature. After washing, cells were resuspended in flow buffer with DAPI (5 ug/mL), and analyzed by flow cytometry.

For apoptosis assay, total BM cells were stained with cell surface markers for HSCs/MPPs. After washing with flow buffer, cells were resuspended in 200 uL Annexin V binding buffer. 10 uL FITC-Annexin V (Cat #55647, BD Pharmingen) and DAPI were added for 15 min at room temperature in the dark, followed by adding 800 uL binding buffer. Samples were analyzed on a BD Fortessa cytometer within 1 hour.

### Viral Transduction of LSK Cells and Transplantation

For LSK lentiviral infection and rescue BMT, sorted LSK cells from either Hectd1^{f/f} or Hectd1^{f/f;Vav} (CD45.2) mice were cultured in SFEM media (StemCell Technologies Inc) supplemented with 10% FBS (SAFC Biosciences) and cytokines (100 ng/mL mSCF, 20 ng/mL mTpo, 20 ng/mL FLT3L, 20 ng/mL IL6) for 2 days. Lentivirus carrying mCherry/shLuc or mCherry/shmZNF622 were preloaded twice into a RetroNectin (T100B, Takara)-coated 12-well plate (Modlich et al., 2009). Cultured LSKs were transferred to the lentivirus-preload plates and incubated for one more day. At day3, 250k cultured LSKs were mixed with 500k Sca1-depleted competitor BM cells and injected into lethally-irradiated recipient mice. A small fraction of infected cells was spared for flow cytometry to evaluate the viral infection efficiency (Jiang et al., 2012).

### Immunoprecipitation (IP)

For each anti-HA immunoprecipitation, ten ˜80% confluent 10 cm dishes of 293T cells were transiently transfected with pCMV-HA empty vector (control) or pCMV-HA-HectD1. 48 hours after transfection, cells were lysed with IP buffer (10 mM Tris, pH7.4, 150 mM NaCl, 0.5% NP-40, 1 mM NaF, 1 mM Na₃VO₄, PMSF, protease inhibitor cocktail, 10 μM PR-619 (LifeSensors), 4 mM 1,10-phenanthroline (o-PA; Mallinckrodt Chemicals), 4 mM N ethylmaleimide (NEM; Sigma-Aldrich)) for 30 min at 4° C. Cell lysates were clarified by centrifugation at 13,000 rpm for 10 min at 4° C., and then pre-cleared with protein A/G beads for 30 min.

HA-EZ Agarose beads (E6779, Sigma) were prepared by being sequentially washed with 0.1M pH2.5 Glycine, twice in 1M pH8.0 Tris buffer, and twice in IP buffer. Precleared supernatants were incubated with 100 uL washed HA-EZ Agarose beads for 4 hrs with gentle agitation. We transferred the IPs to BioRad Micro Bio-Spin Chromatography Columns (Cat #732-6204), and washed columns with 1 mL IP buffer for four times, followed by a quick spin down to drain the leftover IP buffer. We then seal the bottom of the columns with parafilm, and added 50 uL 1 mg/mL HA peptides (#26184, Thermo Fisher Scientific) for 15 min at 30° C. with occasional mixing, and collected the elute as “HA elutel”. This step was repeated to get another 50 uL HA peptide eluate as “HA elute2”. Then the beads were incubated with 50 uL of 0.1M pH2.5 Glycine for 5 min at room temperature twice to get “Glycine elute 1 and 2”, which was neutralized with 5 uL pH8.0 Tris. Lastly, the beads were boiled in 75 uL 1*LDS loading buffer. All elutes from above were added with 25 uL 3*LDS loading buffer and boiled for 5 min. A small aliquot of eluted samples was resolved with SDS-PAGE, and evaluated by silver staining. Once determined the purification was successful, we loaded majority of the first HA eluates on an SDS-PAGE, stained with colloidal blue. Gel slices were excised and subsequently subjected to mass spectrometry analysis at the Harvard Taplin Mass Spectrometry facility.

### Mass Spectrometry and Significance Analysis of INTeractome (SAINT)

Excised gel bands were cut into approximately 1 mm³ pieces. Gel pieces were then subjected to a modified in-gel trypsin digestion procedure (Shevchenko et al., 1996). Gel pieces were washed and dehydrated with acetonitrile for 10 min, followed by removal of acetonitrile. Pieces were then completely dried in a speed-vac. Rehydration of the gel pieces was with 50 mM ammonium bicarbonate solution containing 12.5 ng/μl modified sequencing-grade trypsin (Promega, Madison, WI) at 4° C. After 45 min, the excess trypsin solution was removed and replaced with 50 mM ammonium bicarbonate solution to just cover the gel pieces. Samples were then placed in a 37° C. room overnight. Peptides were later extracted by removing the ammonium bicarbonate solution, followed by one wash with a solution containing 50% acetonitrile and 1% formic acid. The extracts were then dried in a speed-vac (˜1 hr). The samples were then stored at 4° C. until analysis.

On the day of analysis, the samples were reconstituted in 5-10 μl of HPLC solvent A (2.5% acetonitrile, 0.1% formic acid). A nano-scale reverse-phase HPLC capillary column was created by packing 2.6 μm C18 spherical silica beads into a fused silica capillary (100 μm inner diameter x ˜30 cm length) with a flame-drawn tip (Peng and Gygi, 2001). After equilibrating the column each sample was loaded via a Famos auto sampler (LC Packings, San Francisco CA) onto the column. A gradient was formed and peptides were eluted with increasing concentrations of solvent B (97.5% acetonitrile, 0.1% formic acid).

Eluted peptides were subjected to electrospray ionization and then entered into an LTQ Orbitrap Velos Pro ion-trap mass spectrometer (Thermo Fisher Scientific, Waltham, MA). Peptides were detected, isolated, and fragmented to produce a tandem mass spectrum of specific fragment ions for each peptide. Peptide sequences (and hence protein identity) were determined by matching protein databases with the acquired fragmentation pattern by the software program, Sequest (Thermo Fisher Scientific, Waltham, MA) (Eng et al., 1994). All databases include a reversed version of all the sequences and the data was filtered to between a one and two percent peptide false discovery rate.

We performed 3 biological replicates of IP-MS. Three replicates were evaluated by CRAPome (v1.1) (Contaminant Repository for Affinity Purification) analysis (http://www.crapome.org/) (Mellacheruvu et al., 2013). We used five HA controls with four (CC51, CC52, CC53, CC54) from CRAPome repository and one from our own HA control. To calculate SAINT scores (Choi et al., 2012), spectral counts were analyzed using SAINT parameters “Lowmode=0, MinFold=0, Normalize=1”. The presented SAINT score was the average probability of SAINT results from all three biological replicates. Fold change (FC) is the ratio of the normalized spectral counts of a potential HectD1-interactor to the average of three highest normalized spectral counts of that protein across the negative controls. We chose the cutoff of SAINT score ≥0.5 and fold change (FC) ≥3 based on the established HectD1-interactors (RIOK2, (Varjosalo et al., 2013); ZRANB1, (Tran et al., 2013); SMC2, (Li et al., 2015)). ZNF622 was among the top hits of all 3 replicates (Supplemental Excel). Gene Ontology (GO) analysis of HectD1-interacting proteins was performed using PANTHER Classification System (https://pantherdb.org) (Mi et al., 2019).

### Polysome Profiling

20˜30 million TF-1 cells with shLuc or shHECTD1 were pre-treated with 100 ug/mL cycloheximide (CHX) for 5 min and washed with ice-cold CHX-containing PBS. After centrifugation, cell pellets were lysed in polysome lysis buffer (20 mM Tris, pH7.5, 1.5 mM MgCl₂, 140 mM KCl, 1% Triton X-100, 100 ug/mL CHX, 0.5 mM DTT, protease inhibitor cocktail) for 10 min in ice with gentle rocking. The cell lysate was clarified by centrifugation at 17,000g for 10 min at 4° C. OD₂₆₀ value was measured in Nanodrop. Linear sucrose gradient (7%-45%) was generated using a Gradient Maker (BioComp Instruments, Canada). 15-20 OD₂₆₀ of total cell extract was loaded on the sucrose gradient, followed by ultracentrifugation at 35,000 rpm for 3 hrs 20 min at 4° C. in SW40 rotor. Polysome profiling was analyzed with a BioComp fractionator.

For detection of protein distribution, a total of 13 fractions (830 uL/fraction) from polysome profiling were collected by a fraction collector (Cat #4422151, FC-203B, Gilson). To extract proteins for Western Blot (WB) analysis, 150 uL of each fraction was pelleted by methanol-choloform-H₂O precipitation with sequential addition of 600 uL ice-cold methanol, 225 uL chloroform and 450 uL H₂O. The reaction was thoroughly mixed by inversion and centrifuged at 20,000g for 4 min at 4° C. After carefully removing the aqueous layer, 1 mL prechilled methanol was added and mixed by inversion, followed by centrifugation at 20,000g for 4 min at 4° C. The supernatant was then decanted, and the protein pellet was dried at room temperature. 50 uL 1⁺LDS loading solution was directly added to dissolve protein pellets by frequent pipetting up and down. Due to the extra abundance of proteins in cytoplasmic fractions 1-3, 2 uL of fractions 1-3 along with 20 uL of fractions 4-13 (different ribosomal fractions) were loaded onto an SDS-PAGE for WB analysis.

### Ribosomal Subunit Dissociation and Re-Association Assay

This assay was adapted from a previously published work (Burwick et al., 2012). For ribosomal subunit dissociation, 20˜30 TF-1 cells were harvested without cycloheximide treatment and lysed with low Mg²⁺ buffer (20 mM Tris, pH7.4, 140 mM KCl, 0.25 mM MgCl_{2, 0.5} mM DTT, 1% Triton X-100, EDTA-free protease inhibitor cocktail (Roche)) for 10 min on ice. After clarification by centrifugation at 17,000g for 5 min at 4° C., the cell lysate was divided into two aliquots. One aliquot was loaded on a 7-45% low Mg²⁺ sucrose gradient (20 mM Tris, pH7.4, 140 mM KCl, 0.25 mM MgCl_{2, 0.5} mM DTT, EDTA-free protease inhibitor cocktail (Roche)) and analyzed with a BioComp fractionator to detect total 40S and 60S.

For ribosomal subunit re-association, 2.5M MgCl₂ was added to the other aliquot for a final concentration of 10 mM Mg²⁺and incubated for 5 min at 37° C. The resultant cell lysate was loaded on a 7-45% high Mg²⁺ sucrose gradient (20 mM Tris, pH7.4, 140 mM KCl, 10 mM MgCl₂, 0.5 mM DTT, EDTA-free protease inhibitor cocktail (Roche)) and analyzed with a BioComp fractionator.

Denatured his-Ubiquitination Assay

293T cells were transfected with indicated HectD1 and ZNF622 constructs, as well as His-tagged Ub WT, K48R or K63R mutants. 2 days later, cells were harvested and washed with cold PBS twice, and then lysed with denatured Urea Lysis buffer (100 mM Na₂HPO₄, 100 mM NaH₂PO₄, 10 mM Tris, pH8.0, 0.2% Triton X-100, 5 mM β-Mercaptoethanol, 10 mM Imidazole, 8M urea), followed by immediate vortex and rocking at RT for 20 min. After centrifuging for 10 min at 15,000g, supernatant was obtained and incubated with HisPur Ni-NTA resin (88221 Thermo Scientific) for 2 hrs at RT. Resin was then washed 3 times with wash buffer (100 mM Na₂HPO₄, 100 mM NaH₂PO₄, 10 mM Tris, pH6.3, 0.2% Triton X-100, 5 mM β-Mercaptoethanol, 20 mM Imidazole, 8M urea). Bound ubiquitinated proteins were eluted with SDS elution buffer (200 mM Imidazole, 5% SDS, 150 mM Tris, pH6.8, 30% glycerol, 720 mM 3-Mercaptoethanol) and subjected to western blot analysis.

### Cytokine Signaling, Protein Half-Life Assay and Western Blot (WB)

For cytokine signaling, TF-1/MPL cells were stared in RPMI-1640 media plus 0.5% BSA for 2-4 hrs, and then stimulated with TPO for indicated time points and snap-frozen in dry ice. For mTOR signaling, we also stimulated starved cells with a graded concentration of calf serum. Cell pellets were lysed in LDS loading buffer and sonicated for homogenization. For measuring protein half-life, cycloheximide (CHX) was employed to block de novo protein synthesis for different time points prior to cell harvest.

Protein lysates were subjected to standard WB protocols. Briefly, samples were resolved by SDS-PAGE, and transferred to NC membrane. For all primary phosphor-antibody blots, membranes were blocked with 5% BSA (BP1600-100, Fisher Bioreagents) in TBS-T, while other primary antibody blots were blocked with 5% non-fat milk (sc2325, Santa Cruz). Membranes were incubated with primary antibodies for 2 hrs at room temperature or overnight in cold room. Following primary antibody blots, membranes were washed with TBS-T extensively, and then incubated with HRP-conjugated secondary antibody for 1 hr at room temperature. After extensive washing, membranes were developed with ECL (#34095, Thermo Scientific). To compare immunoblots with a large number of samples that require multiple gels, samples were resolved in SDS-PAGE gels in parallel. Immunoblots were processed and developed side by side, then images were placed side-by-side for presentation.

### HSC Cell Growth and MTT Proliferation Assay

100 LT-HSCs from Hectd1^{f/f} for Hectd1^{f/f;Vav} mice were sorted into round-bottom 96-well plate and cultured in SFEM (StemCell Technologies Inc.) supplemented with 10% FBS and various combinations or concentrations of cytokines as indicated in the main text. At different days, cell numbers were enumerated in the presence of trypan blue using a hemacytometer slide.

TF-1/MPL cells with shLuc or shHECTD1 were cultured in an increasing dose of GM-CSF or TPO in a 96-well plate (10k cells/100 uL per well) for 3 days. 3-(4,5-dimethylthiazole-2-yl)-2,5-diphenyl tetrazolium bromide (MTT; M6494, Invitrogen) was added to media at a final concentration of 0.5 mg/mL for 3-4 hrs at 37° C. Stopping buffer (15% SDS, 2.5% Acetic acid, 50% dimethylformamide) was then added to terminate the reaction. The absorbance was read by a spectrophotometer at 570 nm.

### CD34⁺HSPC Isolation, Culture and Transduction.

Bone marrow (BM) or blood CD34⁺HSPCs were isolated by magnetic separation on an AutoMACS Pro using microbeads conjugated with anti-human CD34 antibodies (Miltenyi). CD34⁺HSPCs were cultured in StemSpan SFEM II (Stemcell Technologies) supplemented with 10% FBS, penicillin/streptomycin, L-glutamine, 2-mercaptoethanol, 1 μM SR-1 (Stemcell Technologies), 100 ng/mL SCF, 40 ng/mL FLT3L, 50 ng/mL TPO, 20 ng/mL IL3, 20 ng/mL IL6, and 15 ng/mL GM-CSF (Peprotech Inc. and Stemcell Technologies). For single transduction studies, after overnight culture, HSPCs were seeded onto plates coated in retronectin (Takara Bio) loaded with viral particles and spinoculated at 800×g at 37C for 90 minutes. For sequential transduction studies, second lentiviral transduction was performed a day later. After spinoculation, HSPCs were cultured for an additional 16-20 hours and then collected for xenotransplantation. All transductions were conducted in the presence of Lentiblast (Oz Biosciences) supplemented in culture media at a multiplicity of infection (MOI) of 20-60.

### Colony Forming Cell Assays.

CD34⁺HSPCs were sorted 48 hrs post-transduction for mCherry positivity. Sorted mCherry⁺ HSPCs were either directly plated onto semi-solid methylcellulose media (Methocult H4230, Stemcell Technologies) supplemented with 5 U/mL EPO, 10 ng/mL IL-3, 5 ng/mL SCF, 5 ng/mL GM-CSF, or subjected to an extended in vitro culture for 5 additional days before plating in methylcellulose media. Colonies were enumerated 12-14 days after plating.

### Cd34⁺Hspc Xenotransplantation.

NOD. Cg-Kit^{W-41J}Tyr⁺Prkdc^{scid}II2rg^{fm1Wj1}/ThomJ (NBSGW) mice were originally purchased from Jackson Laboratory (stock #026622)38 and expanded in our barrier facility. 6-8 week-old nonirradiated NBSGW mice were retro-orbitally infused with HSPCs 16-20 hrs post transduction. Alternatively, transduced human HSPCs were injected into buslfan preconditioned NSG mice. Human engraftment in the peripheral blood (PB) was tracked by retro-orbital blood collection and analyzed by flow cytometry. After 16-27 weeks the BMs and spleens were isolated for flow cytometric analysis and secondary xenotransplantation was conducted by infusing 10⁷ total BM cells from primary recipients into NBSGW recipients.

### Quantification and Statistical Analysis

Statistics for MTT, HSC cell growth and CFC assays were performed using unpaired two-tailed Student's t test and error bars indicate mean±SD. Signal intensities of Western blot bands were quantified by Fiji software. Statistics was analyzed using unpaired two-tailed Student's t test. All the relevant quantifications performed in primary mice or transplanted mice were analyzed by GraphPad Prism and error bars indicate mean±SEM. Paired t test was used for pairwise comparison of HSC/MPP numbers in Cyclophosphamide+2GCSF challenged mice. Details of “n” values describing the number of experiment repeats or mice are shown in the figure legends. Statistics significance was determined by Student's ttest. ns, not significant; *: p<0.05; **: p<0.01; ***: p<0.001.

RESOURCES TABLE

| | | |
|---|---|---|
| REAGENT or RESOURCE | SOURCE | IDENTIFIER |

Antibodies

| | | |
|---|---|---|
| Rabbit polyclonal anti-HectD1 | Bethyllab | Cat# A302-908A, RRID:AB_10664800 |
| Rabbit polyclonal anti-ZNF622 | Bethyllab | Cat# A304-075A, RRID:AB_2621324 |
| Rabbit polyclonal anti-NOB1 | Bethyllab | Cat# A304-680A, RRID:AB_2620875 |
| Rabbit polyclonal anti-RPS3 | Bethyllab | Cat# A303-840A, RRID:AB_2620191 |
| Rabbit polyclonal anti-RPL23A | Proteintech | Cat# 16386-1-AP, RRID:AB_2269755 |
| Rabbit polyclonal anti-RPL24 | Proteintech | Cat# 17082-1-AP, RRID:AB_2181728 |
| Rabbit polyclonal anti-elF6 | Proteintech | Cat# 10291-1-AP, RRID:AB_2096515 |
| Rabbit polyclonal anti-NMD3 | Proteintech | Cat# 16060-1-AP, RRID:AB_2282830 |
| Rabbit polyclonal anti-DNAJC21 | Proteintech | Cat# 23411-1-AP, RRID:AB_2879274 |
| Mouse monoclonal anti-PA2G4 | Proteintech | Cat# 66055-1-Ig, RRID:AB_11042597 |
| Rabbit polyclonal anti-RPL11 | Abcam | Cat# ab79352, RRID:AB_2042832 |
| Rabbit monoclonal anti-RPL11 | Abcam | Cat# ab32157, RRID:AB_732117 |
| Mouse monoclonal anti-GSK3α/β | Millipore | Cat# 368662-200UG, RRID:AB_2043310 |
| Mouse monoclonal anti-HSP90 | StressMarq | Cat# SMC-107, RRID:AB_854214 |
| Mouse monoclonal anti-β-Catenin | BD Biosciences | Cat# 610153, RRID:AB_397554 |
| Goat monoclonal anti-Actin | Santa Cruz | Cat# sc-1616, RRID:AB_630836 |
| Rabbit polyclonal anti-STAT5 | Santa Cruz | Cat# sc-835, RRID:AB_632446 |
| Rabbit monoclonal anti-pY1007/ | Cell Signaling | Cat# 3776, RRID:AB_2617123 |
| 1008-JAK2 | | |
| Rabbit monoclonal anti-JAK2 | Cell Signaling | Cat# 3230, RRID:AB_2128522 |
| Rabbit polyclonal pY694-STAT5 | Cell Signaling | Cat# 9351, RRID:AB_2315225 |
| Rabbit polyclonal anti-pS473-AKT | Cell Signaling | Cat# 9271, RRID:AB_32982 |
| Rabbit polyclonal anti-AKT | Cell Signaling | Cat# 9272, RRID:AB_329827 |
| Mouse monoclonal anti-pT202/ | Cell Signaling | Cat# 9106, RRID:AB_331768 |
| 204-ERK1/2 | | |
| Rabbit polyclonal anti-ERK1/2 | Cell Signaling | Cat# 9102, RRID:AB_330744 |
| Rabbit monoclonal anti-pS2448-mTOR | Cell Signaling | Cat# 5536, RRID:AB_10691552 |
| Mouse monoclonal anti-mTOR | Cell Signaling | Cat# 4517, RRID:AB_1904056 |
| Mouse monoclonal pT389-S6K1 | Cell Signaling | Cat# 9206, RRID:AB_2285392 |
| Rabbit polyclonal anti-S6K1 | Cell Signaling | Cat# 9202, RRID:AB_331676 |
| Rabbit monoclonal anti-pS235/ | Cell Signaling | Cat# 4858, RRID:AB_916156 |
| 236-RPS6 | | |
| Rabbit polyclonal anti-pS240/ | Cell Signaling | Cat# 2215, RRID:AB_331682 |
| 244-RPS6 | | |
| Rabbit monoclonal anti-RPS6 | Cell Signaling | Cat# 2217, RRID:AB_331355 |
| Rabbit monoclonal anti-pT37/ | Cell Signaling | Cat# 2855, RRID:AB_560835 |
| 46-4E-BP1 | | |
| Rabbit monoclonal anti-4E-BP1 | Cell Signaling | Cat# 9644, RRID:AB_2097841 |
| Rabbit polyclonal anti-pS21/ | Cell Signaling | Cat# 9331, RRID:AB_329830 |
| 9-GSK3α/β | | |
| Rabbit polyclonal anti-elF2α | Cell Signaling | Cat# 9722, RRID:AB_2230924 |
| Rabbit monoclonal anti-β-Tubulin | Cell Signaling | Cat# 2128, RRID:AB_823664 |
| Mouse monoclonal anti-GAPDH | Cell Signaling | Cat# 97166, RRID:AB_2756824 |
| Rabbit monoclonal anti-HA | Cell Signaling | Cat# 3724, RRID:AB_1549585 |
| Mouse monoclonal anti-FLAG M2-HRP | Sigma-Aldrich | Cat# A8592, RRID:AB_439702 |
| Digital anti-mouse HRP | Kindle Biosciences | Cat# R1005, RRID:AB_2800463 |
| Digital anti-Rabbit HRP | Kindle Biosciences | Cat# R1006, RRID:AB_2800464 |
| CD45.1-FITC | BD Biosciences | Cat# 553775, RRID:AB_395043 |
| CD45.1-PE-Cy7 | Thermo Fisher | Cat# 25-0453-82, RRID:AB_469629 |
| CD45.1-eF450 | Thermo Fisher | Cat# 48-0453-82, RRID:AB_1272189 |
| CD45.2-APC-Cy7 | Thermo Fisher | Cat# 47-0454-82, RRID:AB_1272175 |
| CD45.2-Buv395 | BD Biosciences | Cat# 564616, RRID:AB_2738867 |
| Gr1-PE | Thermo Fisher | Cat# 12-5931-83, RRID:AB_466046 |
| Mac1-APC | Thermo Fisher | Cat# 17-0112-83, RRID:AB_469344 |
| B220-PE | BD Biosciences | Cat# 553090, RRID:AB_394620 |
| B220-APC | Thermo Fisher | Cat# 17-0452-83, RRID:AB_469396 |
| CD4-PE | BD Biosciences | Cat# 553049, RRID:AB_394585 |
| CD8a-PE | BioLegend | Cat# 100707, RRID:AB_312746 |
| CD3e-FITC | Thermo Fisher | Cat# 11-0031-82, RRID:AB_464882 |
| CD3e-PE | Thermo Fisher | Cat# 12-0031-85, RRID:AB_465498 |
| Ter119-biotin labeled | Thermo Fisher | Cat# 13-5921-85, RRID:AB_466798 |
| Gr1-biotin labeled | Thermo Fisher | Cat# 13-5931-86, RRID:AB_466802 |
| Mac1-biotin labeled | Thermo Fisher | 13-0112-86, RRID:AB_466361 |
| B220-biotin labeled | Thermo Fisher | Cat# 13-0452-86, RRID:AB_466451 |
| CD19-biotin labeled | Thermo Fisher | Cat# 13-0193-86, RRID:AB_657655 |
| CD4-biotin labeled | Thermo Fisher | Cat# 13-0041-86, RRID:AB_466327 |
| CD5-biotin labeled | Thermo Fisher | Cat# 13-0051-85, RRID:AB_466340 |
| CD8a-biotin labeled | Thermo Fisher | Cat# 13-0081-86, RRID:AB_466348 |
| Streptavidin-PE-TexasRed | Thermo Fisher | Cat# SA1017, RRID:N/A |
| Streptavidin-APC-Cy7 | Thermo Fisher | Cat# 47-4317-82, RRID:AB_10366688 |
| c-Kit-PE | BD Biosciences | Cat# 553355, RRID:AB_394806 |
| c-Kit-APC | Thermo Fisher | Cat# 17-1171-83, RRID:AB_469431 |
| c-Kit-APC-Cy7 | Thermo Fisher | Cat# 47-1171-82, RRID:AB_1272177 |
| Scal-PE | BD Biosciences | Cat# 553336, RRID:AB_394792 |
| Scal-PerCp-Cy5.5 | Thermo Fisher | Cat# 45-5981-82, RRID:AB_914372 |
| CD150-PE-Cy7 | BioLegend | Cat# 115914, RRID:AB_439797 |
| CD48-FITC | BioLegend | Cat# 103403, RRID:AB_313018 |
| CD48-APC-Cy7 | BioLegend | Cat# 103432, RRID:AB_2561463 |
| CD48-AF700 | BioLegend | Cat# 103426, RRID:AB_10612755 |
| CD48-APC | Thermo Fisher | Cat# 17-0481-82, RRID:AB_469408 |
| Flk2-PE | BD Biosciences | Cat# 553842, RRID:AB_395079 |
| CD34-APC | Thermo Fisher | Cat# 50-0341-82, RRID:AB_10596826 |
| CD16/32-PE | BD Biosciences | Cat# 553145, RRID:AB_394660 |

Bacterial and Virus Strains

| | | |
|---|---|---|
| pCL20.MSCV.mir30.PGK.mCherry | Holmfeldt et al., 2016 | N/A |
| pCL20.MSCV.mir30.PGK.GFP | This paper (Replace | N/A |
| | mCherry with GFP) | |

Chemicals, Peptides, and Recombinant Proteins

| | | |
|---|---|---|
| 5-Fluorouracil (5-FU) | Sigma-Aldrich | Cat# F6627 |
| Cyclophosphamide | Baxer | Cat# NDC 10019-955-01 |
| O-Propargyl-Puromycin (OP-Puro) | MedChem Express | Cat# HY-15680/CS-6850 |
| BrdU | BD Pharmingen | Cat# 5133284 |
| Puromycin dihydrochloride | Sigma-Aldrich | Cat# P7255 |
| Insulin Solution Human | Sigma-Aldrich | Cat# 19278 |
| 10% BSA in IMDM | StemCell Technologies | Cat# 09300 |
| L-Glutamine | Gibco | Cat# 25030-081 |
| Holo-Transferrin human | Sigma-Aldrich | Cat# T0665 |
| B-Mercaptoethanol | Sigma-Aldrich | Cat# M3148 |
| Propidium iodide (PI) | Sigma-Aldrich | Cat# P4170 |
| DAPI | Sigma-Aldrich | Cat# D9542 |
| 3% Acetic Acid with Methylene Blue | StemCell Technologies | Cat# 07060 |
| Retronectin | Takara | Cat# T100B |
| Cycloheximide | Sigma-Aldrich | Cat# C7698 |
| Imidazole | Acros Organics | Cat# 288-32-4 |
| N-Ethylmaleimide (NEM) | Sigma-Aldrich | Cat# E1271 |
| PR619 | LifeSensors | Cat# S19619 |
| 1,10-phenanthroline | Mallinckrodt Chemicals | Cat# 2631-55 |
| Sodium Fluoride | Sigma-Aldrich | Cat# S7920 |
| Sodium orthovanadate | Sigma-Aldrich | Cat# 450243 |
| PMSF | Sigma-Aldrich | Cat# P7626 |
| Protease Inhibitor Cocktail Tablets; | Roche | Cat# 11836170001 |
| EDTA-free | | |
| Protease Inhibitor Cocktail Tablets | Roche | Cat# 11697498001 |
| Bovine Serum Albumin (BSA) | Fisher Bioreagents | Cat# BP1600 |
| Nonfat milk | Santa Cruz | Cat# sc2325 |
| StemSpan SFEM | StemCell Technologies | Cat# 09600 |
| Fetal Bovine Serum | SAFC Biosciences | Cat# 12103C |
| Bovine Calf Serum | HyClone | Cat# SH30072.03 |
| HA peptide | Thermo Fisher | Cat# 26184 |
| Anti-HA Affinity Gel | Sigma-Aldrich | Cat# E6779 |
| Anti-FLAG M2 Affinity Gel | Sigma-Aldrich | Cat# F2426 |
| Protein A Sepharose CL-4B | GE Health | Cat# 17-0780-01 |
| Protein G Sepharose 4 Fast Flow | GE Health | Cat# 17-0618-01 |
| HisPur Ni-NTA Resin | Thermo Fisher | Cat# 88221 |
| Murine SCF | Peprotech | Cat# 250-03 |
| Murine Tpo | Peprotech | Cat# 315-14 |
| Murine IL3 | Peprotech | Cat# 213-13 |
| Murine FLT3L | Peprotech | Cat# 250-31L |
| Murine IL6 | Peprotech | Cat# 216-16 |
| rh EPO | Espogen | Cat# NDC 55513-144-10 |
| rG-CSF (Neupogen) | Amgen | Cat# NDC 55513-209-01 |
| Human GM-CSF | Peprotech | Cat# 300-03 |
| Human TPO | Peprotech | Cat# 300-18 |

Critical Commercial Assays

| | | |
|---|---|---|
| Lineage Cell Depletion Kit mouse | Miltenyi Biotec | Cat# 130-090-858 |
| Anti-Sca-1 Microbead Kit (FITC) | Miltenyi Biotec | Cat# 130-092-529 |
| Click-iT Plus OP-Puro Protein | Thermo Fisher | Cat# C10458 |
| Synthesis Kit | | |
| FITC Annexin V Apoptosis Detection | BD Pharmingen | Cat# 556547 |
| Kit | | |
| BrdU Flow Kits | BD Pharmingen | Cat# 557891 |
| BD Cytofix/Cytoperm | BD Biosciences | Cat# 554714 |
| SYBR Green Master Mix | Applied Biosystems | Cat# 43-091-55 |
| RNeasy Plus Mini Kit | QIAGEN | Cat# 74136 |
| qScript cDNA Supermix | Quanta Biosciences | Cat# 98047 |

Experimental Models: Cell Lines

| | | |
|---|---|---|
| TF-1 | ATCC | Cat# CRL-2003, RRID:CVCL_0559 |
| HEK293T | ATCC | Cat# CRL-3216, RRID:CVCL_0063 |
| WEHI-3B | Dr. Harvey Lodish lab | RRID:CVCL_2239 |
| BaF3 | DSMZ | Cat# ACC-300, RRID:CVCL_0161 |

Experimental Models: Mouse Strains

| | | |
|---|---|---|
| C57B6/NGpt-Hectd1^{tm1b}/Gpt | EuComm and MARC, | Cat# 5757216, RRID:MGI:5757216 |
| | China | |
| FLP B6;SJL-Tg(ACTFLPe)9205Dym/J | Jackson laboratories | Cat# JAX:003800, |
| | | RRID: IMSR_JAX:003800 |
| Vav-Cre mice | Stadtfeld and Graf, | N/A |
| | 2005; Chen et al., 2009 | |
| C57BL/6J (CD45.2) | Jackson laboratories | Cat# 000664 |
| SJL (CD45.1) | Jackson laboratories | Cat# 000686 |

Oligonucleotides

| | | |
|---|---|---|
| sh human HECTD1#1: | This paper | N/A |
| tatgaaacaagattgtagtcaa | | |
| sh human HECTD1#2: | This paper | N/A |
| taccactggttgttcaactcta | | |
| sh human ZNF622#1: | This paper | N/A |
| atcggaaagtggagatgatgaa | | |
| sh human ZNF622#2: | This paper | N/A |
| tggagacgattgggaagatatt | | |
| sh mouse Znf622#1: | This paper | N/A |
| agagaaagttggtgttggcaaa | | |
| sh mouse Znf622#2: | This paper | N/A |
| tgtgacagttgctaggaatcaa | | |
| Oligonucleotides for mouse | This paper | N/A |
| genotyping and real-time | | |
| qPCR see Table S2 | | |

Recombinant DNA

| | | |
|---|---|---|
| pCMV-HA-HectD1 WT | Sarkar and Zohn, 2012 | N/A |
| pCMV-HA-HectD1 C2579G | Sarkar and Zohn, 2012 | N/A |
| pCMV-HA-HectD1 truncates/ | This paper | N/A |
| deletion mutants | | |
| pOZ-FH-ZNF622 full length | This paper | N/A |
| pOZ-FH-ZNF622 truncates/ | This paper | N/A |
| deletion mutants | | |
| pOZ-Flag-ZNF622 | This paper | N/A |
| Flowjo | https://www.flowjo.com/ | RRID:SCR_008520 |
| | solutions/flowjo | |
| GraphPad Prism | http://www.graphpad.com/ | RRID:SCR_002798 |
| Fiji | http://fij.sc | RRID:SCR_002285 |
| QuantStudio Real-Time PCR | Thermo Fisher | https://www.thermofisher.com/us/ |
| software | | en/home/life-science/pcr/real- |
| | | time-pcr/ |
| SAINT | Choi et al. 2012 | http://saint-apms.sourceforge.net/ |
| | | Main.html |
| ELDA | Hu and Smyth, 2009 | http://bioinf.wehi.edu.au/ |
| | | software/elda/ |
| Sequest | Eng et al., 1994 | http://tools.thermofisher.com/ |
| | | content/sfs/manuals/Man-XCALI- |
| | | 97160-SEQUEST-331-User- |
| | | ManXCALI97160-B-EN.pdf |

The following examples are provided to illustrate certain embodiments of the invention. They are not intended to limit the invention in any way.

### Example I

Impaired ribosome function is the underlying etiology in a group of bone marrow failure syndromes called ribosomopathies. However, how ribosomes are regulated remains poorly understood, as are approaches to restore hematopoietic stem cell (HSC) function attributable to defective ribosome biogenesis. Here we uncover a previously-unappreciated role for the E3 ubiquitin ligase HectD1 in regulating HSC function via ribosome assembly and protein translation. Hectd1-deficient HSCs exhibit a striking defect in transplantation ability and ex vivo maintenance, concomitant with a reduced protein synthesis and growth rate under stress conditions. Mechanistically, HectD1 ubiquitinates and degrades ZNF622, an assembly factor for the ribosomal 60S subunit. HectD1 loss led to an accumulation of ZNF622 and the anti-association factor eIF6 on the 60S, resulting in 60S/40S joining defects. Importantly, Znf622 depletion in Hectd1-deficient HSCs restored ribosomal subunit joining, protein synthesis, and HSC reconstitution capacity. These findings highlight the importance of ubiquitin-coordinated ribosome assembly in HSC regeneration.

### Generation of a Conditional Hectd1 Knockout Mice in Hematopoietic Cells.

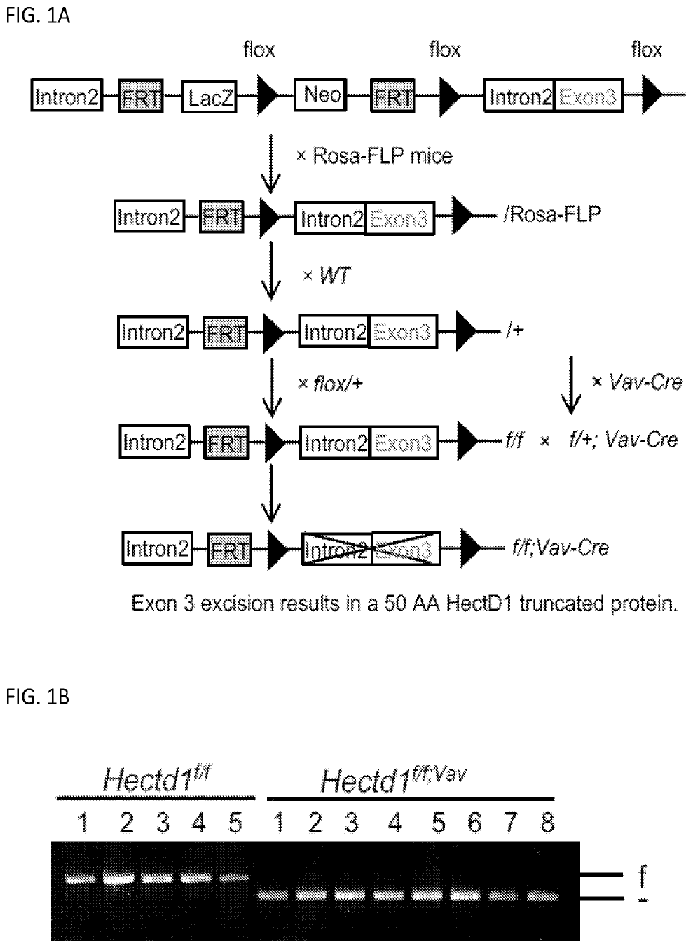

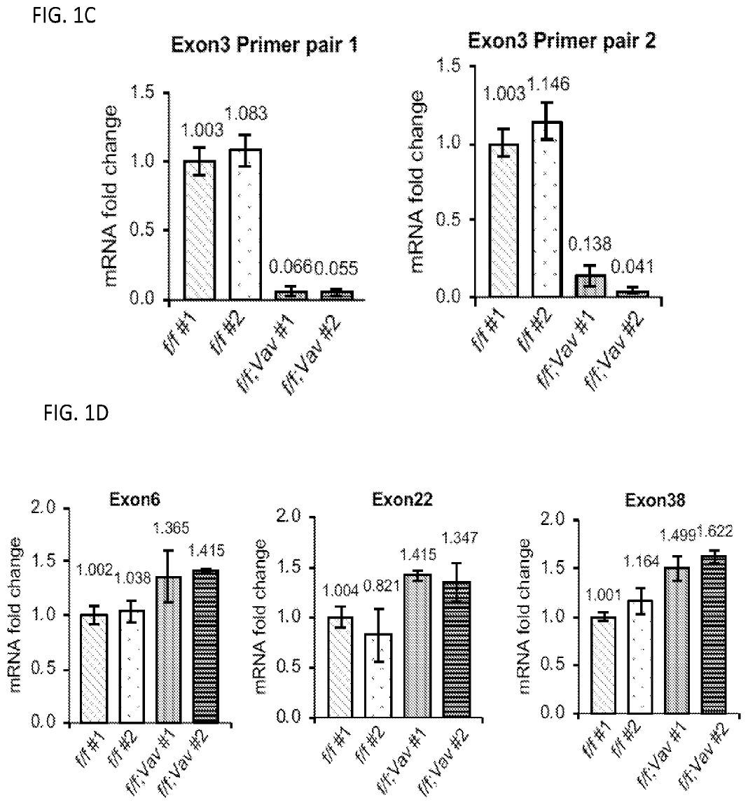

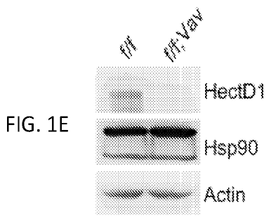

Hectd1 germline knockout (KO) leads to mouse embryonic lethality due to defects in neural tube closure and impaired placenta development (D'Alonzo et al., 2019; Sarkar et al., 2014; Zohn et al., 2007). To explore a potential role for HectD1 in hematopoiesis, we studied a conditional Hectd1 knockout (cKO) mouse model (FIG. 1A). The floxed alleles of Hectd1 that target exon 3 (Hectd1f) were crossed with Vav-cre transgenic mice, in which the Cre recombinase is under the control of the Vav promoter to allow pan-hematopoietic excision of Hectd1 in all hematopoietic cells (Hectd1^{f/f;Vav}). Deletion of exon 3 is predicted to generate an early stop codon in exon 4, thus producing a non-functional 50 amino acid truncated protein. To evaluate deletion efficiency, we plated bone marrow (BM) cells from Hectd1^{f/f} and Hectd1^{f/f;Vav} mice into semisolid methylcellulose culture and confirmed a nearly 100% deletion efficiency by genotyping individual colonies (FIG. 1B). Quantitative PCR analysis (qRT-PCR) demonstrated the specific targeting of exon 3 of the Hectd1 gene (FIGS. 1C and 1D). Moreover, HectD1 protein was undetectable in Hectd1^{f/f;Vav} BM cells (FIG. 1E). Together, these data suggest that Hectd1 was efficiently and specifically deleted in all hematopoietic cells in our cKO mouse model.

### Hectd1 Deficiency Impairs HSC Repopulation Capacity and Decreases Functional HSC Frequency

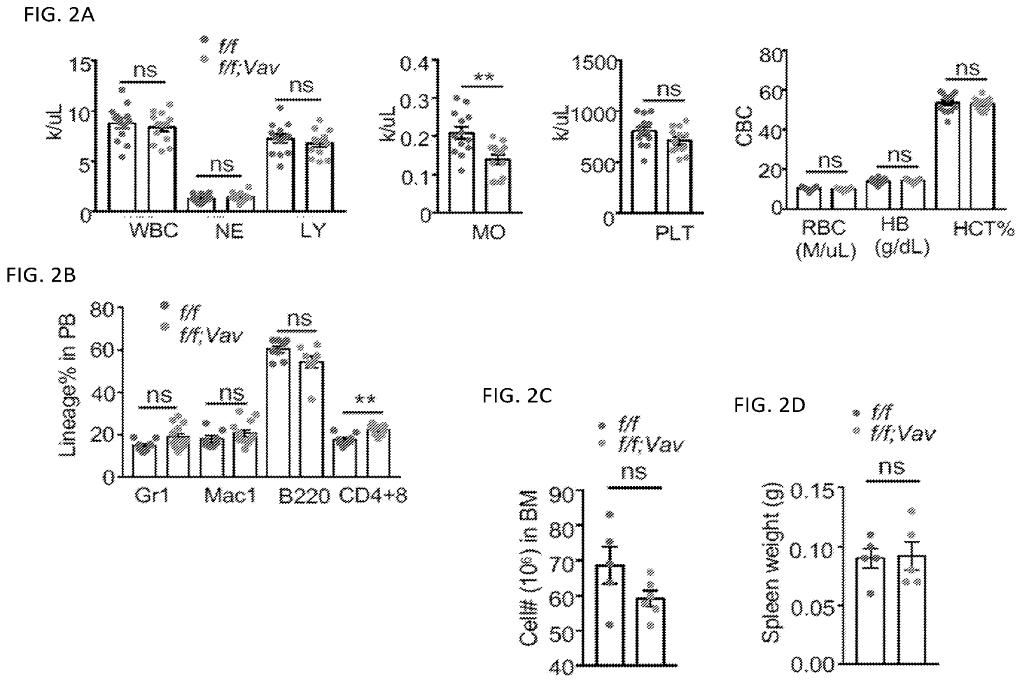

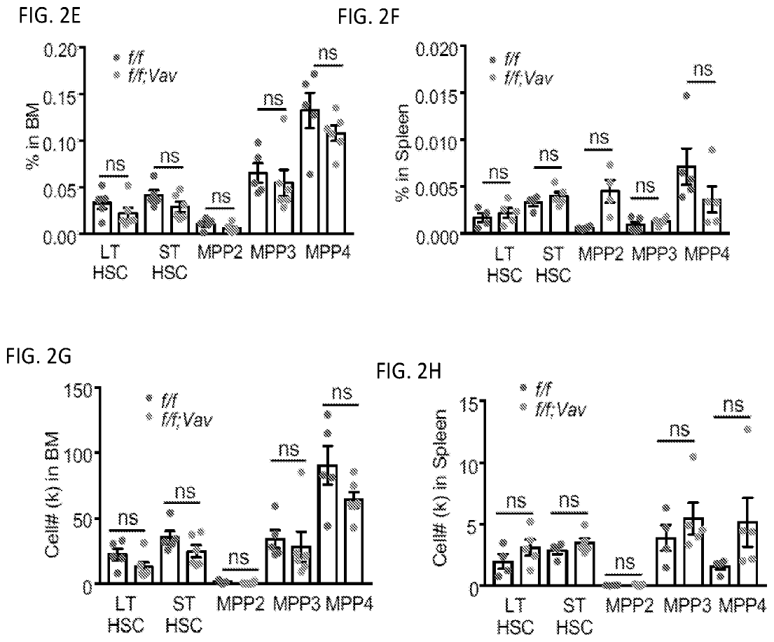

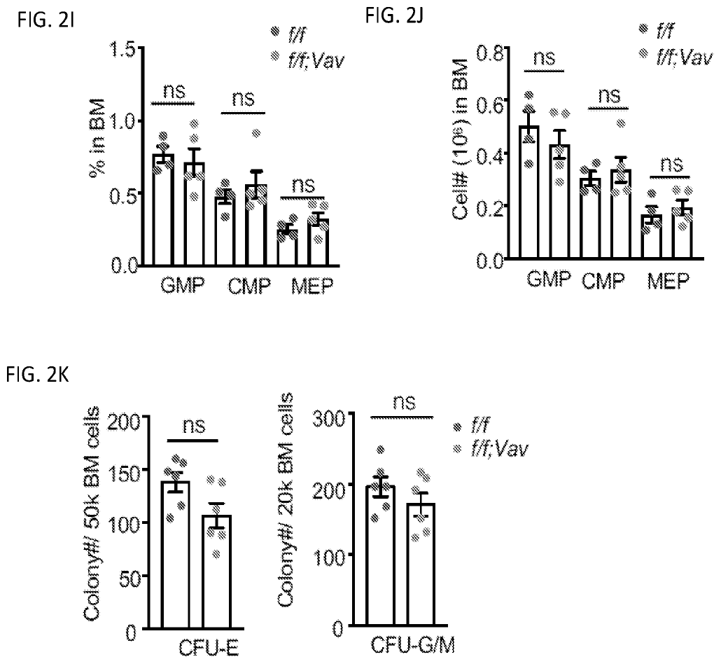

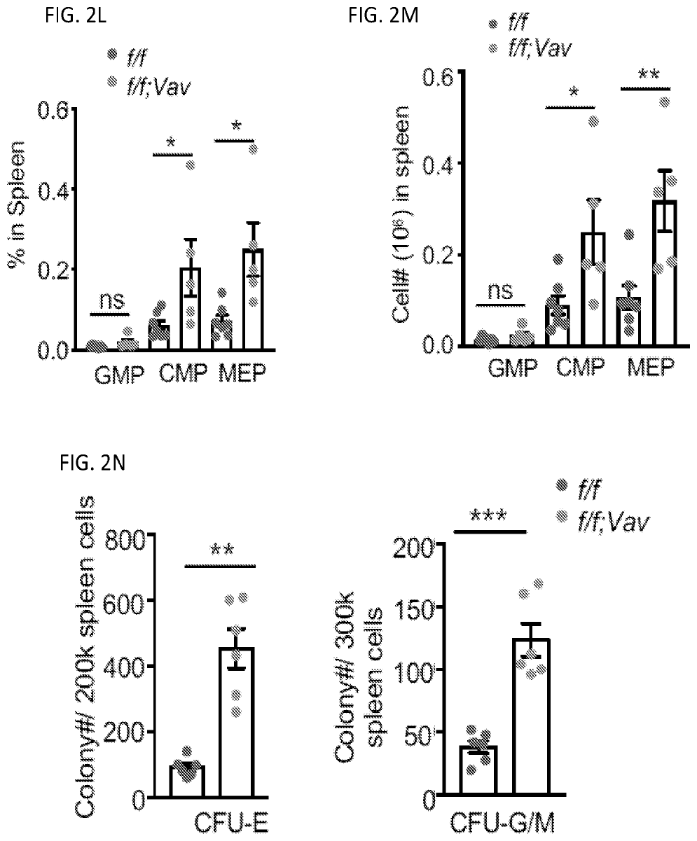

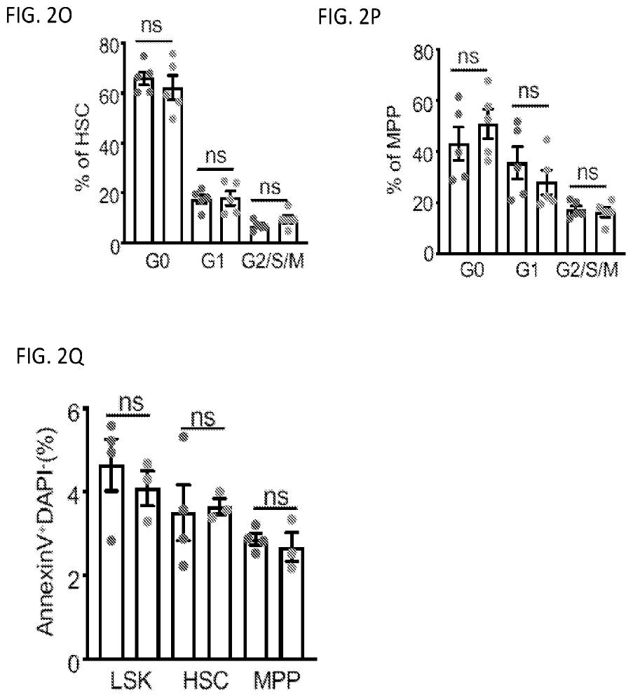

To elucidate the impact of Hectd1 loss on homeostatic hematopoiesis, we first investigated the hematological parameters in young adult mice. Complete blood count (CBC) analysis and flow cytometry of different lineages of blood cells from Hectd1^{f/f;Vav} mice showed largely normal blood counts compared with their Hectd1^{f/f} littermates (FIGS. 2A and 2B). Total BM cellularity and spleen weight were also indistinguishable from controls (FIGS. 2C and 2D). We then characterized the hematopoietic stem and progenitor cells (HSPCs) in the BM and spleen. Hectd1^{f/f;Vav} mice showed comparable HSPC frequencies and numbers to those of Hectd1^{f/f} controls (FIGS. 2E-2H), ie, long-term stem cells (LT-HSCs, Lin⁻Sca1⁺c-Kit⁺Flk2⁻CD150⁺CD48⁻), short-term stem cells (ST-HSCs, Flk2⁻CD150⁻CD48⁻LSK), various multipotent progenitors (MPPs) (Lv et al., 2017). Furthermore, we observed no difference in the frequency and number of committed progenitor cells in the BM by flow cytometry (FIGS. 21 and 2J) or functional progenitors by colony-forming-cell (CFC) assay (Figure S2K). Interestingly, the spleen of Hectd1^{f/f;Vav} mice displayed a significant increase in CMP and MEP progenitors (FIGS. 2L and 2M), and accordingly, more CFU-E and CFU-GM colonies as compared to their Hectd1^{f/f} littermates (FIG. 2N). Moreover, the cell cycle status or apoptosis in HSCs and MPPs remained unchanged (FIGS. 2O-2Q). These observations indicate relatively normal hematopoiesis in young adult Hectd1^{f/f;Vav} mice under homeostatic conditions.

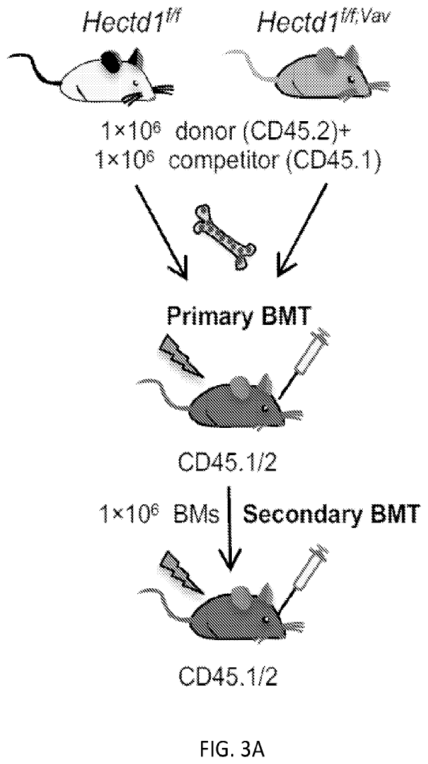

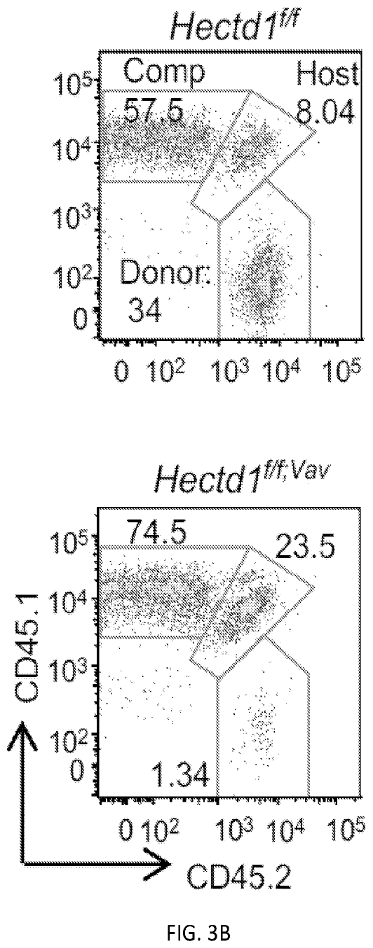

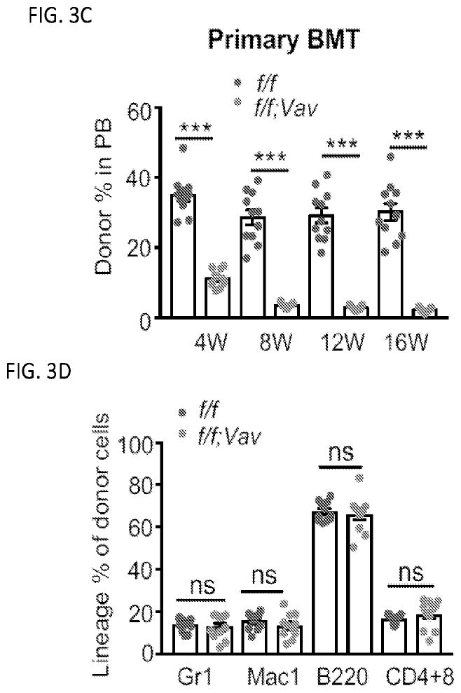

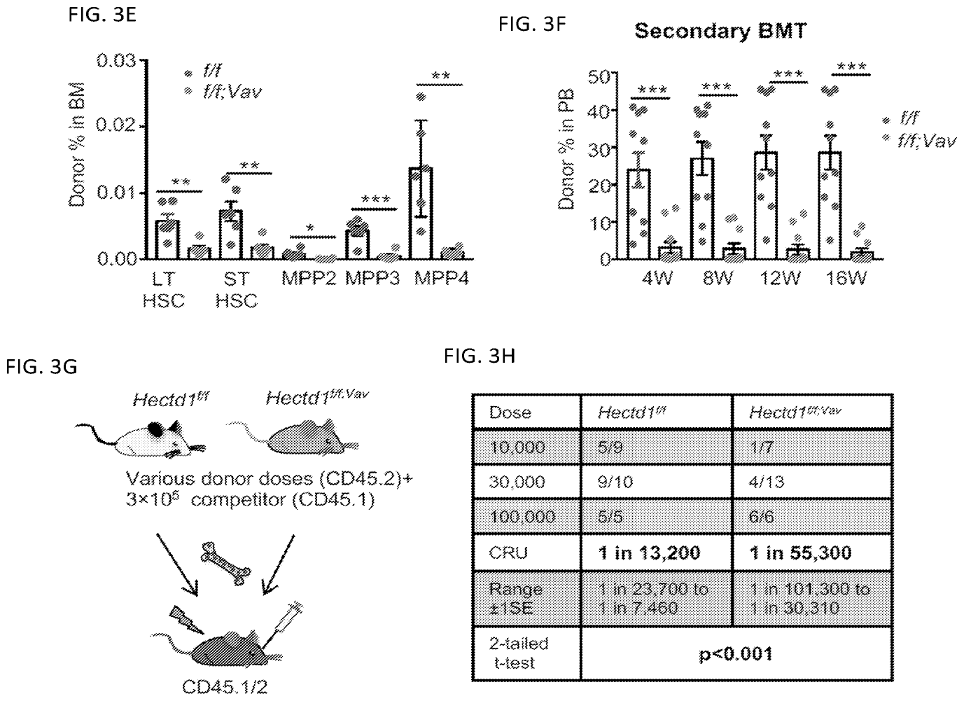

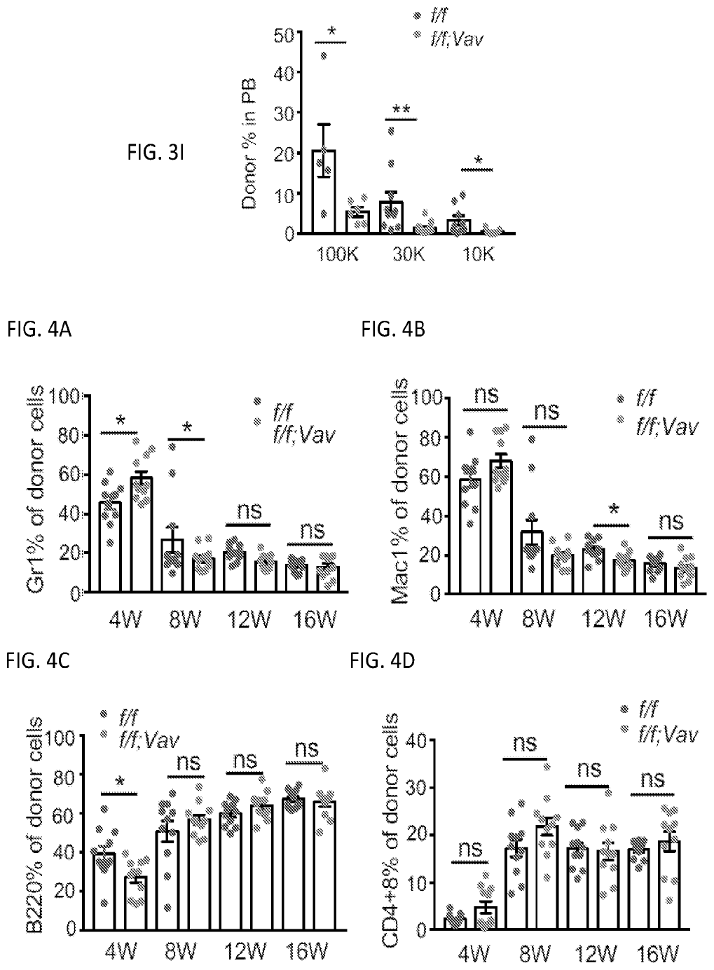

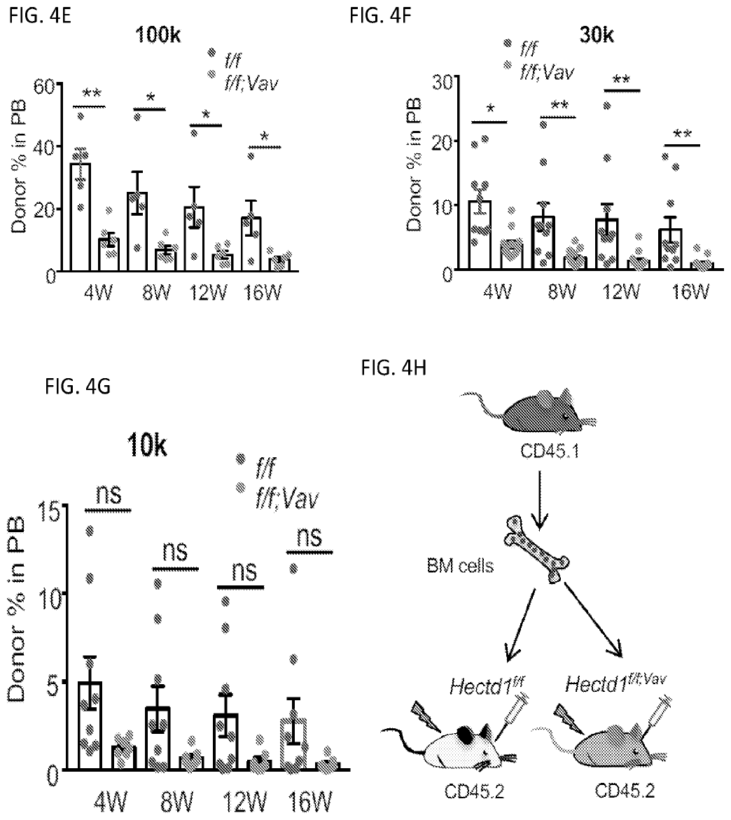

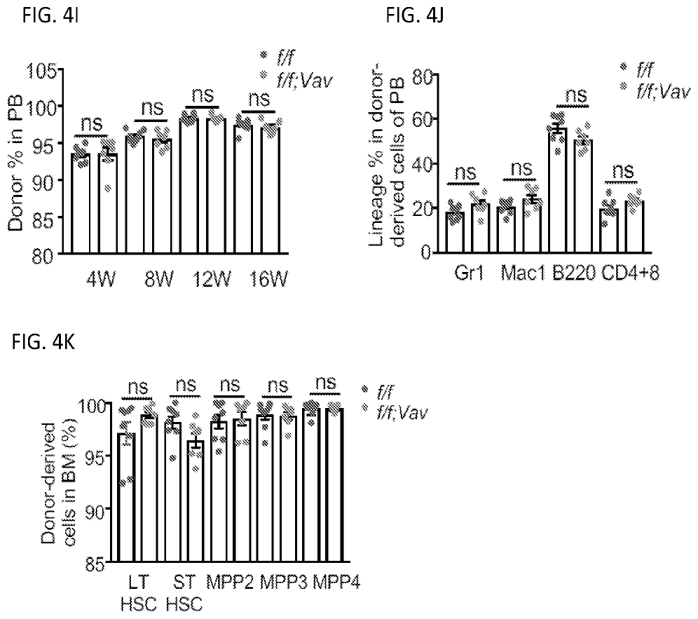

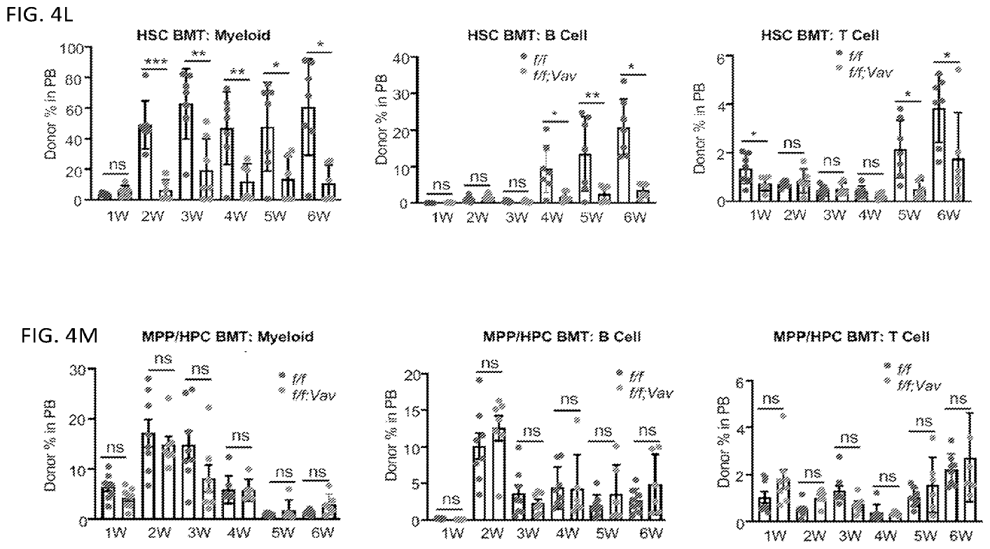

To assess the consequences of Hectd1 loss in HSC function in vivo, we performed competitive BM transplantation (BMT) assay by injecting 1×10⁶ total BM cells from Hectd1^{f/f} or Hectd1^{f/f;Vav} donor mice (CD45.2) with an equal number of BM cells from competitor mice (CD45.1) into lethally irradiated recipients (CD45.1/CD45.2) (FIG. 3A). Donor chimerism in the peripheral blood (PB) of the recipients was determined by flow cytometry every 4 weeks post-BMT. Of note, donor-derived cell percentages from Hectd1^{f/f;Vav} mice were considerably decreased compared with control mice as early as 4 weeks, and exhibited a progressive decline over time (FIGS. 3B and 3C), indicating a defect in LT-repopulating HSCs. Hectd1 loss did not affect lineage distribution in the transplanted recipients (FIGS. 3D and 4A-4D). Importantly, the percentages of donor-derived HSPC populations from Hectd1^{f/f;Vav} mice were substantially decreased in comparison to Hectd1^{f/f} controls (FIG. 3E), indicating a reduction in HSC reconstitution. To further examine the impact of Hectd1 loss on HSC self-renewal, we performed secondary BMT by injecting 1×10⁶ total BM cells from primary transplanted mice into secondary recipients. Defective reconstitution from Hectd1^{f/f;Vav} mice was further exacerbated in secondary transplantation (FIG. 3F). Thus, these data indicate that Hectd1^{f/f;Vav} mice have inferior long-term BM repopulating HSCs with reduced self-renewal.

To functionally quantify HSC frequency, we next employed limiting dilution BMT (FIG. 3G). A graded dose of BM cells from Hectd1^{f/f} and Hectd1^{f/f;Vav} mice mixed with a fixed number of competitor cells were transplanted into lethally irradiated recipient mice. 16 weeks after BMT, mice with donor percentage higher than 1% were defined as “positive reconstitution” (Bersenev et al., 2008). We found that Hectdf1^{f/f;Vav} mice harbored a 4-fold reduction in functional HSC frequencies in the BM by ELDA analysis (extreme limiting dilution analysis) (FIG. 3H) (Hu and Smyth, 2009). Hectd1^{f/f;Vav} BM cells exhibited consistently reduced donor chimerisms in the recipients transplanted at all doses of donor cells (FIGS. 31 and 4E-4G). Since HectD1 was reported to be involved in protein secretion and cell migration (Duhamel et al., 2018), we asked if Hectd1 loss could affect BM niche or in a cell extrinsic manner. Therefore, we performed reciprocal BMT by injecting BM cells from wildtype mice (CD45.1) into Hectd1^{f/f} or Hectd1^{f/f;Vav} mice as recipients (CD45.2) (FIG. S4H). Donor chimerism as well as lineage distribution in the PB and HSPCs in the BM were comparable among recipients of both genotypes (FIGS. 4I-S4K). Taken together, our data provide strong evidence that Hectd1 insufficiency reduces frequencies of functional HSCs in the BM.

### Hectd1 Insufficiency Diminishes HSC Reconstituting Activity In Vivo and HSC Growth Ex Vivo

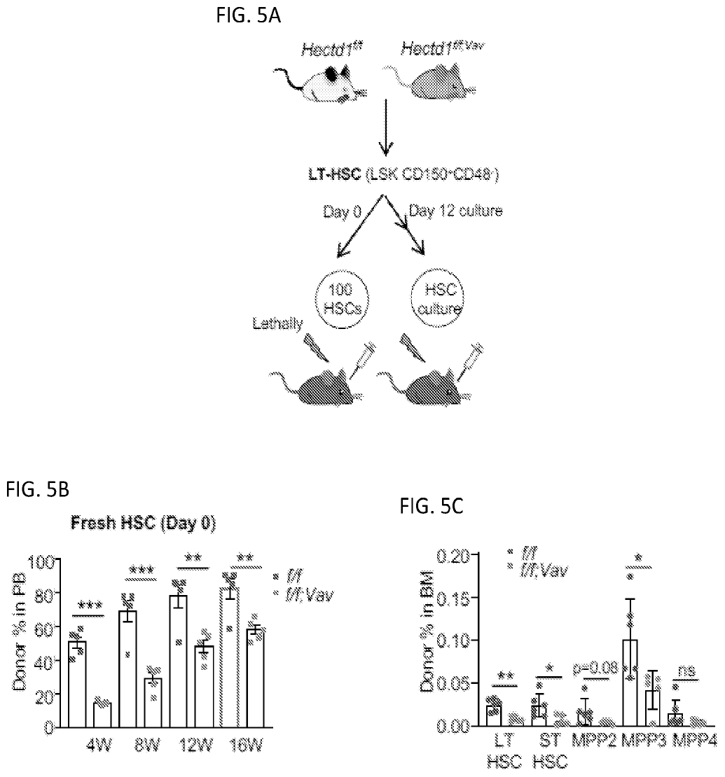

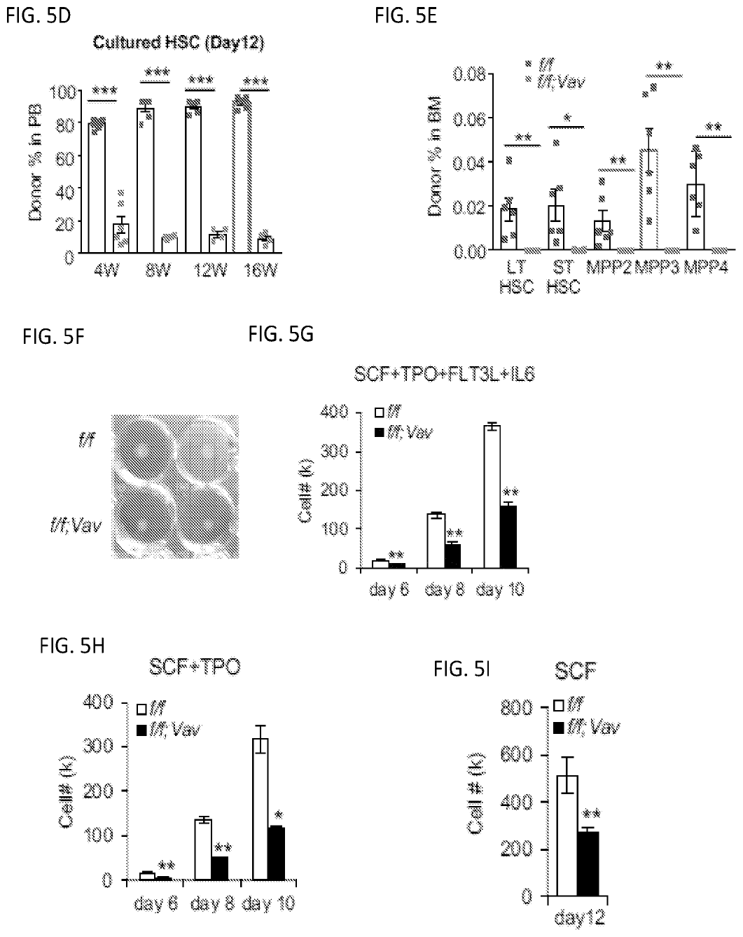

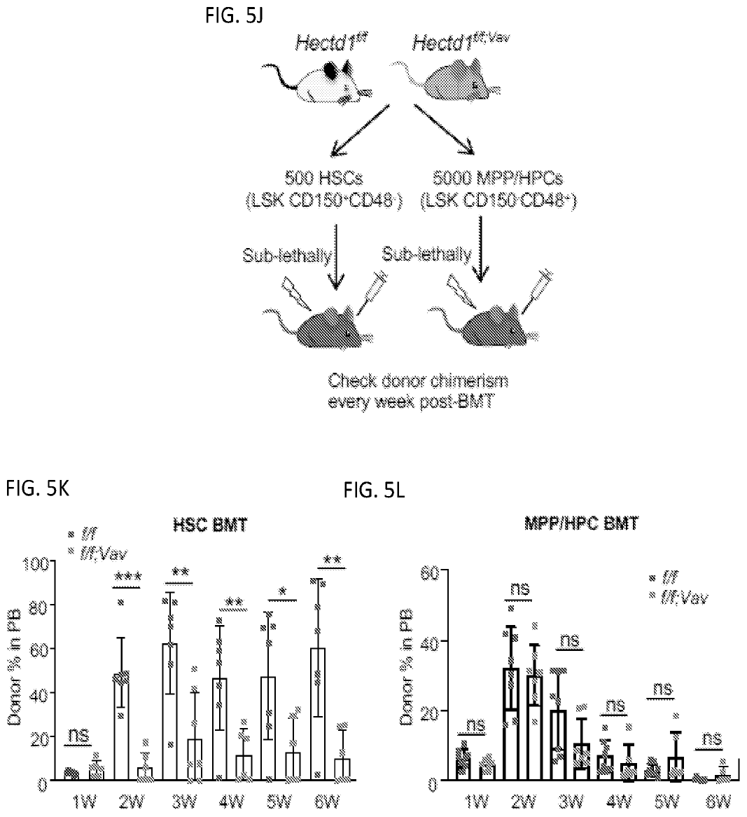

To further address if the decreased repopulation observed in total BM transplantation is due to intrinsic HSC properties, we purified LT-HSCs (LSK CD150⁺CD48⁻) from Hectd1^{f/f} and Hectd1^{f/f;Vav} mice by flow cytometric sorting and transplanted 100 HSCs into each lethally-irradiated recipient along with Sca1-depleted BM competitors (FIG. 5A) (Balcerek et al., 2018). Our results demonstrated that Hectd1-deficient HSCs exhibited inferior reconstitution ability to that of control HSCs in vivo (FIG. 5B). Importantly, donor-derived HSPCs in the BM of recipient mice transplanted with Hectd1-deficient HSCs were significantly reduced when compared with those from control HSCs (FIG. 5C), indicating Hectd1 insufficiency decreases intrinsic HSC activity.

We next analyzed the influence of HectD1 on the maintenance of HSC function upon ex vivo culture. Purified HSCs were cultured in media containing a combination of cytokines SCF, TPO, FLT3L and IL6 for 12 days. The resultant cultured cells from 100 HSCs were transplanted (FIG. 5A). Our data revealed that Hectd1 insufficiency failed to maintain HSCs ex vivo, which was accompanied by a marked reduction in reconstituting capacity (9.0% cultured HSCs vs 57.8% fresh HSCs reconstitution at 16 weeks), while control HSCs showed comparable reconstitution after culture (92.0% cultured HSCs vs 82.0% fresh HSCs) (FIG. 5D). Moreover, examination of donor-derived HSPCs in the BM of recipient mice revealed that Hectd1-deficient HSCs completely lost their stem cell identity in culture (FIG. 5E). Importantly, we found that Hectd1-deficient HSCs had a visibly slower growth rate than control HSCs ex vivo as determined by enumeration of cell numbers, and this phenotype persisted irrespective of cytokines used in culture (FIGS. 5F-5I). Taken together, our data suggest that HectD1 is critical for HSC function in vivo and HSC growth ex vivo.

Since HectD1 is widely expressed in a range of hematopoietic cells, we asked if HectD1 is preferentially required for HSC function. To test this, we injected 500 purified HSCs (LSK CD150⁺CD48⁻) or 5000 MPPs/hematopoietic progenitors (HPCs) (LSK CD150⁻CD48⁺) (Oguro et al., 2013; Pietras et al., 2015; Wilson et al., 2008) from Hectd1^{f/f} and Hectd1^{f/f;Vav} mice into each sub-lethally irradiated recipient and analyzed donor chimerism every week post-BMT (FIG. 5J). Of note, our results showed that Hectd1 deficiency markedly decreased HSC reconstitution ability, but not that of MPP/HPCs (FIGS. 5K, 5L and 4L, 4M), indicating that HectD1 plays a critical role in regulating HSC function.

HectD1 Interacts with, Ubiquitinates and Regulates the Stability of ZNF622

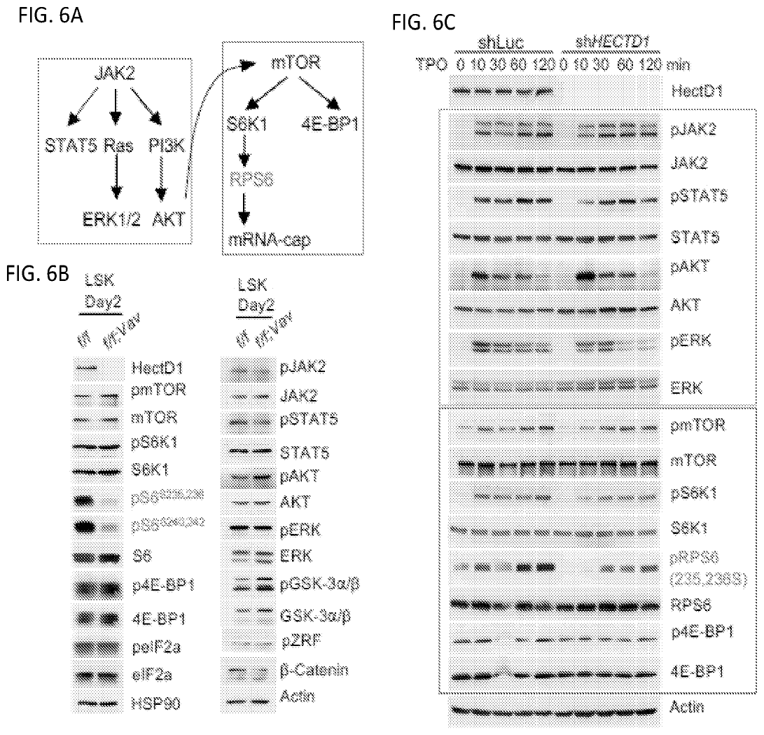

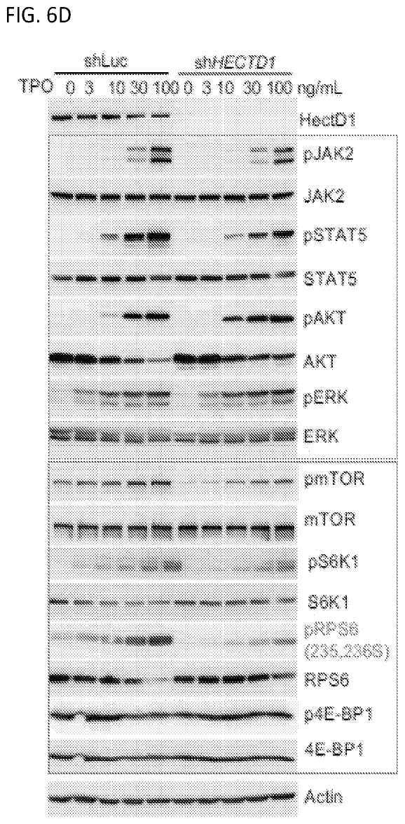

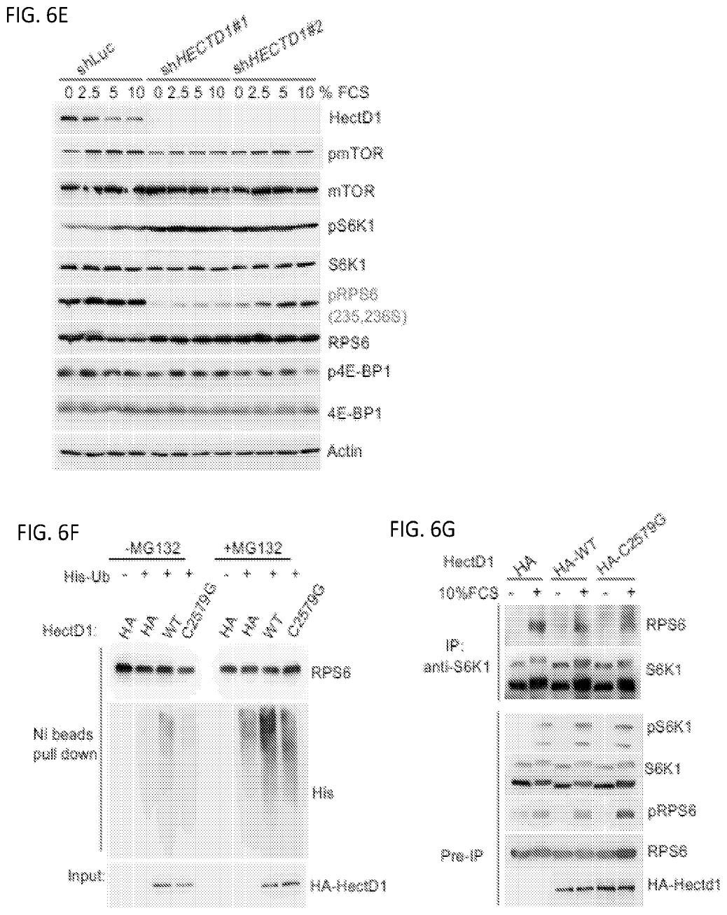

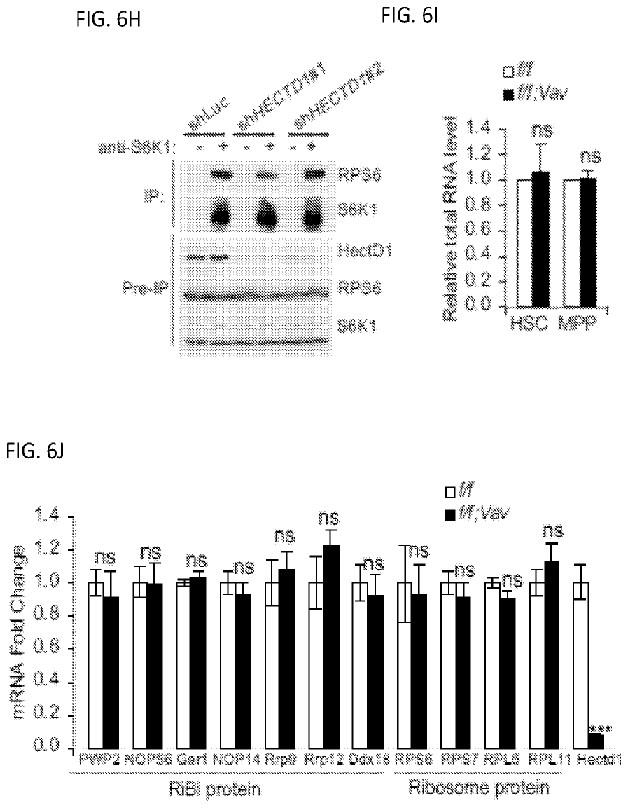

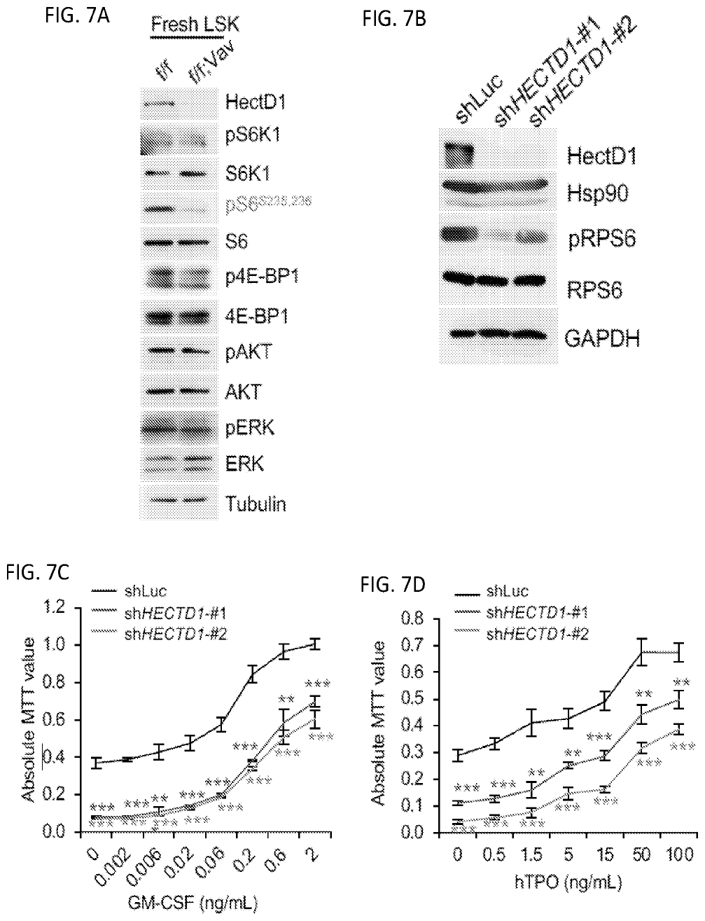

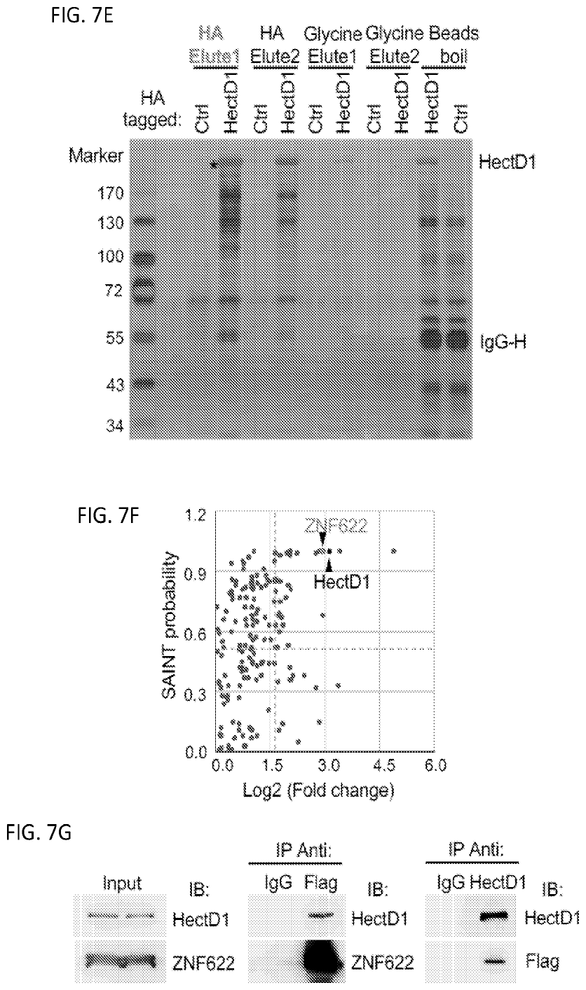

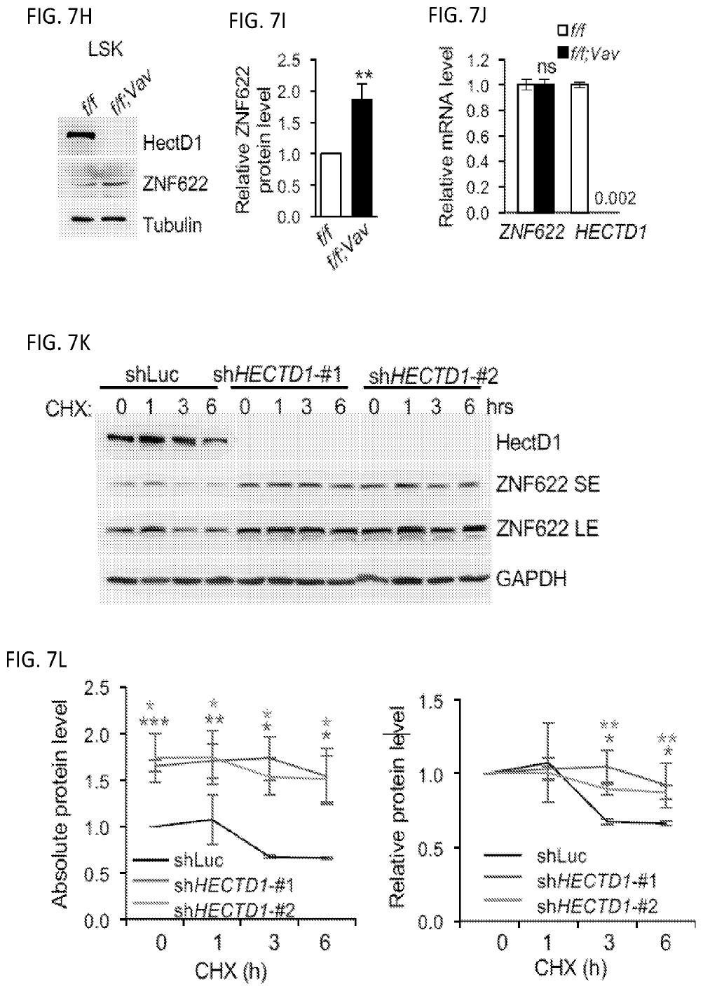

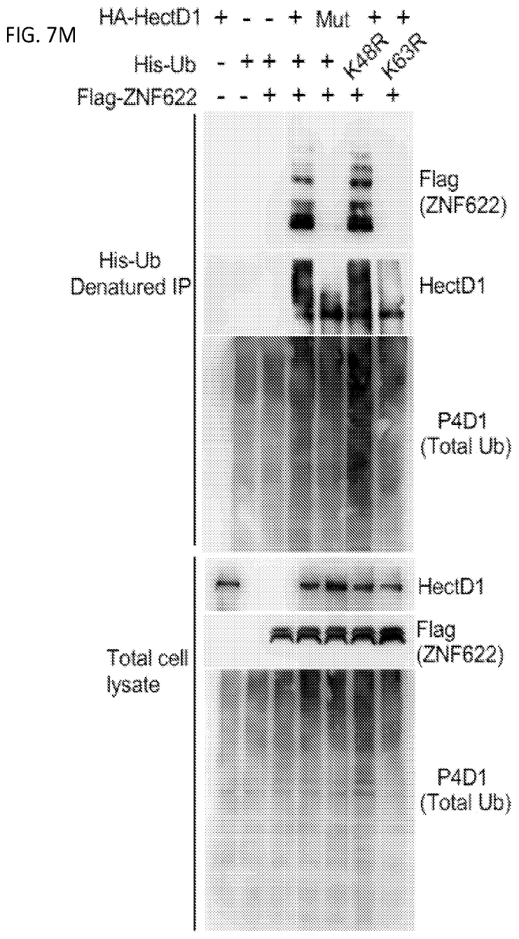

To study the mechanisms underlying the reduced growth of Hectd1-deficient HSPCs, we first evaluated known signaling pathways important for HSC function, ie, JAK-STAT, PI3K-AKT, MAPK-ERK, mTOR, GSK3 and β-catenin pathways (FIG. 6A). However, none of these signaling molecules were altered, except for a significant reduction of phospho-RPS6 (pRPS6) in both freshly isolated and cultured Hectd1-deficient LSK cells (FIGS. 7A and 6B).

To facilitate downstream biochemical studies, we resorted to cytokine-dependent human progenitor cell line, TF-1 cells. Using two different shRNAs to stably deplete HECTD1 in human TF-1 cells, in comparison to Luciferase (Luc) control (FIG. 7B), we observed a significant growth retardation in the presence of TPO or GM-CSF upon HECTD1 knockdown (FIGS. 7C and 7D), consistent with findings from HSPCs (FIGS. 5F-5I). TF-1 cells also recapitulated the signaling defects observed in primary LSK cells, which is a reduction in RPS6 phosphorylation but not any other pathways we examined (FIGS. 6C-6E). Thus, TF-1 cells appear to be a reliable and robust cell system for us to further dissect HectD1 functions.

Intriguingly, the reduction of pRPS6 seemed to be independent of mTOR pathway since p-mTOR, p4E-BPJ, pS6K1 and a S6K1 substrate pZRF (Barilari et al., 2017) were not changed (FIGS. 7A and 6B). This led us to hypothesize that HectD1 might directly ubiquitinate RPS6 or regulate S6K1-RPS6 interaction as K63-ubiquitination is known to affect protein complex formation (Shembade and Harhaj, 2015). However, neither RPS6 ubiquitination nor the S6K1-RPS6 interaction was impacted by HectD1 E3 dead mutant C2579G (Mut) (Sarkar and Zohn, 2012) or HECTD1 knockdown (FIGS. 6F-6H), implying that HectD1 indirectly affects RPS6 phosphorylation. The phospho-RPS6 has been shown to control the RiBi- (ribosome biogenesis) gene transcriptional program (Chauvin et al., 2014). We found that Hectd1-deficient HSCs did not exhibit changes in either total RNA level that is predominantly rRNAs or the mRNA level of the RiBi genes relatively to that of control cells (FIGS. 61 and 6J).

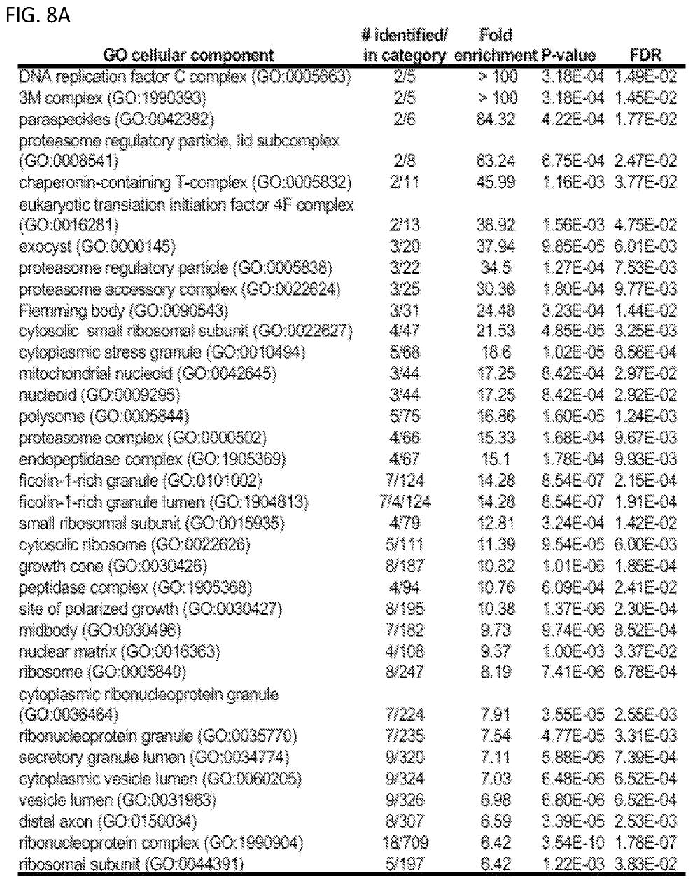

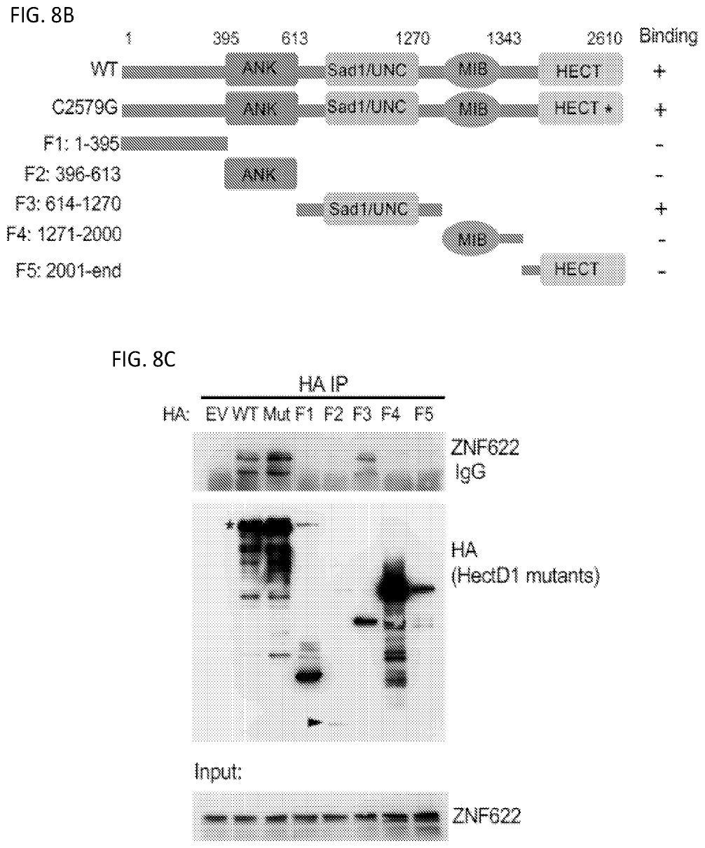

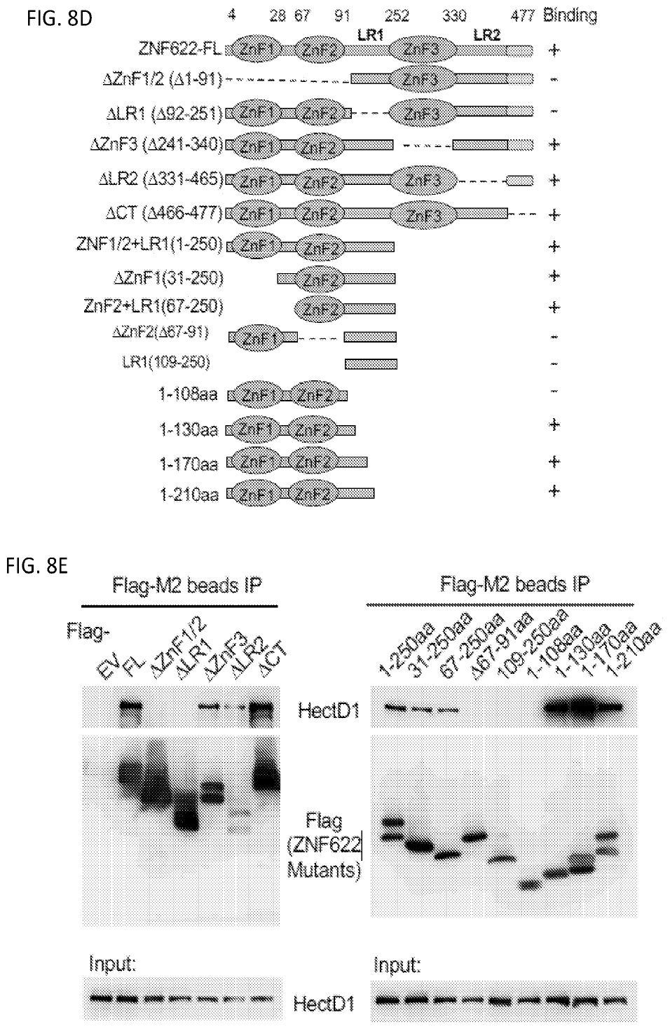

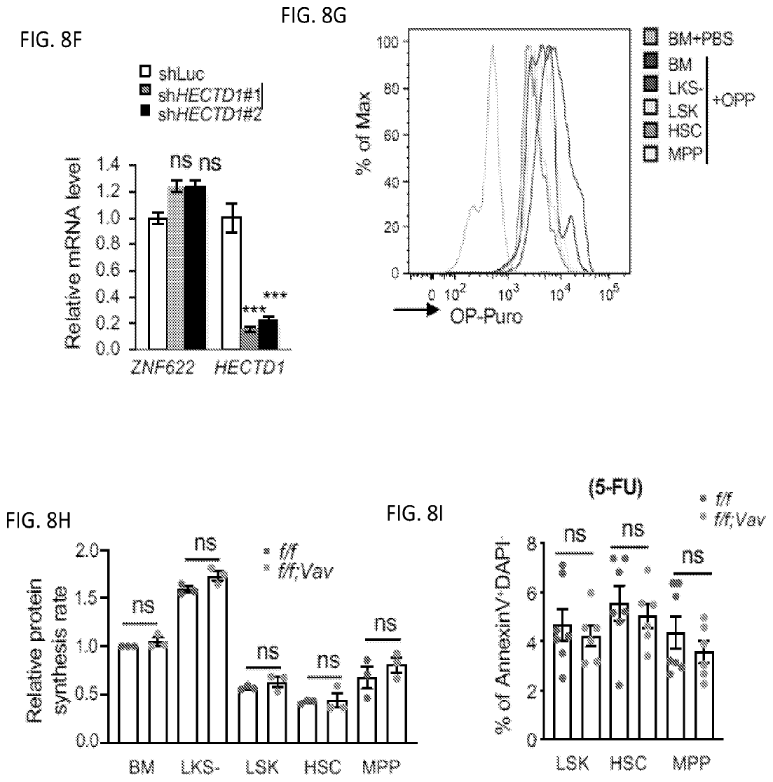

In an attempt to explore the molecular mechanism underlying the important role for Hectd1 in HSCs in a comprehensive and unbiased manner, we set out to identify HectD1 substrates by affinity purification of HectD1 interacting protein complex using mass spectrometry (MS). We transfected HA-tagged HectD1 or vector alone into HEK293T cells. HectdD1-interacting proteins were immunoprecipitated (IP) using anti-HA agarose beads, followed by HA peptide affinity elution. Glycine elution after HA elution detected very few bound proteins, and the non-specific proteins remained associated with the agarose beads upon boiling in SDS loading buffer, indicating the specificity and robustness of HA-affinity purification (FIG. 7E). We thus subjected the HA eluates to MS analysis along with vector control. Triplicates of IP-MS were performed and results were evaluated using the CRAPome (Contaminant Repository for Affinity Purification) analysis (Mellacheruvu et al., 2013). We ranked and selected the potential interacting proteins with the cutoff of SAINT (Significance Analysis of INTeractome) score ≥0.5 and fold change (FC) ≥3 (FIG. 7F). Gene ontology (GO) analysis revealed a high enrichment of ribosome/translation-related proteins and proteasome proteins (FIG. 8A), which is in agreement with the role of HectD1 as an E3 ubiquitin ligase and regulator of cell growth. Additionally, it suggested a potential role for HectD1 in ribosomes and protein synthesis.

Among the top hits, we focused on ZNF622 protein, given the critical role of its yeast homolog Rei1 in ribosome biogenesis (Greber et al., 2016; Meyer et al., 2010). The interaction between HectD1 and ZNF622 was first confirmed in Flag-ZNF622 reconstituted TF-1 cells by co-IP (FIG. 7G). Next, we generated a series of deletion mutants of HectD1 and ZNF622, and transfected them into 293T cells, followed by IP/WB analysis to map the responsible domain(s) for their interaction. Our data showed that Hectd1 interacted with ZNF622 regardless of its E3 activity as both HA-Hectd1 WT, and E3 dead mutant C2579G (Mut), but not vector control could pull down endogenous ZNF622. We also found that the Sad1/UNC domain of HectD1 is responsible for its interaction with ZNF622 (FIGS. 8B and 8C). Furthermore, deletion of either ZnF2 domain or the first linker region (LR1) located in the N-terminus of ZNF622 completely abolished its interaction with HectD1 (FIGS. 8D and 8E). Interestingly, cryo-EM analysis has revealed that that these two regions of Rei1 are located outside of the polypeptide tunnel (Greber et al., 2016; Kargas et al., 2019), rendering it accessible to HectD1 interaction.

Next, we asked if HectD1 regulates ZNF622 ubiquitination and protein stability. We found that Hectd1 deficiency increased ZNF622 protein, but not mRNA levels in LSK cells (FIGS. 7H-7J). Consistently, ZNF622 protein but not mRNA level was increased in HECTD1-depleted TF-1 cells (FIGS. 7K, 7L and 8F). Importantly, the half-life of ZNF622 proteins was significantly prolonged in HECTD1-depleted TF-1 cells in the presence of cycloheximide (CHX) that blocks nascent protein synthesis (FIGS. 7K and 7L). Next, we analyzed ZNF622 ubiquitination status impacted by HectD1 using Nickel-beads (Ni-NTA) pulldown of His-tagged Ub under denatured conditions to capture direct Ub-conjugation in ZNF622 proteins while reducing the detection of its associated proteins. Our data demonstrated that ZNF622 was robustly ubiquitinated by WT HectD1, but not the C2579G mutant. Moreover, consistent with the notion of HectD1 being an E3 ligase for lysine⁶³-polyubiquitination (K63-Ub) (Sarkar and Zohn, 2012), K63R, but not K48R Ub mutant, abolished ZNF622 ubiquitination (FIG. 7M), further demonstrating that ZNF622 is a direct substrate of HectD1. Taken together, our data suggest that HectD1 interacts with, ubiquitinates and regulates the stability of ZNF622.

### Hectd1 Insufficiency Decreases HSPC Proliferation and Global Translation Rates Upon Stress

The HectD1 IP-MS data and the interaction between HectD1-ZNF622 promoted us to investigate a potential role for HectD1 in protein synthesis and ribosome biology. To test this, we performed in vivo OP-puro (0-propargyl Puromycin) assay in primary adult Hectd1^{f/f} and Hectd1 ^{f/f;Vav} mice to evaluate protein synthesis rate (Liu et al., 2012). We found that HSCs exhibited a lower protein synthesis rate than restricted progenitors and mature hematopoietic cells (FIGS. 8G and 8H), consistent with the previous report (Signer et al., 2014). However, Hectd1 insufficiency did not affect protein synthesis rates in any of the hematopoietic cell subsets at the steady state (FIG. 8H).

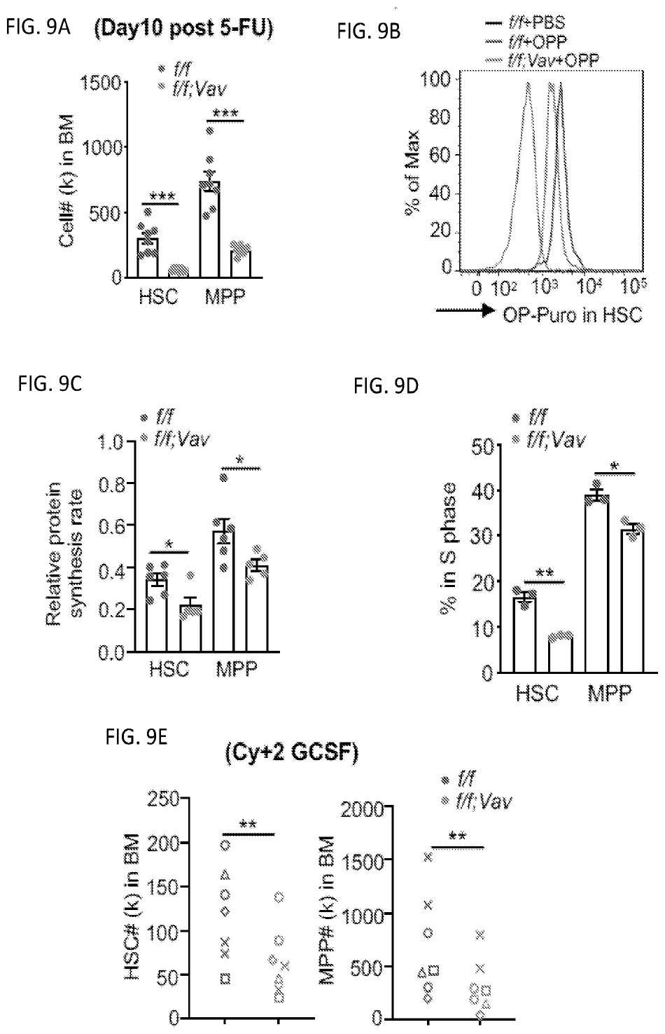

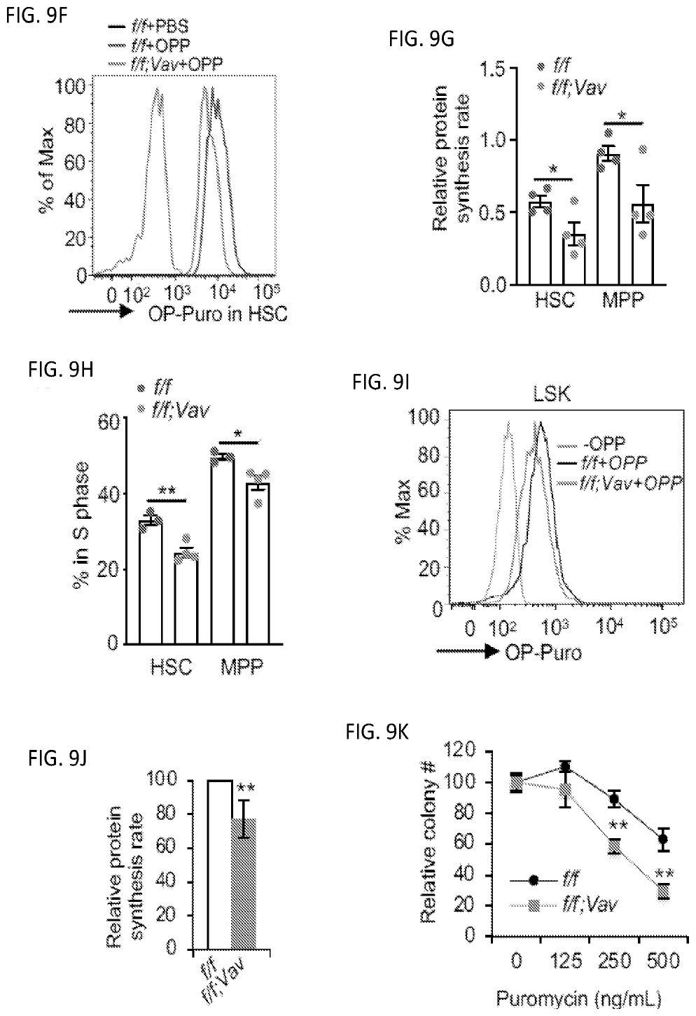

Our data showed that Hectd1 deficiency does not affect phenotypic HSC number in the steady state (FIG. 2G), but dramatically decreases functional HSCs in the transplantation assay or ex vivo culture, both of which conditions force HSCs to proliferate (FIGS. 3 and 5). Thus, we investigated if HectD1 is critical for HSC proliferation in vivo under stress conditions. We subjected the mice to two different types of stress. We first challenged Hectd1^{f/f} and Hectd1^{f/f;Vav} mice with cytoablative drug 5-fluorouracil (5-FU), which depletes cycling hematopoietic cells and drives primitive HSCs to regenerate. At day 10 after 5-FU administration, Hectd1^{f/f;Vav} mice exhibited a pronounced reduction in BM HSC and MPP numbers in comparison to the control mice (FIG. 9A). Notably, both Hectd1 deficient HSCs and MPPs showed reduced protein synthesis rate using the OP-Puro assay (FIGS. 9B and 9C), which correlated with their slower cycling status than the controls as determined by BrdU incorporation (FIG. 9D). We did not observe elevated cell death under 5-FU stress (FIG. 8I). To consolidate the conclusion of HectD1 function under stress conditions, we subjected mice to cyclophosphamide treatment followed by two daily doses of GCSF to induce HSPC proliferation (Morrison et al., 1997). In agreement, our data revealed lower HSC and MPP numbers (FIG. 9E), slower cell cycle kinetics, as well as decreased protein synthesis rate in Hectd1^{f/f;Vav} mice in comparison to the controls (FIGS. 9F-9H).

Next, we examined OP-Puro incorporation in ex vivo cultured LSK cells, which were also undergoing proliferative stress. Hectd1-deficient cells displayed a decrease in global translation rate by ≥20% when compared to control LSKs (FIGS. 91 and 9J). Consistently, Hectd1-deficient BM progenitors were hypersensitive to puromycin, an inhibitor of translation elongation (FIG. 9K). Of note, this observed translation defect in both primary LSK cells and TF-1 cells was independent of eIF2a or 4E-BP1 phosphorylation, two key regulators of translation initiation (FIGS. 6B-6E) (Holcik and Sonenberg, 2005). Taken together, we demonstrate that HectD1 plays a critical role in HSPC proliferation and protein synthesis under hematopoietic stress.

### Hectd1 Deficiency Disrupts ZNF622-Mediated 60S Ribosome Maturation and 60S/40S Subunit Joining

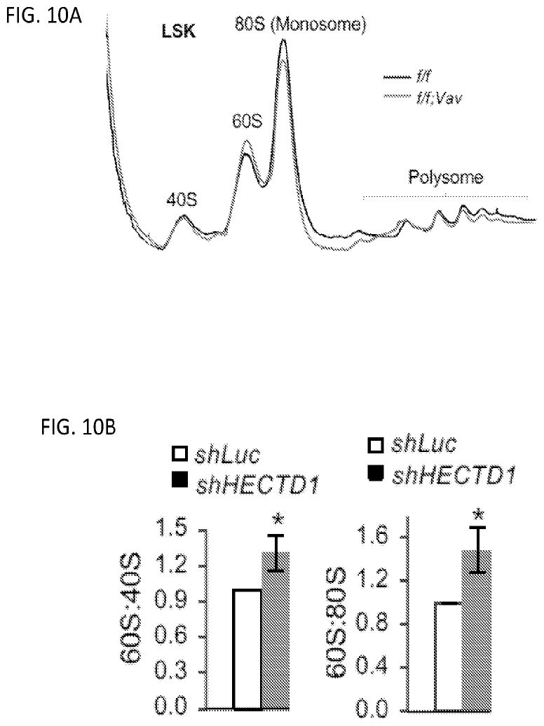

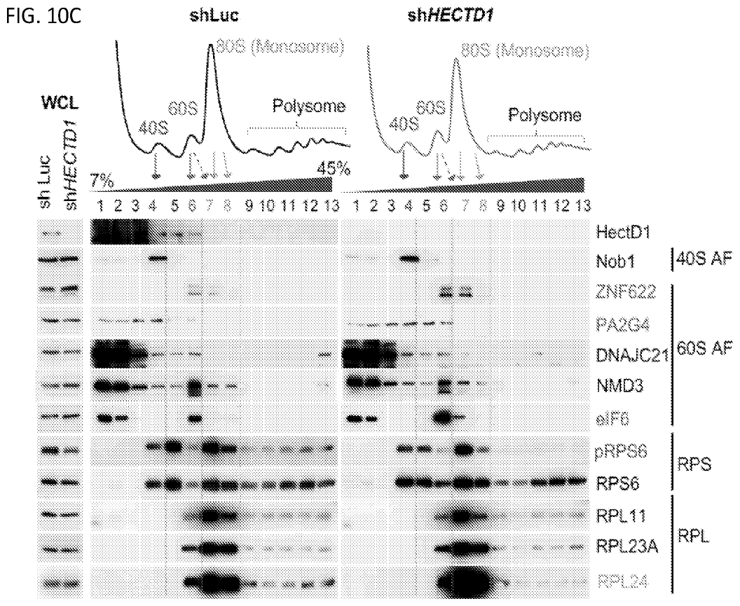

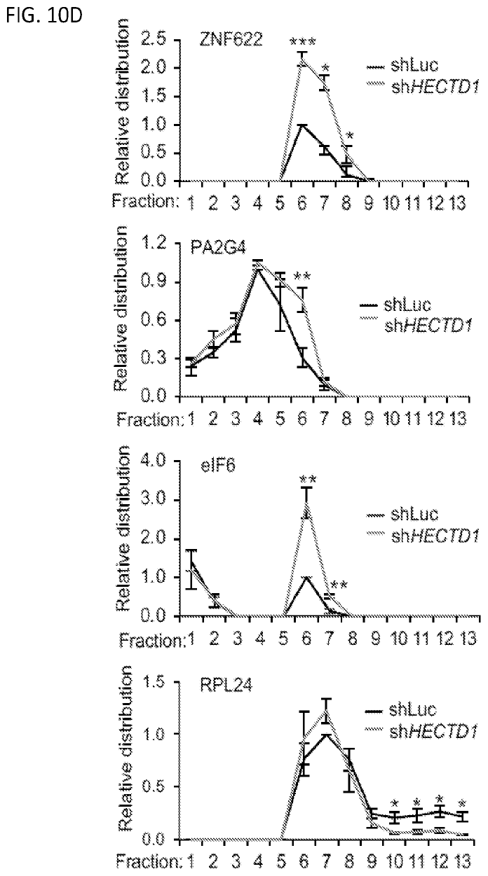

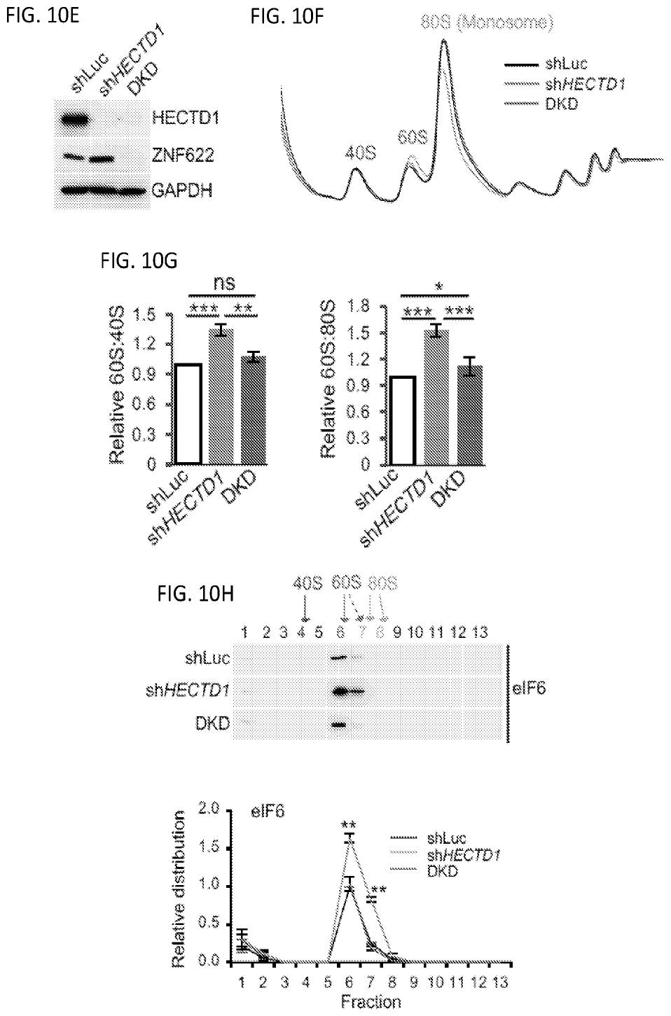

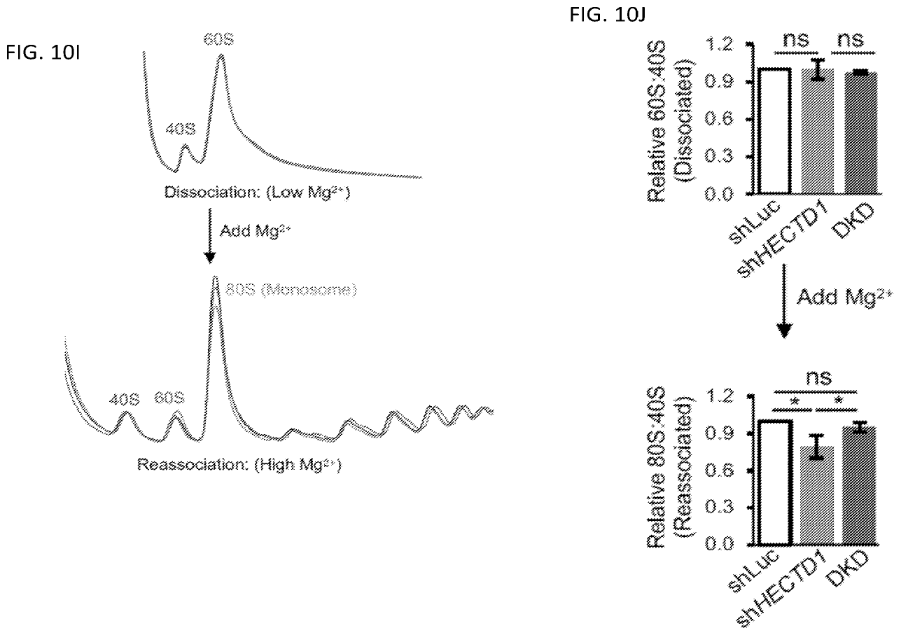

To dissect molecular mechanisms by which HectD1 regulates ribosome assembly and protein translation, we examined whether HectD1 affects ribosome composition by polysome profiling assay. Cell lysates of equal RNA content were fractionated in a sucrose gradient, followed by recording of the ultraviolet (UV) absorbance of each fraction. Hectd1-null LSK cells displayed an increased 60S content but decreased 80S monosome and polysome, while 40S remained unchanged (FIG. 10A). This phenotype was also observed in HECTD1-depleted TF-1 cells (FIG. 10B). These data indicated a previously-unrecognized role for HectD1 in regulating ribosome assembly and proper global translation.

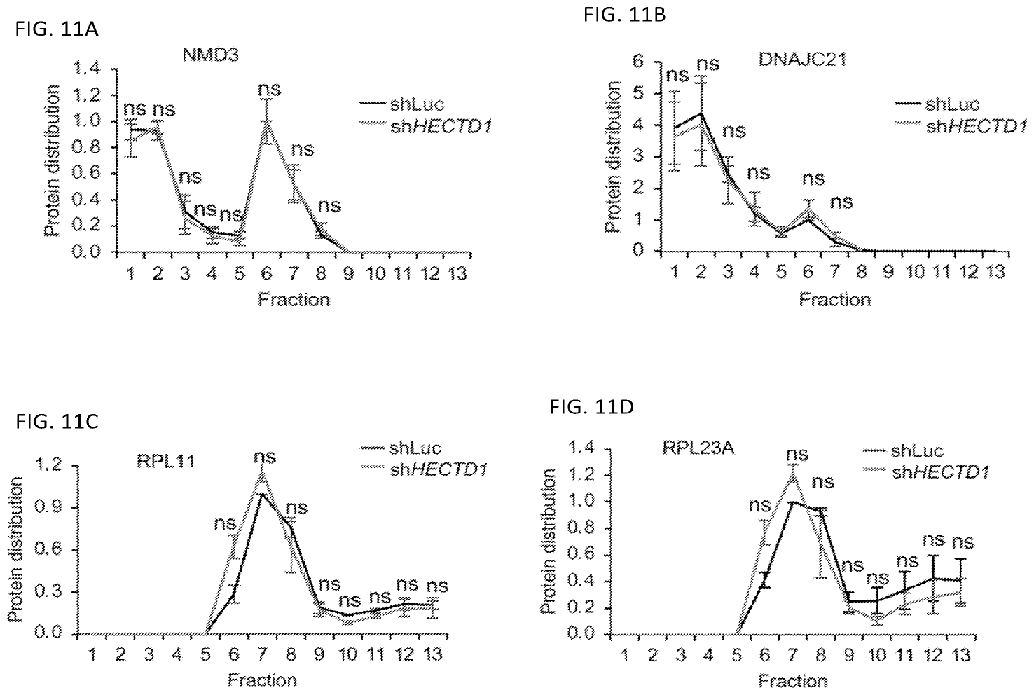

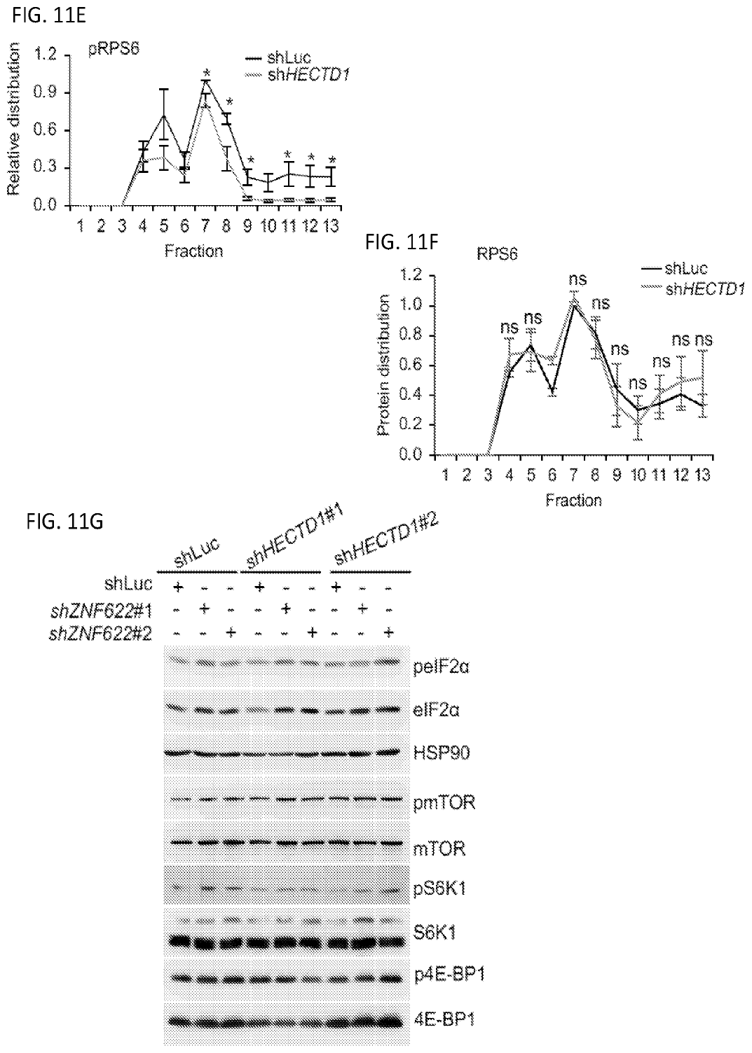

During ribosome biogenesis, subunits are exported to the cytoplasm as pre-60S and pre-40S complexes that must undergo final maturation involving sequential addition and release of a series of proteins to form mature 60S and 40S subunits (de la Cruz et al., 2015; Kargas et al., 2019). In yeast, ZNF622 homolog Rei1, stabilizes Arx1 (human PA2G4) association with pre-60S (Greber et al., 2012). Jjj1 (human DNAJC21) is required for the release of Rei1-Arx1 complex and the completion of polypeptide exit tunnel (PET) maturation (Lo et al., 2010; Meyer et al., 2010). Failure of Rei1/Arx1 dissociation from pre-60S particles precludes downstream assembly events, including the anti-association factor eIF6 release from pre-60S, which is a crucial step for 40S joining to form a functional ribosome. Thus, we sought to test if the elevated ZNF622 level in HECTD1-depleted cells would affect 60S maturation. Fractions of shLuc or shHECTD1 TF-1 cells from the sucrose gradient were collected and examined by WB. The results showed that HECTD1 was predominantly located in the cytosol, with a small fraction detected in free ribosome subunits (FIG. 10C). Notably, ZNF622 was predominantly associated with the 60S, and HECTD1 depletion resulted in a marked accumulation of ZNF622 in the 60S (FIGS. 10C and 10D). In contrast, the 40S protein Nob1 remained unchanged. Moreover, the ZNF622 binding partner PA2G4 and downstream eIF6 were markedly increased in the 60S, indicating an abnormal 60S ribosome formation (FIGS. 10C and 10D). Interestingly, the ribosome biogenesis factor for the maturation of the polypeptidyl transferase center (PTC), NMD3, was unchanged (FIGS. 10C and 11), indicating that the regulation of PET and PTC may be uncoupled. Besides the association and release of assembly factors, the cytoplasmic maturation of pre-60S subunit involves the concomitant incorporation of cytoplasmic ribosome proteins (de la Cruz et al., 2015; Kargas et al., 2019). Consistent with this notion, we found impaired incorporation of cytoplasmic RPL24 in the polysomes of HECTD1-depleted cells (FIGS. 10C and 10D), whereas other core RPL proteins that were pre-assembled in the nucleus, such as RPL11 and RPL23A, were unaffected (FIGS. 10C and 11). Of note, we observed a reduced pRPS6 level but not total RPS6 in HECTD1 deficient cells when compared to the controls (FIGS. 10C and 11). Together, these data suggest that increased ZNF622 proteins upon HECTD1 loss leads to a defective 60S maturation or activation of a translational quality control pathway arising from a block in the release of PA2G4 and eIF6, reduced joining of 60S and 40S, thereby resulting in reduced 80S and polysomes.

To gain further insight into the role for HectD1 in the regulation of ZNF622 in 60S formation and 60S/40S subunit joining, we assessed if depletion of ZNF622 could rescue the ribosome defects observed upon HECTD1 loss. We compared the polysome profiling and eIF6 retention in the 60S of shLuc control, HECTD1 single and HECTD1; ZNF622 double knockdown (DKD) TF-1 cells (FIG. 5E). Our data showed that knockdown of ZNF622 in HECTD1-deficient cells restored ribosome composition as well as eIF6 release to normal levels (FIGS. 10F-10H). Furthermore, we performed ribosome dissociation/reassociation assay with these cell lines to directly interrogate 60S/40S subunit joining (Burwick et al., 2012). 80S monosomes and polysomes were first dissociated into 40S and 60S subunits under low Mg²⁺ conditions, and the total amount of 40S and 60S was comparable among these three cell lines (FIGS. 101 and 10J). We subsequently added back Mg² allow 40S and 60S subunits to reassociate. HECTD1 depletion reduced ribosomal reassociation. Importantly, this disruption in ribosomal reassociation was reversed by ZNF622 knockdown (FIGS. 101 and 10J). Taken together, these results provide direct evidence that HectD1 plays an essential role in controlling 60S/40S subunit joining and translational control by regulating ZNF622.

### ZNF622 Depletion Restores Protein Translation and HSC Transplantation Activity Induced by Hectd1 Loss

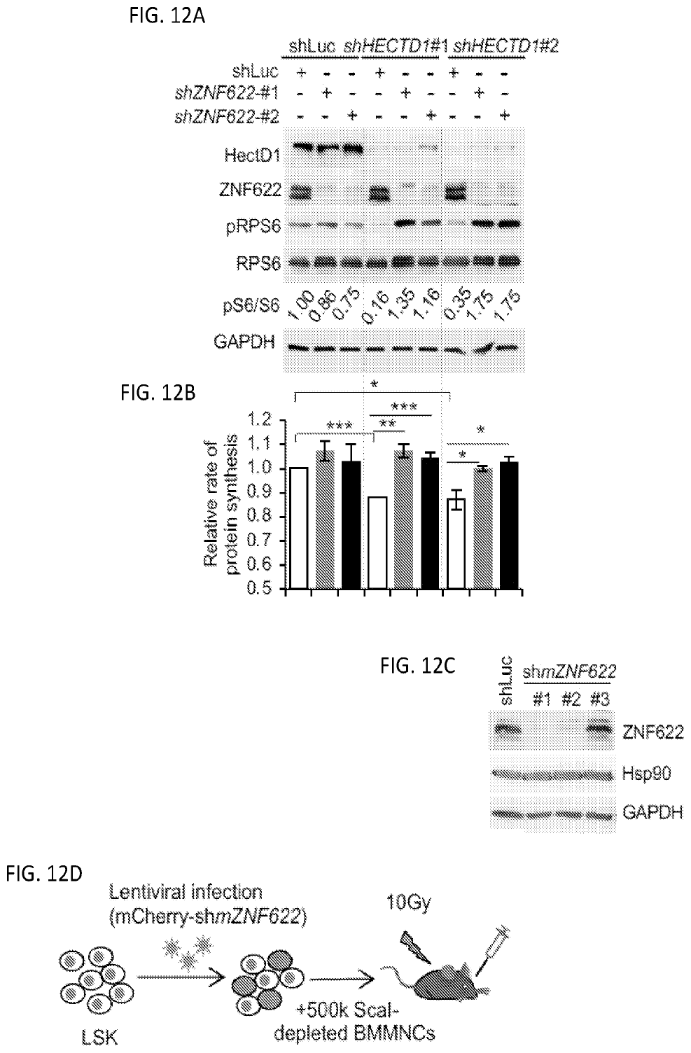

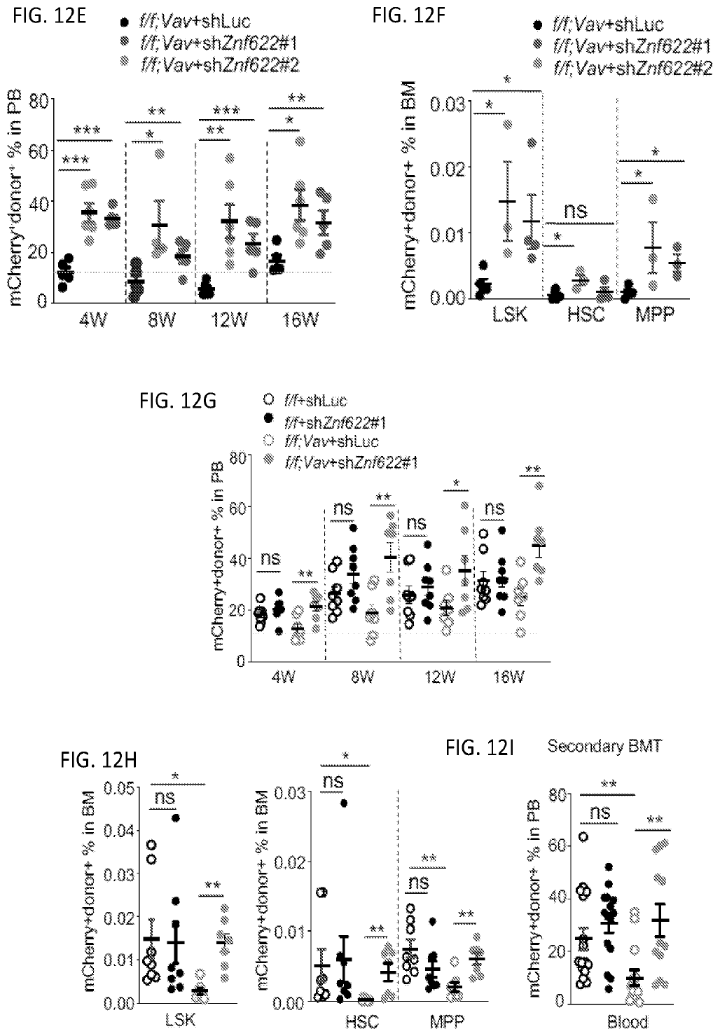

We reasoned that if the compromised translation rate observed in Hectd1-deficient cells was owing to the elevated ZNF622 level and ZNF622-mediated ribosome defects, attenuation of ZNF622 expression would be able to rescue this phenotype. To test this hypothesis, we knocked down ZNF622 with shRNA in Luc- or HECTD1-depleted TF-1 cells (FIG. 12A). Strikingly, ZNF622-depletion fully restored protein synthesis rate to normal levels in HECTD1-depleted cells, while it did not significantly affect the translation rate in control cells as examined by the OP-Puro assay (FIG. 12B). Of note, ZNF622 downregulation restored pRPS6 level in HECTD1-depleted cells that was independent of mTOR (FIGS. 12A and 11G).

We next asked if the aberrant accumulation of ZNF622 accounts for the perturbed HSC activity owing to Hectd1 deficiency. To functionally test this, we depleted Znf622 in Hectd1-null HSCs and examined their reconstitution ability. We first generated two efficient shRNAs against mouse Znf622 (shZnf622 #1 and #2) with #1 shRNA being the most robust (FIG. 12C). Subsequently, LSK cells were purified from Hectd1^{f/f;Vav} mice and infected with lentivirus expressing shLuc or two shRNAs to Znf622 with mCherry as a fluorescent marker. We injected 250k lentivirally-infected LSKs into each recipient with 500k Sca1-depleted competitor BM cells to ensure high donor reconstitution and recipient survival (FIG. 12D) (Jiang et al., 2012). Both Znf622 shRNAs increased the reconstitution of Hectd1-deficient HSCs in the peripheral blood (PB) of the recipient mice (FIG. 12E). Importantly, mice transplanted with LSKs depleted of Znf622 had a significantly higher proportion of mCherry⁺ cells in the BM HSCs and MPPs than those with shLuc (FIG. 12F). Therefore, the restored donor chimerism observed in the PB was resulted from HSC restoration.

We next examined if the rescue effect of Znf622 downregulation was specific to Hectd1-null background. LSKs from both Hectd1^{f/f} and Hectd1^{f/f;Vav} mice were sorted and infected with lentivirus expressing shLuc or shZnf622 #1 followed by BMT. While control HSC reconstitution in the PB was not significantly affected by Znf622 knockdown, Hectd1-deficient HSC reconstitution ability was significantly rescued (FIG. 12G), and importantly, that was attributed to restored HSPC populations (FIG. 12H). Strikingly, mCherry⁺ donor chimerism in the PB of secondary recipients further validated the HSC promoting effect of Znf622 knockdown as the reconstitution ability of Znf622-depleted Hectd1-null cells was significantly elevated in comparison to control cells (FIG. 12I). To summarize, these data support a working model that ZNF622 is an important functional mediator of HectD1 function in regulating ribosome assembly and protein translation efficiency, as well as HSC regeneration.

### DISCUSSION

Tightly-regulated protein synthesis rate is critical for HSC maintenance and function, as only a 30% decrease (using Rp124^{Bst/+} mice, where ribosome protein Rp124 is partially depleted) or increase (cKO of Pten or 4E-BP1/2 mice) in protein synthesis is sufficient to impair HSC proliferation and self-renewal (Signer et al., 2014; Signer et al., 2016). Here we identified a critical role for ubiquitin-dependent regulation of ribosome assembly by HectD1 to meet the increased protein demands during HSC regeneration in vivo and ex vivo.

We demonstrate that HectD1 is required for HSC but not progenitor cell expansion in vivo, pointing to that balanced protein synthesis is essential for HSC function. Interestingly, HectD1 is dispensable for HSC development during homeostasis, but is critical for HSC regeneration under proliferative stress. Of note, HectD1 is found indispensable in all hematopoietic stress conditions we tested, such as in vivo transplant settings, ex vivo expansion under cytokines, as well as genotoxic stress 5-FU or cyclophosphamide/GCSF-induced HSPC proliferation. The extraordinary demands for HSPC growth and proliferation in these conditions require increased global protein production, thereby coordinated ribosome production. The data in this report suggest that HectD1 controls ribosome assembly and protein synthesis rate during HSC regeneration by regulating 60S assembly factor ZNF622. Of note, we cannot exclude the possibility that HectD1 also regulates protein synthesis and cell cycle in some, and potentially many, hematopoietic progenitors after injury.

Impaired ribosome function arising from genetic aberrations of RP proteins or assembly factors causes a group of human disorders known as “ribosomopathies”, such as SDS. 90% of SDS patients harbor mutations in the SBDS gene, which functions with the GTPase EFL1 to facilitate the removal of anti-association factor eIF6 from pre-60S ribosomal subunits to allow the assembly of 40S and 60S into functional monosomes (Finch et al., 2011; Menne et al., 2007; Warren, 2018; Weis et al., 2015). In addition, biallelic mutations in DNAJC21, a maturation factor for the PET, cause abnormal accumulation of PA2G4 and eIF6 in pre-60S ribosomal subunits and reduce 60S and 40S joining, eliciting an SDS-like phenotype (Dhanraj et al., 2017; Tummala et al., 2016). It is noteworthy that DNAJC21 assists the release of ZNF622-PA2G4 from pre-60S, allowing for the progression of downstream maturation steps. Thus, our data suggest that Hectd1 deficiency recapitulates both the molecular ribosomal abnormalities and the phenotypic perturbations in HSCs, reminiscent of SDS. We found that depletion of ZNF622 in HECTD1-deficient cells rescues ribosome composition as well as eIF6 release to normal levels, thereby restoring 60S/40S subunit joining and protein translation. Notably, it also supports the idea that ZNF622 influences the affinity of eIF6 for the ribosome, thereby serving as a quality control step to ensure proper ribosome assembly. More importantly, we demonstrate that downregulation of Znf622 rescues HSC reconstitution capacity in Hectd1-null mice, implicating ZNF622 inhibition as a potential therapeutic strategy for the treatment of BMF disease with defective ribosomes.

It is important to point out that structure-function studies of ribosome biogenesis and assembly factors have been most examined in yeast and prokaryotes. Our work provides significant biochemical and functional insight into the critical biogenesis factors of human and mouse ribosomes. Structural analysis of the yeast homolog of ZNF622, Rei1, revealed that the Rei1 C-terminus is deeply inserted into the 60S PET, which is essential for the proofreading of PET maturation and subsequent Arx1 (yeast PA2G4) liberation and eIF6 eviction steps (Greber et al., 2016). Our data in human hematopoietic cells demonstrate that accumulated ZNF622 in pre-60S blocks PA2G4 removal and efficient ribosome maturation, in accordance with the function of Rei1 in Arx1 release in yeast cells (Meyer et al., 2010). An alternative but not mutually-exclusive explanation points to a subunit joining and translation defect rather than a biogenesis defect upon HECTD1 loss, since the amount of total 40S and 60S was unaffected. In the absence of HECTD1, ZNF622 abnormally accumulates in the pre-60S and/or rebinds to mature 60S to block 60S/40S joining via ZNF622-associated eIF6. A role for ZNF622 and eIF6 in ribosomal stress and translational quality control has been suggested in a whole-genome CRISPRi screen in human chronic myeloid leukemia (CML) cell line. This study revealed that depletion of ZNF622 or eIF6 restores cell fitness and cell growth in the presence of PF8503, a translational inhibitor that binds to the PET and inhibits the translation of selective proteins and suppresses cell proliferation (Liaud et al., 2019). Hence, our work may uncover a ribosome quality control pathway that is critical for HSPCs during regenerative or proliferative stress. Under this circumstance, high demand for protein synthesis increases the need for ribosome quality control, where HECTD1 activity controls ZNF622 and eIF6-mediated ribosome assembly and protein translation efficiency.

In yeast, Rei1-Arx1 departure from 60S coincides with the exchange for the Rei1 family member Reh1 in the PET that persists in the later stages of cytoplasmic maturation process (Kargas et al., 2019; Ma et al., 2017). This finding is in striking contrast to those reported in yeast, in which dual knockout of Rei1 and Reh1 severely constrains yeast cell growth (Greber et al., 2016). Therefore, it is possible that ZNF622 exerts a distinct function from its yeast homologs. The N-terminus of Rei1 contacts RPL24 on the surface of the 60S ribosome (Greber et al., 2016) and directly interacts with eIF6 (Kargas et al., 2019). ZNF622 may regulate eIF6 release through its direct interaction with eIF6, or indirectly through RPL24. In agreement, we demonstrate that increased ZNF622 protein level coincides with an accumulation of eIF6 in the 60S subunit of HECTD1-depleted cells and depletion of ZNF622 reduces eIF6 association in the 60S. Of note, RPL24 is essential for the formation of 60S-40S inter-subunit bridges, one of which depends on the direct interaction between RPL24 and RPS6 (Kisly et al., 2019). Our data raise the possibility that RPS6 phosphorylation serves as a feedback regulation in the 40S subunit when the 60S large subunit encounters stress, as we observed a correlation between pRPS6 and 60S maturation and protein synthesis in this context. Nonetheless, the intricate regulation between different ribosome subunits and monosomes/polysomes mandates future investigations.

Taken together, we reveal a previously unappreciated ubiquitin dependent control mechanism for ribosome assembly and protein synthesis that is essential to HSC activity and regeneration, highlighting the importance of ribosome assembly factors in HSC function. Importantly, we demonstrate that Znf622 depletion restores 60S ribosome maturation, ribosome assembly, protein synthesis and HSC activity in the context of Hectd1 deficiency. This finding establishes an in vivo example of genetic suppression of HSC defects associated with dysfunctional ribosomes and provides new avenues for treatment strategies for ribosomopathies.

### Example II

### Methods for Downmodulating ZNF622 Activity for the Treatment of Bone Marrow Failure

Inhibition of ZNF622 Ameliorates HSC Functional Defects Associated with SDS

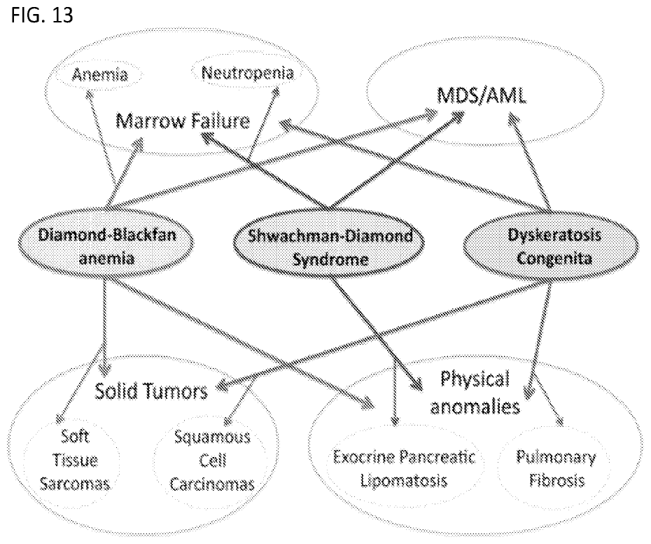

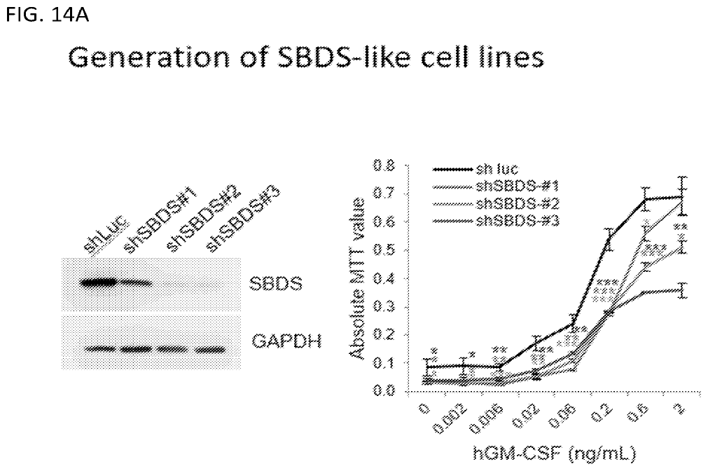

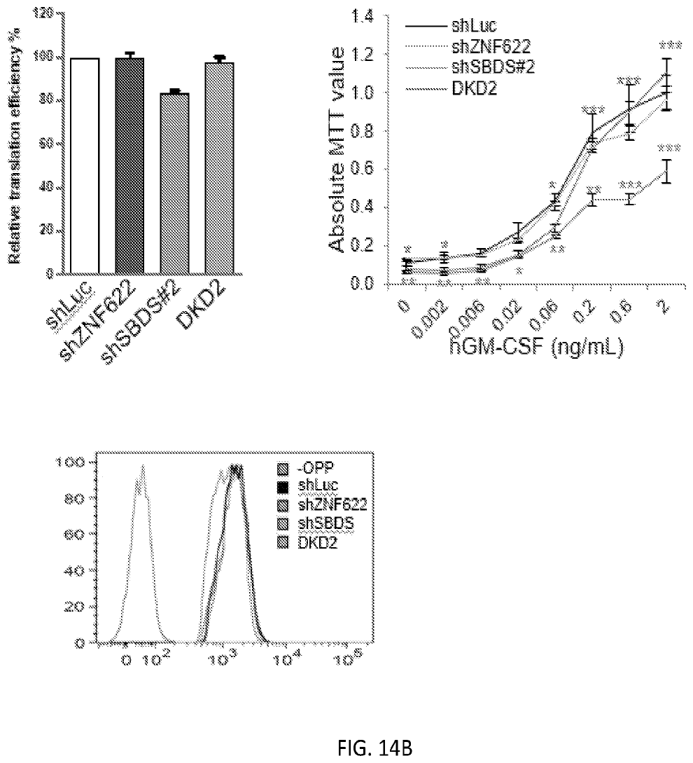

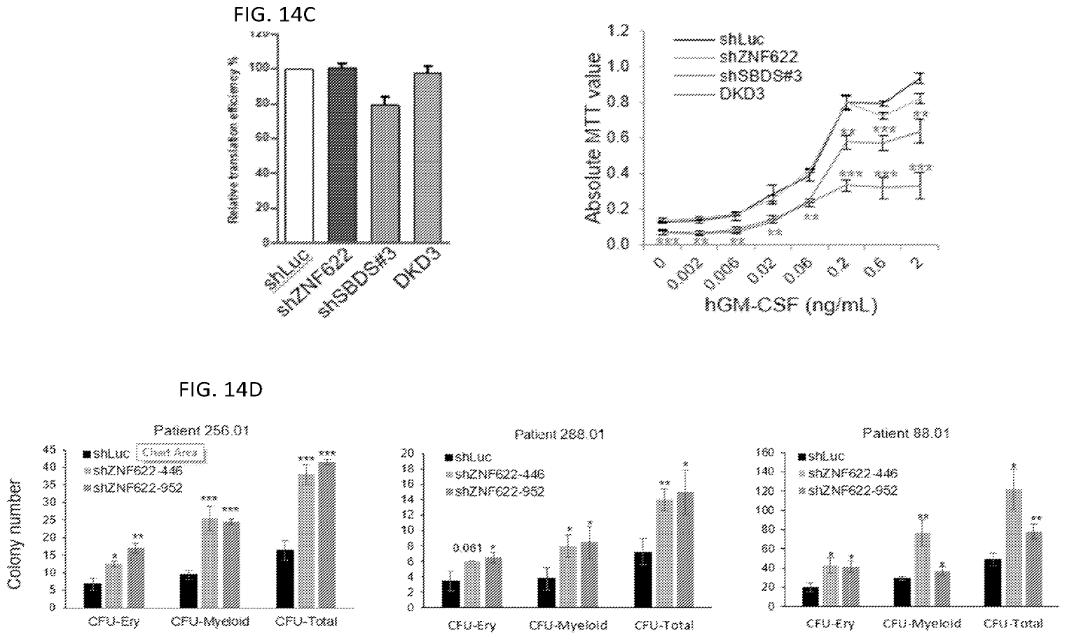

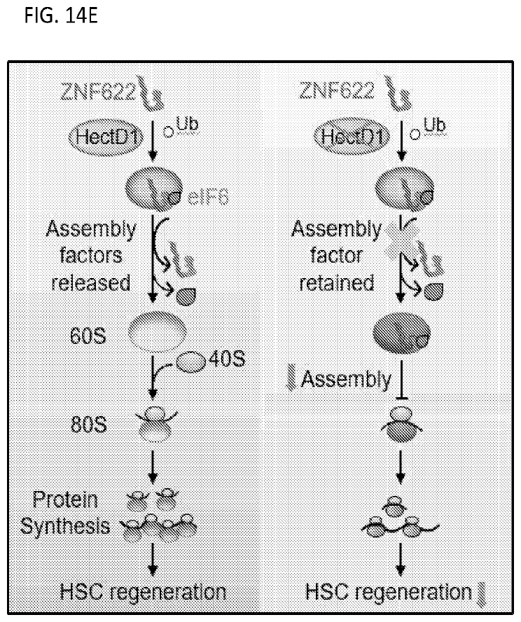

In view of the data presented in Example I, showing that Znf622 depletion restores cell growth and HSC function associated with ribosome defects, we next tested ZNF622 depletion could restore human SDS cells associated with SBDS mutations (FIG. 13). We first tested if ZNF622 downregulation could restore protein synthesis and cell growth of SBDS-depleted cells in shLuc, shZNF622, shSBDS or ZNF622; SBDS dual-depleted cells. The data showed that while knockdown of SBDS decreased protein synthesis and cell proliferation, recapitulating human Shwachman-Diamond syndrome (SDS) patient phenotype. (FIG. 14A). Importantly, knockdown of ZNF622 restored both protein synthesis and cell growth in SBDS-depleted TF-1 cells (FIGS. 14B and 14C).

### ZNF622 Depletion Improves the Function of SDS Patient-Derived HSPCs

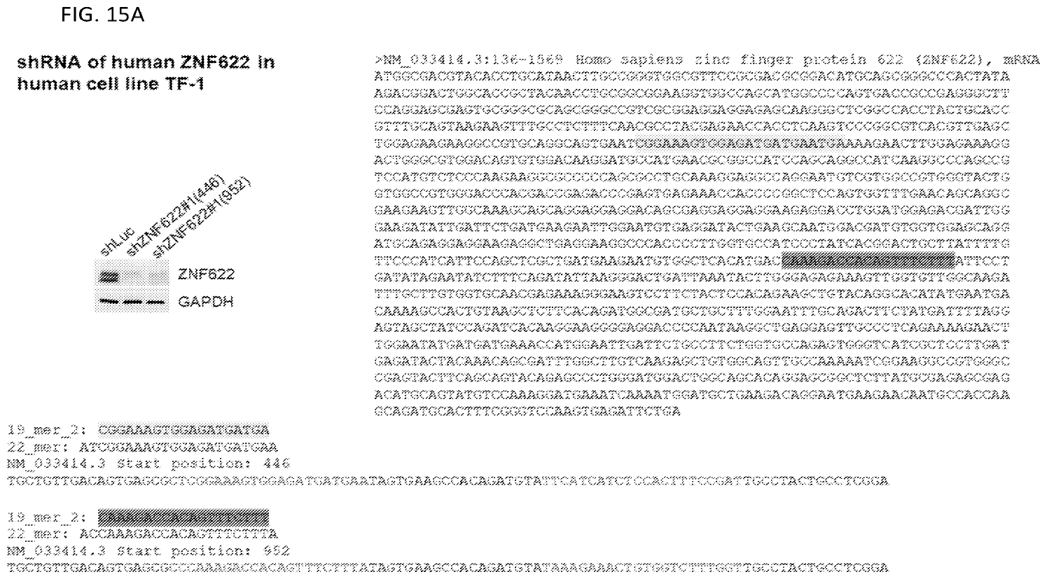

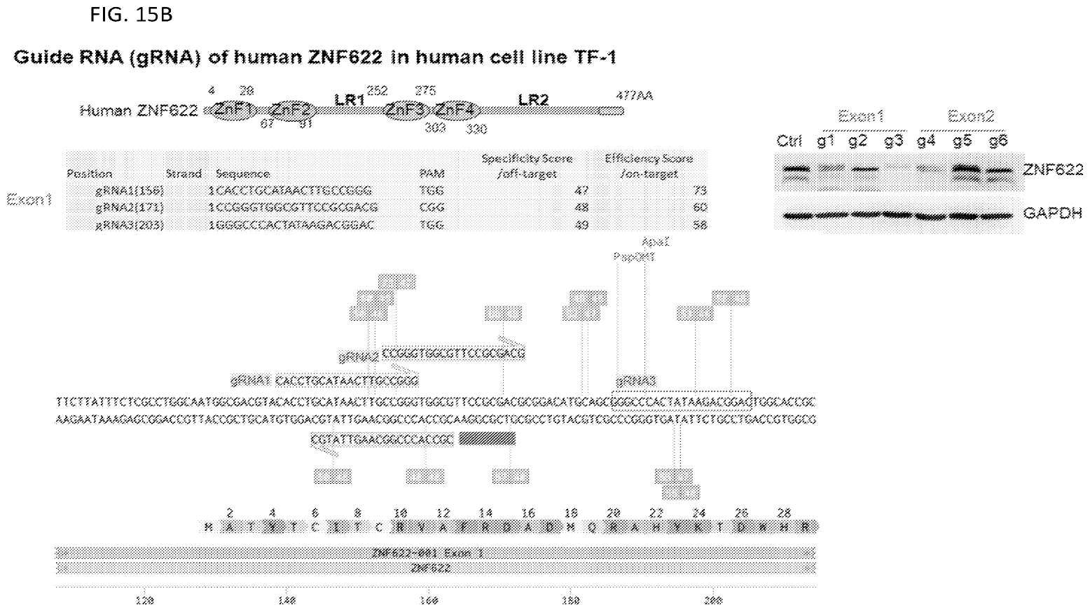

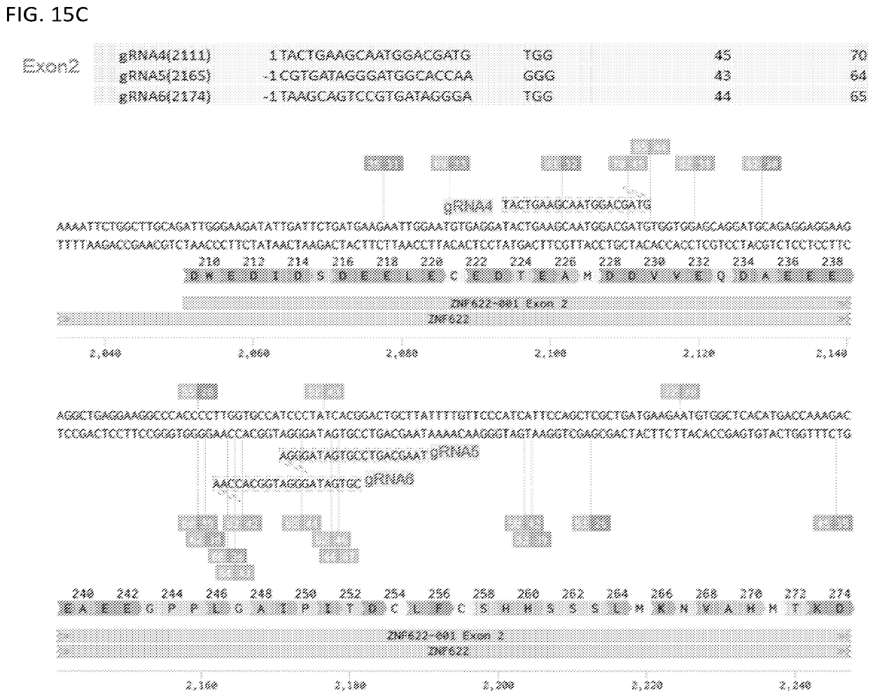

Next, in view of the promising results obtained with murine HSPC transplant data, we tested the potential of ZNF622 inhibition in primary BM CD34⁺HSPCs isolated from three SDS patients. We showed that two different miR30-based shRNAs to ZNF622 exhibited high efficiency in depleting ZNF622 proteins (FIG. 15A). We purified SDS patient HSPCs transduced with lentiviral shRNAs targeting ZNF622 or Luc by flow cytometric sorting, then performed CFC (colony forming cell) assay (FIG. 14D). We found ZNF622 depletion increased CFU progenitor potentials of all three SDS patients with SBDS mutations (FIG. 14D). These data strongly indicate that ZNF622 is a promising new therapeutic target for treating Shwachman-Diamond syndrome (SDS) by improving HSPC functions via restoration of ribosome assembly (FIG. 14E).

These data indicate that our approach can be used to transplant gene modified patient-derived HSPCs into humanized mice by infusion. HSPCs from SDS patient can be transduced with lentiviral vectors containing shRNAs to ZNF622, or electroporated CRSPR-Cas containing gRNA to ZNF622 into NBSGW or NSG mice. 4-6 months post transplant, we will examine levels of human CD45⁺leukocyte chimerism in the peripheral blood, bone marrow, and spleen, by flow cytometry. We will assess if ZNF622 inhibition enhances SDS HSC engraftment in the BM and spleen compared to control. We will also evaluate hematopoietic lineage reconstitution, as well as HSPC expansion in xenotransplanted mice. Based on the murine data obtained to date, we suggest that ZNF622 depletion expands and improves the function of SDS HSPCs in vivo.

Generation of gRNA that Efficiently Target ZNF622 Depletion

Similarly, the powerful genome editing CRISPR/Cas tools we generated in FIG. 15 can be used to advantage to confirm the effects of ZNF622 deficiency in primary HSPCs SDS patients as described in FIG. 14 when shRNAs were employed. Using the CRISPR/Cas system, we will knockout ZNF622 in primary HSPCs from SDS patients, and plate them onto methylcellulose culture media to demonstrate that ZNF622 depletion restores the growth and colony-formation ability of SDS HSPCs. Furthermore, we can then infuse them into NSG or NBSGW mice to confirm that ZNF622 depletion restores the engraftment, expansion, and blood cell production of SDS HSCs in mice in vivo.

We have generated 6 different gRNAs for robust deletion of ZNF622 in cell lines, TF1 cells, via CRISPR/Cas9 system (FIGS. 15B and 15C). The sequence, location, as well as deletion efficiency are shown in FIGS. 15B and 15C. Specifically, we first generated TF1 cells stably expressing Cas9. Then we used lentivirus carrying guide RNA (gRNA) to ZNF622 to infect TF1/Cas9 cells. Two days later, we collected cell pellets from control gRNA and 6 gRNAs to ZNF622 for western blot analysis (FIGS. 15A and 15B). To rule out any incomplete knockout efficiency, we sorted single clones from control gRNA and ZNF622 gRNA expressing cells and confirmed the 100% knockout efficiency by sequencing and WB. These data enable the production of therapeutic tools useful for treatment of human autologous stem cells for reinfusion back into the patient after ex vivo expansion.

### REFERENCES

Aleidi, S. M., Yang, A., Sharpe, L. J., Rao, G., Cochran, B. J., Rye, K. A., Kockx, M., Brown, A. J., and Gelissen, I. C. (2018). The E3 ubiquitin ligase, HECTD1, is involved in ABCA1-mediated cholesterol export from macrophages. Biochim Biophys Acta Mol Cell Biol Lipids 1863, 359-368.
Balcerek, J., Jiang, J., Li, Y., Jiang, Q., Holdreith, N., Singh, B., Chandra, V., Lv, K., Ren, J. G., Rozenova, K., et al. (2018). Lnk/Sh2b3 deficiency restores hematopoietic stem cell function and genome integrity in Fancd2 deficient Fanconi anemia. Nat Commun 9, 3915.
Barilari, M., Bonfils, G., Treins, C., Koka, V., De Villeneuve, D., Fabrega, S., and Pende, M. (2017). ZRF1 is a novel S6 kinase substrate that drives the senescence programme. EMBO J 36, 736-750.
Bersenev, A., Wu, C., Balcerek, J., and Tong, W. (2008). Lnk controls mouse hematopoietic stem cell self-renewal and quiescence through direct interactions with JAK2. J Clin Invest 118, 2832-2844.
Boocock, G. R., Morrison, J. A., Popovic, M., Richards, N., Ellis, L., Durie, P. R., and Rommens, J. M. (2003). Mutations in SBDS are associated with Shwachman-Diamond syndrome. Nat Genet 33, 97-101.
Burwick, N., Coats, S. A., Nakamura, T., and Shimamura, A. (2012). Impaired ribosomal subunit association in Shwachman-Diamond syndrome. Blood 120, 5143-5152.
Chauvin, C., Koka, V., Nouschi, A., Mieulet, V., Hoareau-Aveilla, C., Dreazen, A., Cagnard, N., Carpentier, W., Kiss, T., Meyuhas, O., et al. (2014). Ribosomal protein S6 kinase activity controls the ribosome biogenesis transcriptional program. Oncogene 33, 474-483.
Chen, M. J., Yokomizo, T., Zeigler, B. M., Dzierzak, E., and Speck, N. A. (2009). Runxl is required for the endothelial to haematopoietic cell transition but not thereafter. Nature 457, 887-891.
Choi, H., Liu, G., Mellacheruvu, D., Tyers, M., Gingras, A. C., and Nesvizhskii, A. I. (2012). Analyzing protein-protein interactions from affinity purification-mass spectrometry data with SAINT. Curr Protoc Bioinformatics Chapter 8, Unit8 15.
D′Alonzo, D., Emch, F. H., Shen, X., Bruder, E., De Geyter, C., and Zhang, H. (2019). Hectd1 is essential for embryogenesis in mice. Gene Expr Patterns 34, 119064.
de la Cruz, J., Karbstein, K., and Woolford, J. L., Jr. (2015). Functions of ribosomal proteins in assembly of eukaryotic ribosomes in vivo. Annu Rev Biochem 84, 93-129.
Dhanraj, S., Matveev, A., Li, H., Lauhasurayotin, S., Jardine, L., Cada, M., Zlateska, B., Tailor, C. S., Zhou, J., Mendoza-Londono, R., et al. (2017). Biallelic mutations in DNAJC21 cause Shwachman-Diamond syndrome. Blood 129, 1557-1562.
Duhamel, S., Goyette, M. A., Thibault, M. P., Filion, D., Gaboury, L., and Cote, J. F. (2018). The E3 Ubiquitin Ligase HectD1 Suppresses EMT and Metastasis by Targeting the +TIP ACF7 for Degradation. Cell Rep 22, 1016-1030.
Eng, J. K., McCormack, A. L., and Yates, J. R. (1994). An approach to correlate tandem mass spectral data of peptides with amino acid sequences in a protein database. J Am Soc Mass Spectrom 5, 976-989.
Finch, A. J., Hilcenko, C., Basse, N., Drynan, L. F., Goyenechea, B., Menne, T. F., Gonzalez Fernandez, A., Simpson, P., D'Santos, C. S., Arends, M. J., et al. (2011). Uncoupling of GTP hydrolysis from eIF6 release on the ribosome causes Shwachman-Diamond syndrome. Genes Dev 25, 917-929.
Greber, B. J., Boehringer, D., Montellese, C., and Ban, N. (2012). Cryo-EM structures of Arx1 and maturation factors Rei1 and Jjj 1 bound to the 60S ribosomal subunit. Nat Struct Mol Biol 19, 1228˜1233.
Greber, B. J., Gerhardy, S., Leitner, A., Leibundgut, M., Salem, M., Boehringer, D., Leulliot, N., Aebersold, R., Panse, V. G., and Ban, N. (2016). Insertion of the Biogenesis Factor Rei1 Probes the Ribosomal Tunnel during 60S Maturation. Cell 164, 91-102.
Holcik, M., and Sonenberg, N. (2005). Translational control in stress and apoptosis. Nat Rev Mol Cell Biol 6,318-327.
Hu, Y., and Smyth, G. K. (2009). ELDA: extreme limiting dilution analysis for comparing depleted and enriched populations in stem cell and other assays. J Immunol Methods 347, 70-78.
Jiang, J., Balcerek, J., Rozenova, K., Cheng, Y., Bersenev, A., Wu, C., Song, Y., and Tong, W. (2012). 14-3-3 regulates the LNK/JAK2 pathway in mouse hematopoietic stem and progenitor cells. J Clin Invest 122, 2079-2091.
Kargas, V., Castro-Hartmann, P., Escudero-Urquijo, N., Dent, K., Hilcenko, C., Sailer, C., Zisser, G., Marques-Carvalho, M. J., Pellegrino, S., Wawiorka, L., et al. (2019). Mechanism of completion of peptidyltransferase centre assembly in eukaryotes. Elife 8.
Kisly, I., Remme, J., and Tamm, T. (2019). Ribosomal protein eL24, involved in two intersubunit bridges, stimulates translation initiation and elongation. Nucleic Acids Res 47, 406-420.
Klinge, S., and Woolford, J. L., Jr. (2019). Ribosome assembly coming into focus. Nat Rev Mol Cell Biol 20, 116-131.
Li, W., Hu, Y., Oh, S., Ma, Q., Merkurjev, D., Song, X., Zhou, X., Liu, Z., Tanasa, B., He, X., et al. (2015). Condensin I and II Complexes License Full Estrogen Receptor alpha-Dependent Enhancer Activation. Mol Cell 59, 188-202.
Liaud, N., Horlbeck, M. A., Gilbert, L. A., Gjoni, K., Weissman, J. S., and Cate, J. H. D. (2019). Cellular response to small molecules that selectively stall protein synthesis by the ribosome. PLoS Genet 15, e1008057.
Liu, J., Xu, Y., Stoleru, D., and Salic, A. (2012). Imaging protein synthesis in cells and tissues with an alkyne analog of puromycin. Proc Nati Acad Sci USA 109, 413-418.
Lo, K. Y., Li, Z., Bussiere, C., Bresson, S., Marcotte, E. M., and Johnson, A. W. (2010). Defining the pathway of cytoplasmic maturation of the 60S ribosomal subunit. Mol Cell 39, 196-208.
Lv, K., Jiang, J., Donaghy, R., Riling, C. R., Cheng, Y., Chandra, V., Rozenova, K., An, W., Mohapatra, B. C., Goetz, B. T., et al. (2017). CBL family E3 ubiquitin ligases control JAK2 ubiquitination and stability in hematopoietic stem cells and myeloid malignancies. Genes Dev 31, 1007-1023.
Ma, C., Wu, S., Li, N., Chen, Y., Yan, K., Li, Z., Zheng, L., Lei, J., Woolford, J. L., Jr., and Gao, N. (2017). Structural snapshot of cytoplasmic pre-60S ribosomal particles bound by Nmd3, Lsg1, Tif6 and Reh1. Nat Struct Mol Biol 24, 214-220.
Mellacheruvu, D., Wright, Z., Couzens, AL., Lambert, J. P., St-Denis, N. A., Li, T., Miteva, Y. V., Hauri, S., Sardiu, M. E., Low, T. Y., et al. (2013). The CRAPome: a contaminant repository for affinity purification-mass spectrometry data. Nat Methods 10, 730-736.
Menne, T. F., Goyenechea, B., Sanchez-Puig, N., Wong, C. C., Tonkin, L. M., Ancliff, P. J., Brost, R. L., Costanzo, M., Boone, C., and Warren, A. J. (2007). The Shwachman-Bodian-Diamond syndrome protein mediates translational activation of ribosomes in yeast. Nat Genet 39, 486-495.
Meyer, A. E., Hoover, L. A., and Craig, E. A. (2010). The cytosolic J-protein, Jjj1, and Rei1 function in the removal of the pre-60 S subunit factor Arx1. J Biol Chem 285, 961-968.
Mi, H., Muruganujan, A., Ebert, D., Huang, X., and Thomas, P. D. (2019). PANTHER version 14: more genomes, a new PANTHER GO-slim and improvements in enrichment analysis tools. Nucleic Acids Res 47, D419-D426.
Modlich, U., Schambach, A., Li, Z., and Schiedlmeier, B. (2009). Murine hematopoietic stem cell transduction using retroviral vectors. Methods Mol Biol 506, 23-31.
Morrison, S. J., Wright, D. E., and Weissman, I. L. (1997). Cyclophosphamide/granulocyte colony-stimulating factor induces hematopoietic stem cells to proliferate prior to mobilization. Proc Natl Acad Sci USA 94, 1908-1913.
Oguro, H., Ding, L., and Morrison, S. J. (2013). SLAM family markers resolve functionally distinct subpopulations of hematopoietic stem cells and multipotent progenitors. Cell Stem Cell 13, 102-116.
Peng, J., and Gygi, S. P. (2001). Proteomics: the move to mixtures. J Mass Spectrom 36, 1083-1091.
Pietras, E. M., Reynaud, D., Kang, Y. A., Carlin, D., Calero-Nieto, F. J., Leavitt, A. D., Stuart, J. M., Gottgens, B., and Passegue, E. (2015). Functionally Distinct Subsets of Lineage-Biased Multipotent Progenitors Control Blood Production in Normal and Regenerative Conditions. Cell Stem Cell 17, 35-46.
Rodriguez, C. I., Buchholz, F., Galloway, J., Sequerra, R., Kasper, J., Ayala, R., Stewart, A. F., and Dymecki, S. M. (2000). High-efficiency deleter mice show that FLPe is an alternative to Cre-loxP. Nat Genet 25, 139-140.
Rozenova, K., Jiang, J., Donaghy, R., Aressy, B., Greenberg, R. A., and Tong, W. (2015). MERIT40 deficiency expands hematopoietic stem cell pools by regulating thrombopoietin receptor signaling. Blood 125, 1730-1738.
Ruggero, D., and Shimamura, A. (2014). Marrow failure: a window into ribosome biology. Blood 124, 2784-2792.
Sarkar, A. A., Nuwayhid, S. J., Maynard, T., Ghandchi, F., Hill, J. T., Lamantia, A. S., and Zohn, I. E. (2014). Hectd1 is required for development of the junctional zone of the placenta. Dev Biol 392, 368-380.
Sarkar, A. A., and Zohn, I. E. (2012). Hectd1 regulates intracellular localization and secretion of Hsp90 to control cellular behavior of the cranial mesenchyme. J Cell Biol 196, 789-800.
Shembade, N., and Harhaj, E. W. (2015). Elucidating dynamic protein-protein interactions and ubiquitination in NF-kappaB signaling pathways. Methods Mol Biol 1280, 283-295.
Shevchenko, A., Wilm, M., Vorm, O., and Mann, M. (1996). Mass spectrometric sequencing of proteins silver-stained polyacrylamide gels. Anal Chem 68, 850-858.
Signer, R. A., Magee, J. A., Salic, A., and Morrison, S. J. (2014). Haematopoietic stem cells require a highly regulated protein synthesis rate. Nature 509, 49-54.
Signer, R. A., Qi, L., Zhao, Z., Thompson, D., Sigova, A. A., Fan, Z. P., DeMartino, G. N., Young, R. A., Sonenberg, N., and Morrison, S. J. (2016). The rate of protein synthesis in hematopoietic stem cells is limited partly by 4E-BPs. Genes Dev 30, 1698-1703.
Stadtfeld, M., and Graf, T. (2005). Assessing the role of hematopoietic plasticity for endothelial and hepatocyte development by non-invasive lineage tracing. Development 132, 203-213.
Sugrue, K. F., Sarkar, A. A., Leatherbury, L., and Zohn, I. E. (2019). The ubiquitin ligase HECTD1 promotes retinoic acid signaling required for development of the aortic arch. Dis Model Mech 12.
Sulima, S. O., Hofman, I. J. F., De Keersmaecker, K., and Dinman, J. D. (2017). How Ribosomes Translate Cancer. Cancer Discov 7, 1069-1087.
Tan, S., Kermasson, L., Hoslin, A., Jaako, P., Faille, A., Acevedo-Arozena, A., Lengline, E., Ranta, D., Poiree, M., Fenneteau, O., et al. (2019). EFL1 mutations impair eIF6 release to cause Shwachman-Diamond syndrome. Blood 134, 277-290.
Tran, H., Bustos, D., Yeh, R., Rubinfeld, B., Lam, C., Shriver, S., Zilberleyb, I., Lee, M. W., Phu, L., Sarkar, A. A., et al. (2013). HectD1 E3 ligase modifies adenomatous polyposis coli (APC) with polyubiquitin to promote the APC-axin interaction. J Biol Chem 288, 3753-3767.
Tummala, H., Walne, A. J., Williams, M., Bockett, N., Collopy, L., Cardoso, S., Ellison, A., Wynn, R., Leblanc, T., Fitzgibbon, J., et al. (2016). DNAJC21 Mutations Link a Cancer-Prone Bone Marrow Failure Syndrome to Corruption in 60S Ribosome Subunit Maturation. Am J Hum Genet 99, 115-124.
Warren, A. J. (2018). Molecular basis of the human ribosomopathy Shwachman-Diamond syndrome. Advances in biological regulation 67, 109-127.
Weis, F., Giudice, E., Churcher, M., Jin, L., Hilcenko, C., Wong, C. C., Traynor, D., Kay, R. R., and Warren, A. J. (2015). Mechanism of eIF6 release from the nascent 60S ribosomal subunit. Nat Struct Mol Biol 22, 914-919.
Wilson, A., Laurenti, E., Oser, G., van der Wath, R. C., Blanco-Bose, W., Jaworski, M., Offner, S., Dunant, C. F., Eshkind, L., Bockamp, E., et al. (2008). Hematopoietic stem cells reversibly switch from dormancy to self-renewal during homeostasis and repair. Cell 135, 1118-1129.
Woloszynek, J. R., Rothbaum, R. J., Rawls, A. S., Minx, P. J., Wilson, R. K., Mason, P. J., Bessler, M., and Link, D. C. (2004). Mutations of the SBDS gene are present in most patients with Shwachman-Diamond syndrome. Blood 104, 3588-3590.
Wong, C. C., Traynor, D., Basse, N., Kay, R. R., and Warren, A. J. (2011). Defective ribosome assembly in Shwachman-Diamond syndrome. Blood 118, 4305-4312.
Zohn, I. E., Anderson, K. V., and Niswander, L. (2007). The Hectd1 ubiquitin ligase is required for development of the head mesenchyme and neural tube closure. Dev Biol 306, 208-221.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of lowering ZNF622 levels in a human stem cell (HSC) and improving HSC reconstitution ability in a patient in need thereof, the method comprising administration of a therapeutically effective amount of a composition comprising a synthetic nucleic acid molecule targeting ZNF622, wherein lowering ZNF622 levels in said cell increases ribosomal subunit joining thereby improving protein synthesis rates in a cell or tissue, wherein the synthetic nucleic acid molecule is selected from an shRNA, an siRNA, an antisense oligonucleotide, and a guide strand suitable for CRISPR editing ZNF622 targeted nucleic acids.

2. A method of treating, delaying the onset of, ameliorating, and/or reducing a disease, disorder and/or condition, or a symptom thereof, associated with one or more ribosomopathy in a patient in need thereof, comprising administering to the patient a therapeutically effective amount of a synthetic nucleic acid targeting ZNF622, wherein the disease, disorder and/or condition, or the symptom thereof, associated with altered ribosomal subunit joining is treated, inhibited, the onset delayed, ameliorated, and/or reduced in the patient, wherein the synthetic nucleic acid molecule is selected from an shRNA, an siRNA, an antisense oligonucleotide, and a guide strand suitable for CRISPR editing.

3. The method of claim 1, wherein said synthetic nucleic acid targeting ZNF622 is modified to increase stability and/or uptake in vivo.

4. The method of claim 3, wherein said synthetic nucleic acid targeting ZNF622 is an shRNA.

5. The method of claim 1, wherein said synthetic nucleic acid encoding said shRNA is cloned into a vector.

6. The method of claim 3, wherein said vector is selected from a plasmid vector, a lentiviral vector, a retroviral vector, an AAV vector, and an adenovirus associated vector.

7. The method of claim 2, wherein the disease, disorder and/or condition is anemia.

8. The method of claim 7, wherein the anemia is hereditary anemia, myelodysplastic syndrome or severe chronic hemolysis.

9. The method of claim 7, wherein the anemia is associated with cancer.

10. The method of claim 8, wherein the hereditary anemia is sickle cell anemia, thalassemia, Fanconi anemia, Diamond Blackfan anemia, Shwachman Diamond syndrome, and red cell membrane disorders.

11. The method of claim 4, where said synthetic nucleic acid is an shRNA is cloned into a lentiviral vector and is shown in FIG. 15A, or wherein CRISPR editing of a ZNF622 encoding nucleic acid is employed using guide strands shown in FIGS. 15B and 15C.

12. (canceled)

13. The method of claim 11, wherein the nucleic acid is modified and has a nucleobase sequence that is at least 90%, at least 95%, at least 99%, or 100% complementary to all or a portion of a human ZNF622 nucleic acid.

14. The method of claim 13, wherein the modified oligonucleotide comprises at least one modified internucleoside linkage, at least one nucleoside of the modified oligonucleotide comprises a modified sugar or at least one nucleoside of the modified oligonucleotide comprises a modified nucleobase.

15. The method of claim 2, further comprising an additional active pharmaceutical agent useful for treatment of bone marrow failure.

16. A composition for reducing expression of ZNF622, comprising a synthetic nucleic acid molecule targeting, and specifically hybridizing to, a ZNF622 encoding nucleic acid, selected from an shRNA, an siRNA, an antisense oligonucleotide, and a guide strand suitable for CRISPR editing in a biologically acceptable carrier.

17. The composition of claim 16, wherein said synthetic nucleic acid is modified to increase stability in bodily fluids and/or uptake in a cell of interest and targets a ZNF622 contiguous nucleic acid shown in FIG. 7.

18. (canceled)

19. The composition of claim 16, wherein said synthetic nucleic acid molecule is present in a vector.

20. The composition according to claim 16, which is formulated for ex vivo cellular administration, parenteral administration, and intravenous administration.

21. Human stem cells comprising the composition of claim 20, said stem cells being suitable for transplantation into a recipient suffering from a ribosomopathy.

22. The composition of claim 21, wherein said stem cells are autologous stem cells or obtained from an immunologically compatible donor.

23. (canceled)

24. A method for treating a ribosomopathy, comprising administration of an effective amount of the transformed stem cells of claim 21 into a patient in need thereof.

25. The method of claim 2, where said synthetic nucleic acid is an shRNA is cloned into a lentiviral vector and is shown in FIG. 15A or CRISPR editing of a ZNF622 encoding nucleic acid is employed using guide strands shown in FIGS. 15B and 15C.


SEQUENCE LISTING (50 sequences)

SEQ ID NO: 1 - DNA, 20 bp, Artificial Sequence
  Hectd1 FRTt F
tgcctgctatcaagcctgat

SEQ ID NO: 2 - DNA, 24 bp, Artificial Sequence
  Common_En2 R
ccaactgaccttgggcaagaacat

SEQ ID NO: 3 - DNA, 20 bp, Artificial Sequence
  Hectd1 FRTt R
cagagagctggcaggaaagt

SEQ ID NO: 4 - DNA, 20 bp, Artificial Sequence
  Hectd1 ES loxp F
tccagcttggtatttgccca

SEQ ID NO: 5 - DNA, 20 bp, Artificial Sequence
  Hectd1 ES loxp R
actgatggcgagctcagacc

SEQ ID NO: 6 - DNA, 23 bp, Artificial Sequence
  Cre F
gcggtctggcagtaaaaactatc

SEQ ID NO: 7 - DNA, 23 bp, Artificial Sequence
  Cre R
gtgaaacagcattgctgtcactt

SEQ ID NO: 8 - DNA, 24 bp, Artificial Sequence
  Internal control F
ctaggccacagaattgaaagatct

SEQ ID NO: 9 - DNA, 25 bp, Artificial Sequence
  Internal Control R
gtaggtggaaattctagcatcatcc

SEQ ID NO: 10 - DNA, 20 bp, Artificial Sequence
  Human HECTD1 F
ccagtgagagtgacacaggt

SEQ ID NO: 11 - DNA, 20 bp, Artificial Sequence
  Human HECTD1 R
atcatcatcgagctcagcca

SEQ ID NO: 12 - DNA, 20 bp, Artificial Sequence
  Human ZNF622F
ggctcacatgaccaaagacc

SEQ ID NO: 13 - DNA, 20 bp, Artificial Sequence
  Human ZNF622 R
cgttgcaccacaagcaaatc

SEQ ID NO: 14 - DNA, 18 bp, Artificial Sequence
  Human GAPDH F
ccacccatggcaaattcc

SEQ ID NO: 15 - DNA, 22 bp, Artificial Sequence
  Human GAPDH R
tgggatttccattgatgacaag

SEQ ID NO: 16 - DNA, 20 bp, Artificial Sequence
  Mouse Hectd1-exon3 pair1 F
agcccgtgccataacatact

SEQ ID NO: 17 - DNA, 20 bp, Artificial Sequence
  Mouse Hectd1-exon3 pair1 R
tctctgctggtcctgttgtt

SEQ ID NO: 18 - DNA, 20 bp, Artificial Sequence
  Mouse Hectd1-exon3 pair2 F
actttcctgccagctctctg

SEQ ID NO: 19 - DNA, 20 bp, Artificial Sequence
  Mouse Hectd1-exon3 pair2 R
ctatcctccgggtgcattct

SEQ ID NO: 20 - DNA, 20 bp, Artificial Sequence
  Mouse Hectd1-exon6 F
ctggttgacctcctcttggt

SEQ ID NO: 21 - DNA, 20 bp, Artificial Sequence
  Mouse Hectd1-exon6 R
tctataagctgccgatggga

SEQ ID NO: 22 - DNA, 20 bp, Artificial Sequence
  Mouse Hectd1-exon22 F
gccgcttggaggacatacta

SEQ ID NO: 23 - DNA, 20 bp, Artificial Sequence
  Mouse Hectd1-exon22 R
agaccttccatagccacgag

SEQ ID NO: 24 - DNA, 20 bp, Artificial Sequence
  Mouse Hectd1-exon38 F
atgtacagcggtcatgtgga

SEQ ID NO: 25 - DNA, 20 bp, Artificial Sequence
  Mouse Hectd1-exon38 R
gtcgcccatgcacataagtt

SEQ ID NO: 26 - DNA, 20 bp, Artificial Sequence
  Mouse ZNF622 F
agaggaggagggagaggaag

SEQ ID NO: 27 - DNA, 20 bp, Artificial Sequence
  Mouse ZNF622 R
cactccagggtcctcacatt

SEQ ID NO: 28 - DNA, 21 bp, Artificial Sequence
  Mouse Rps6 F
agctccgcaccttctatgaga

SEQ ID NO: 29 - DNA, 22 bp, Artificial Sequence
  Mouse Rps6 R
gggaaaaccttgcttgtcattc

SEQ ID NO: 30 - DNA, 19 bp, Artificial Sequence
  Mouse Rps7 F
agcgccaagatcgtgaagc

SEQ ID NO: 31 - DNA, 22 bp, Artificial Sequence
  Mouse Rps7 R
caccaccaacttcgatttcctt

SEQ ID NO: 32 - DNA, 22 bp, Artificial Sequence
  Mouse Rpl5 F
ggaggtgaatggaggtgaatac

SEQ ID NO: 33 - DNA, 20 bp, Artificial Sequence
  Mouse Rpl5 R
agttgtagttcgggcaagac

SEQ ID NO: 34 - DNA, 19 bp, Artificial Sequence
  Mouse Rpl11 F
atggcgcaagatcaagggg

SEQ ID NO: 35 - DNA, 21 bp, Artificial Sequence
  Mouse Rpl11 R
gactgtgcagtgaacagcaat

SEQ ID NO: 36 - DNA, 1434 bp, Artificial Sequence
  Homo sapiens zinc finger protein 622
atggcgacgtacacctgcataacttgccgggtggcgttccgcgacgcggacatgcagcgggcccactataagacggactggcaccgctacaacctgcggcggaaggtggccagcatggccccagtgaccgccgagggcttccaggagcgagtgcgggcgcagcgggccgtcgcggaggaggagagcaagggctcggccacctactgcaccgtttgcagtaagaagtttgcctctttcaacgcctacgagaaccacctcaagtcccggcgtcacgttgagctggagaagaaggccgtgcaggcagtgaatcggaaagtggagatgatgaatgaaaagaacttggagaaaggactgggcgtggacagtgtggacaaggatgccatgaacgcggccatccagcaggccatcaaggcccagccgtccatgtctcccaagaaggcgcccccagcgcctgcaaaggaggccaggaatgtcgtggccgtgggtactggtggccgtgggacccacgaccgagacccgagtgagaaaccaccccggctccagtggtttgaacagcaggcgaagaagttggcaaagcagcaggaggaggacagcgaggaggaggaagaggacctggatggagacgattgggaagatattgattctgatgaagaattggaatgtgaggatactgaagcaatggacgatgtggtggagcaggatgcagaggaggaagaggctgaggaaggcccaccccttggtgccatccctatcacggactgcttattttgttcccatcattccagctcgctgatgaagaatgtggctcacatgaccaaagaccacagtttctttattcctgatatagaatatctttcagatattaagggactgattaaatacttgggagagaaagttggtgttggcaagatttgcttgtggtgcaacgagaaagggaagtccttctactccacagaagctgtacaggcacatatgaatgacaaaagccactgtaagctcttcacagatggcgatgctgctttggaatttgcagacttctatgattttaggagtagctatccagatcacaaggaaggggaggaccccaataaggctgaggagttgccctcagaaaagaacttggaatatgatgatgaaaccatggaattgattctgccttctggtgccagagtgggtcatcgctccttgatgagatactacaaacagcgatttggcttgtcaagagctgtggcagttgccaaaaatcggaaggccgtgggccgagtacttcagcagtacagagccctgggatggactggcagcacaggagcggctcttatgcgagagcgagacatgcagtatgtccaaaggatgaaatcaaaatggatgctgaagacaggaatgaagaacaatgccaccaagcagatgcactttcgggtccaagtgagattctga

SEQ ID NO: 37 - DNA, 19 bp, Artificial Sequence
  antisense1
tcatcatctccactttccg

SEQ ID NO: 38 - DNA, 19 bp, Artificial Sequence
  shRNA1
caaagaccacagtttcttt

SEQ ID NO: 39 - DNA, 22 bp, Artificial Sequence
  shRNA2
accaaagaccacagtttcttta

SEQ ID NO: 40 - DNA, 97 bp, Artificial Sequence
  zinc finger protein 622 start position 466
tgctgttgacagtgagcgcccaaagaccacagtttctttatagtgaagccacagatgtataaagaaactgtggtctttggttgcctactgcctcgga

SEQ ID NO: 41 - DNA, 19 bp, Artificial Sequence
  antisense2
aaagaaactgtggtctttg

SEQ ID NO: 42 - DNA, 19 bp, Artificial Sequence
  shRNA3
caaagaccacagtttcttt

SEQ ID NO: 43 - DNA, 22 bp, Artificial Sequence
  shRNA4
accaaagaccacagtttcttta

SEQ ID NO: 44 - DNA, 97 bp, Artificial Sequence; identical to SEQ ID NO: 40
  zinc finger protein 622 start position 952

SEQ ID NO: 45 - DNA, 23 bp, Artificial Sequence
  guide strand1
cacctgcataacttgccgggtgg

SEQ ID NO: 46 - DNA, 23 bp, Artificial Sequence
  guide strand2
ccgggtggcgttccgcgacgcgg

SEQ ID NO: 47 - DNA, 23 bp, Artificial Sequence
  guide strand3
gggcccactataagacggactgg

SEQ ID NO: 48 - DNA, 23 bp, Artificial Sequence
  guide strand4
tactgaagcaatggacgatgtgg

SEQ ID NO: 49 - DNA, 23 bp, Artificial Sequence
  guide strand5
cgtgatagggatggcaccaaggg

SEQ ID NO: 50 - DNA, 23 bp, Artificial Sequence
  guide strand6
taagcagtccgtgatagggatgg